(19) United States
(12) Patent Application Publication
(10) Pub. No.: US 2024/0092936 A1
(43) Pub. Date: Mar. 21, 2024

(54) METHODS OF TREATING HER2 POSITIVE BREAST CANCER WITH TUCATINIB IN COMBINATION WITH CAPECITABINE AND TRASTUZUMAB

(71) Applicant: Seagen Inc., Bothell, WA (US)

(72) Inventor: Luke Walker, Bothell, WA (US)

(21) Appl. No.: 17/768,290

(22) PCT Filed: Oct. 20, 2020
(86) PCT No.: PCT/US2020/056489

§ 371 Date: Apr. 12, 2022

Related U.S. Application Data
(60) Provisional application No. 62/923,659, filed on Oct. 21, 2019.

(51) Int. Cl. C07K 16/32; A61K 31/517; A61K 31/7068; A61K 45/06; A61P 35/04

(52) U.S. Cl. CPC C07K 16/32; A61K 31/517; A61K 31/7068; A61K 45/06; A61P 35/04; A61K 2039/505

(57) ABSTRACT

In one aspect, the present disclosure relates to a method for treating or ameliorating the effects of a HER2 positive breast cancer in a subject by administration of a combination of tucatinib, capecitabine, and trastuzumab. In some embodiments, the methods provided herein are useful for treating or ameliorating the effects of a HER2 positive breast cancer-associated brain metastasis in a subject by administration of a combination of tucatinib, capecitabine, and trastuzumab.

### CROSS-REFERENCE TO RELATED APPLICATION

This application claims the benefit of U.S. Provisional Application U.S. 62/923,659, filed on Oct. 21, 2019. The disclosure of the prior application is incorporated by reference in its entirety.

### BACKGROUND

Breast cancer is the most common form of cancer in women worldwide, and the second leading cause of cancer-related death in the United States. Approximately 20% of breast cancers overexpress the human epidermal growth factor receptor 2 (HER2). HER2 is a transmembrane tyrosine kinase receptor that mediates cell growth, differentiation, and survival. Tumors that overexpress HER2 are more aggressive and historically have been associated with poorer overall survival (OS) compared to HER2 negative cancers. Cancers that are characterized by the overexpression of HER2 (referred to as HER2 positive cancers) are often correlated with poor prognosis and/or are resistant to many standard therapies.

The introduction of HER2-targeted therapy using either antibody-based therapy or a small molecule tyrosine kinase inhibitor (TKI) has led to improvements in disease-free survival (DFS), progression-free survival (PFS), and OS in both the adjuvant and metastatic settings. Trastuzumab, a humanized anti-HER2 antibody, remains the backbone of treatment in the adjuvant and first-line metastatic settings, usually in combination with a taxane. Anti-HER2 therapy in combination with cytotoxic chemotherapy allows for concurrent treatment with agents having two different mechanisms of action, leading to greater efficacy than with either agent alone.

Despite the improvements in outcomes for early stage HER2+ breast cancer, up to a quarter of all patients treated with anti-HER2 therapy in the adjuvant setting relapse. The development of new HER2 targeted therapies such as pertuzumab and T-DM1 (ado-trastuzumab emtansine or trastuzumab emtansine) for metastatic HER2+ breast cancer has led to a meaningful prolongation in the median survival of these patients; however, essentially all patients in the metastatic setting ultimately progress. Treatment failures may result from primary or acquired resistance to HER2 blockade. There is evidence that dual targeting of HER2, either through combination of 2 different HER2-targeted antibodies or through use of an antibody-based therapy such as trastuzumab and a TKI, can lead to further improvements in efficacy in metastatic disease. In particular, combination of a small molecule TKI with an antibody-based therapy may be effective, as it may help overcome resistance to antibody-mediated inhibition through utilization of an alternative mechanism of receptor inhibition. Lapatinib, a dual epidermal growth factor receptor (EGFR)/HER2 oral TKI, has been shown to have increased activity in combination with trastuzumab compared to lapatinib alone, even when given to patients who have previously progressed on prior trastuzumab-based therapy. Use of lapatinib, however, has been limited by the anti-EGFR/human epidermal growth factor receptor 1 (HER1) activity of the drug, which results in toxicities such as rash, diarrhea, and fatigue.

The current standard of care for patients with HER2+ metastatic disease consists of treatment with pertuzumab plus trastuzumab and a taxane as first-line treatment for metastatic disease, followed by T-DM1 in second line. Treatment options for patients who progress after treatment with both pertuzumab and T-DM1 remain relatively limited. Patients are generally treated with a continuation of anti-HER2 therapy (in the form of trastuzumab or lapatinib) in combination with cytotoxic chemotherapy, such as capecitabine. Combined HER2 therapy with trastuzumab and lapatinib can also be considered. However, no single regimen is considered the standard of care in this setting and better options for these patients are needed.

The treatment and prevention of brain metastases represents an unmet medical need in the post-trastuzumab era. Recent data suggest that the incidence of first relapse occurring in the brain is increasing in patients who have received trastuzumab-based adjuvant therapy, and approximately 30-50% of HER2+ patients with metastatic disease will develop brain metastases. The increasing prevalence of brain metastases in HER2+ breast cancer patients may be due to several factors. First, HER2+ breast cancer appears to display tropism for the brain. Second, with better control of non-CNS disease, patients may be living longer allowing brain metastases to become more of a critical clinical issue. Finally, the brain may represent a sanctuary site for HER2+ disease as large molecules, such as trastuzumab, do not penetrate the blood-brain barrier. Treatment options for brain metastases are limited. There is no specific systemic treatment regimen approved for brain metastases, and treatment currently relies heavily on the use of local therapies such as whole brain radiation therapy (WBRT), stereotactic radiation (SRS), or surgery. Patients may also receive chemotherapy alone, or capecitabine and either lapatinib or trastuzumab, although brain response rates are generally modest. The development of HER2-targeted systemic therapies with clinical benefit in both brain and non-CNS sites of disease could lead to improved clinical outcomes, both by improving overall PFS and OS as well as by avoiding or delaying the use of radiation therapy and its associated toxicities, including neurocognitive impairment.

Accordingly, there is a need for new therapies that are effective for the treatment of patients with HER2-positive breast cancer (e.g., patients with unresectable, locally advanced, or metastatic HER2-positive breast cancer, including patients with brain metastases).

All references cited herein, including patent applications, patent publications, and scientific literature, are herein incorporated by reference in their entirety, as if each individual reference were specifically and individually indicated to be incorporated by reference.

### SUMMARY

Provided herein are methods for treating or ameliorating a HER2 positive breast cancer in a subject in need thereof, the methods comprising administering to the subject an effective amount of a combination therapy comprising tucatinib, capecitabine, and trastuzumab, wherein following administration of the combination therapy, the subject exhibits progression-free survival of at least 7.5 months following administration of the combination therapy. For example, wherein the subject exhibits progression-free survival of at least eight months, at least nine months, or at least ten months following administration of the combination therapy.

Also provided herein are methods for treating or ameliorating a HER2 positive breast cancer in a subject in need thereof, the methods comprising administering to the subject an effective amount of a combination therapy comprising tucatinib, capecitabine, and trastuzumab, wherein following administration of the combination therapy, the subject exhibits an overall survival of at least eighteen months following administration of the combination therapy. For example, wherein the subject exhibits an overall survival of at least nineteen months, at least twenty-two months, at least twenty-six months, or at least thirty months following administration of the combination therapy.

In some of the embodiments as provided herein, the subject has a brain metastasis.

Accordingly, provided herein are methods of treating or ameliorating brain metastasis in a subject having HER2 positive breast cancer, the methods comprising administering to the subject an effective amount of a combination therapy comprising tucatinib, capecitabine, and trastuzumab. In some embodiments, the time to additional intervention (e.g., radiation, surgery, or a combination thereof) for treatment of the brain metastasis in the subject has been increased. In some embodiments, the need for additional intervention (e.g., radiation, surgery, or a combination thereof) for treatment of the brain metastasis in the subject has been prevented. In some embodiments, regression of an existing brain metastasis in the subject has been promoted. In some embodiments, the size of an existing brain metastasis in the subject has been reduced.

This disclosure also provides methods for treating or ameliorating a HER2 positive breast cancer in a subject in need thereof, wherein the subject has brain metastasis, the methods comprising administering to the subject an effective amount of a combination therapy comprising tucatinib, capecitabine, and trastuzumab, wherein following administration of the combination therapy, the subject exhibits progression-free survival of at least 6 months following administration of the combination therapy. For example, the subject can exhibit progression-free survival of at least seven months or at least nine months following administration of the combination therapy.

Methods for treating or ameliorating a HER2 positive breast cancer in a subject in need thereof are also provided, the methods comprising administering to the subject an effective amount of a combination therapy comprising tucatinib, capecitabine, and trastuzumab, wherein the subject exhibits a greater than 40% reduction in the risk of disease progression or death as compared to a subject administered trastuzumab and capecitabine alone. For example, the subject can exhibit a greater than 45% reduction in the risk of disease progression or death as compared to a subject administered trastuzumab and capecitabine alone.

Provided herein are methods for treating or ameliorating a HER2 positive breast cancer in a subject in need thereof, the methods comprising administering to the subject an effective amount of a combination therapy comprising tucatinib, capecitabine, and trastuzumab, wherein the subject exhibits a greater than 30% reduction in the risk of death as compared to a subject administered trastuzumab and capecitabine alone.

Also provided herein are methods for treating or ameliorating a HER2 positive breast cancer in a subject in need thereof, wherein the subject has a brain metastasis, the methods comprising administering to the subject an effective amount of a combination therapy comprising tucatinib, capecitabine, and trastuzumab, wherein the subject exhibits a greater than 50% reduction in the risk of disease progression or death as compared to a subject administered trastuzumab and capecitabine alone.

Further provided herein are methods for treating or ameliorating a HER2 positive breast cancer in a subject in need thereof, the methods comprising administering to the subject an effective amount of a combination therapy comprising tucatinib, capecitabine, and trastuzumab, wherein following administration of the combination therapy for nine months, the subject has an estimated progression-free survival rate of greater than 40%. For example, wherein the subject has an estimated progression-free survival rate of greater than 45% following administration of the combination therapy for nine months.

This disclosure also provides methods for treating or ameliorating a HER2 positive breast cancer in a subject in need thereof, the methods comprising administering to the subject an effective amount of a combination therapy comprising tucatinib, capecitabine, and trastuzumab, wherein following administration of the combination therapy for twelve months, the subject has an estimated progression-free survival rate of greater than 25%. For example, wherein the subject has an estimated progression-free survival rate of greater than 30% following administration of the combination therapy for twelve months.

Methods for treating or ameliorating a HER2 positive breast cancer in a subject in need thereof are provided herein, the methods comprising administering to the subject an effective amount of a combination therapy comprising tucatinib, capecitabine, and trastuzumab, wherein following administration of the combination therapy for fifteen months, the subject has an estimated progression-free survival rate of greater than 20%. For example, wherein the subject has an estimated progression-free survival rate of greater than 25% following administration of the combination therapy for fifteen months.

Provided herein are methods for treating or ameliorating a HER2 positive breast cancer in a subject in need thereof, the methods comprising administering to the subject an effective amount of a combination therapy comprising tucatinib, capecitabine, and trastuzumab, wherein following administration of the combination therapy for twenty-four months, the subject has an estimated overall survival rate of greater than 35%. For example, wherein the subject has an estimated overall survival rate of greater than 40% following administration of the combination therapy for twenty-four months.

Also provided herein are methods for treating or ameliorating a HER2 positive breast cancer in a subject in need thereof, the methods comprising administering to the subject an effective amount of a combination therapy comprising tucatinib, capecitabine, and trastuzumab, wherein following administration of the combination therapy for thirty months, the subject has an estimated overall survival rate of greater than 30%. For example, wherein the subject has an estimated overall survival rate of greater than 40% following administration of the combination therapy for thirty months.

Further provided herein are methods for treating or ameliorating a HER2 positive breast cancer in a subject in need thereof, wherein the subject has a brain metastasis, the methods comprising administering to the subject an effective amount of a combination therapy comprising tucatinib, capecitabine, and trastuzumab, wherein following administration of the combination therapy for nine months, the subject has an estimated progression-free survival rate of greater than 30%. For example, wherein the subject has an estimated progression-free survival rate of greater than 40% following administration of the combination therapy for nine months.

This disclosure also provides methods for treating or ameliorating a HER2 positive breast cancer in a subject in need thereof, wherein the subject has a brain metastasis, the methods comprising administering to the subject an effective amount of a combination therapy comprising tucatinib, capecitabine, and trastuzumab, wherein following administration of the combination therapy for twelve months, the subject has an estimated progression-free survival rate of greater than 15%. For example, wherein following administration of the combination therapy for twelve months, the subject has an estimated progression-free survival rate of greater than 20%.

Methods for treating or ameliorating a HER2 positive breast cancer in a subject in need thereof are provided herein, the methods comprising administering to the subject an effective amount of a combination therapy comprising tucatinib, capecitabine, and trastuzumab and an effective amount of an anti-diarrheal agent. Also provided herein are method of reducing the severity or incidents of diarrhea, or preventing diarrhea in a subject having a HER2 positive breast cancer and being treated with an effective amount of a combination therapy comprising tucatinib, capecitabine, and trastuzumab, the method comprising administering an effective amount of an anti-diarrheal agent prophylactically. This disclosure also provides methods of reducing the likelihood of a subject developing diarrhea, wherein the subject has a HER2 positive breast cancer and is being treated with an effective amount of a combination therapy comprising tucatinib, capecitabine, and trastuzumab, the method comprising administering an effective amount of an anti-diarrheal agent prophylactically. In some such methods, the combination therapy and the anti-diarrheal agent can be administered concurrently. In some such methods, the anti-diarrheal agent is administered prior to administration of the combination therapy. In some embodiments, the subject is exhibiting symptoms of diarrhea. In other embodiments, the subject is not exhibiting symptoms of diarrhea.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the disclosure described herein include “comprising,” “consisting,” and “consisting essentially of” aspects and embodiments. It is understood that aspects and variations of the embodiments described herein include “consisting of” and/or “consisting essentially of” aspects and variations. In some embodiments, methods consisting essentially of an administration step as disclosed herein include methods wherein a patient has failed a prior therapy (administered to the patient before the period of time) or has been refractory to such prior therapy, and/or wherein the cancer has metastasized or recurred. In some embodiments, methods consisting essentially of an administration step as disclosed herein include methods wherein a patient undergoes surgery, radiation, and/or other regimens prior to, substantially at the same time as, or following such an administration step as disclosed herein, and/or where the patient is administered other chemical and/or biological therapeutic agents following such an administration step as disclosed herein.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “comprising” as used herein should in general be construed as not excluding additional ingredients. For example, a claim to “a composition comprising A” would cover compositions that include A and B; A, B, and C; A, B, C, and D; A, B, C, D, and E; and the like.

As used herein, the term “co-administering” includes sequential or simultaneous administration of tucatinib, capecitabine, and trastuzumab. For example, the co-administered compounds are administered by the same route. In other instances, the co-administered compounds are administered via different routes. For example, one or two compounds can be administered orally, and the other compound(s) can be administered, e.g., sequentially or simultaneously, via intravenous, intramuscular, subcutaneous, or intraperitoneal injection. The simultaneously or sequentially administered compounds or compositions can be administered such that trastuzumab, capecitabine, and tucatinib are simultaneously present in a subject or in a cell at an effective concentration.

A “cancer” refers to a broad group of various diseases characterized by the uncontrolled growth of abnormal cells in the body. A “cancer” or “cancer tissue” can include a tumor.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “TO” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP_001005862, NP_001276865, NP_001276866, NP_001276867, and NP_004439.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression.

The term “HER2 positive-associated” with respect to a disease or disorder, as used herein refers to diseases or disorders associated with amplification or overexpression of HER2. Non-limiting examples of HER2 positive-associated diseases or disorders include, for example, HER2 positive breast cancer (e.g., “HER2 positive breast cancer-associated”).

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Following metastasis, the distal tumors can be said to be “derived from” the pre-metastasis tumor. For example, a “tumor derived from” a breast cancer refers to a tumor that is the result of a metastasized breast cancer. Metastatic brain cancer refers to cancer in the brain, i.e., cancer which originated in a tissue other than the brain and has metastasized to the brain.

The term “tucatinib,” also known as ONT-380 and ARRY-380, refers to the small molecule tyrosine kinase inhibitor that suppresses or blocks HER2 activation. Tucatinib has the following structure:

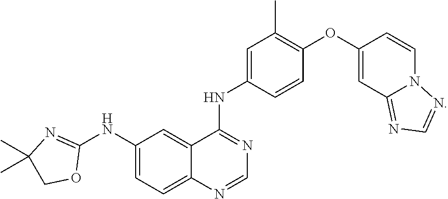 [SMILES: Cc1cc(Nc2ncnc3ccc(NC4=NC(C)(C)CO4)cc23)ccc1Oc1ccn2ncnc2c1]

In some instances, tucatinib can be in the form of a pharmaceutically acceptable salt.

The term “anti-HER2 antibody” refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present disclosure include trastuzumab, pertuzumab, ado-trastuzumab emtansine (also known as T-DM1), margetuximab, and combinations or biosimilars thereof.

The term “trastuzumab” refers to an anti-HER2 monoclonal antibody used to treat breast cancer and sold under the tradenames Herceptin, Ogivri, and Herzuma. As used herein, “trastuzumab” also includes biosimilars, for example, Kanjinti (trastuzumab-anns).

A “biosimilar” as used herein refers to an antibody or antigen-binding fragment that has the same primary amino acid sequence as compared to a reference antibody (e.g., trastuzumab) and optionally, may have detectable differences in post-translation modifications (e.g., glycosylation and/or phosphorylation) as compared to the reference antibody (e.g., a different glycoform).

In some embodiments, a biosimilar is an antibody or antigen-binding fragment thereof that has a light chain that has the same primary amino acid sequence as compared to a reference antibody (e.g., trastuzumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., trastuzumab) and a heavy chain that includes the same heavy chain variable domain sequence as a reference antibody. In some embodiments, a biosimilar can have a similar glycosylation pattern as compared to the reference antibody (e.g., trastuzumab). In other embodiments, a biosimilar can have a different glycosylation pattern as compared to the reference antibody (e.g., trastuzumab).

The term “capecitabine” refers to a prodrug of fluorouracil having the following structure:

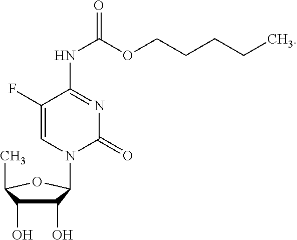

Capecitabine undergoes hydrolysis in the liver and tissues to form fluorouracil which is the active moiety. Fluorouracil is a fluorinated pyrimidine antimetabolite that inhibits thymidylate synthetase, blocking the methylation of deoxyuridylic acid to thymidylic acid, interfering with DNA, and to a lesser degree, RNA synthesis.

The term “tumor growth inhibition (TGI) index” refers to a value used to represent the degree to which an agent (e.g., tucatinib, capecitabine, trastuzumab, or a combination thereof) inhibits the growth of a tumor when compared to an untreated control. The TGI index is calculated for a particular time point (e.g., a specific number of days into an experiment or clinical trial) according to the following formula:

$$T G I = 1 - ( \frac{{Volume}_{t r e a t e d ( Tx Day X )} - {Volume}_{t r e a t e d ( Tx Day 0 )}}{{Volume}_{c o n t r o l ( Tx Day X )} - {Volume}_{c o n t r o l ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib, capecitabine, and trastuzumab) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and asumes that the drugs act completely independently (i.e., the drugs do not interfere with one another (e.g., the drugs have different sites of action) but each contributes to a common result).

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, a combination of tucatinib, capecitabine, and trastuzumab is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression (e.g., brain metastasis regression) can be observed and continue for a period of at least about 20 days, at least about 30 days, at least about 40 days, at least about 50 days, or at least about 60 days.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

“Simultaneous administration,” as used herein, means that the two or more therapies (e.g., in a combination therapy) are administered with a time separation of no more than about 15 minutes, such as no more than about any of 10, 5, or 1 minutes. When the two or more therapies are administered simultaneously, the two or more therapies can be contained in the same composition (e.g., a composition comprising both a first and second therapy) or in separate compositions (e.g., a first therapy in one composition and a second therapy is contained in another composition).

As used herein, the term “sequential administration” means that the two or more therapies (e.g., in a combination therapy) are administered with a time separation of more than about 15 minutes, such as more than about any of 20, 30, 40, 50, 60, or more minutes. Any of the two or more therapies may be administered first. The two or more therapies are contained in separate compositions, which may be contained in the same or different packages or kits.

As used herein, the term “concurrent administration” means that the administration of two or more therapies (e.g., in a combination therapy) overlap with each other. For example, the two or more therapies may be administered in the same day, or with a time separation of within one day, within two days, within three days, within four days, within five days, within six days, within seven days, within ten days, within fourteen days, or within twenty-one days.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., breast cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 6.0 mg/kg of an agent, such as trasuzumab, one can calculate and use the appropriate amount of the agent (i.e., 360 mg) for administration to said subject.

The use of the term “fixed dose” with regard to a method of the disclosure means that two or more different agents (e.g., two or more of tucatinb, capecitabine, and trastuzumab) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents. For example, a 1:2 ratio of tucatinib to trastuzumab administered to a subject can mean about 300 mg of tucatinib and about 600 mg of trastuzumab or about 3 mg/ml of tucatinib and about 6 mg/ml of trastuzumab are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib, capecitabine, or trastuzumab). For example, a subject with 60 kg body weight and a subject with 100 kg body weight would receive the same dose of tucatinb (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis 42-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion., a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

As used herein, the term “solid dispersion” means a system in a solid state comprising at least two components, wherein one component is dispersed throughout the other component. For example, a solid dispersion as described herein can include one component of tucatinib dispersed throughout another component, such as a dispersion polymer.

As used herein, the term “amorphous” means a solid in a solid state that is a non-crystalline state. Amorphous solids generally possess crystal-like short range molecular arrangement, but no long range order of molecular packing as found in crystalline solids. The solid state form of a solid may be determined by polarized light microscopy, X-ray powder diffraction (“XRPD”), differential scanning calorimetry (“DSC”), or other standard techniques known to those of skill in the art.

As used herein, the term “amorphous solid dispersion” means a solid comprising a drug substance and a dispersion polymer. The amorphous solid dispersion discussed herein comprises amorphous tucatinib and a dispersion polymer, wherein the amorphous solid dispersion contains tucatinib in a substantially amorphous solid state form. In certain embodiments, the substantially amorphous solid state form means that the tucatinib component in the amorphous solid dispersion is at least 80% amorphous tucatinib. In certain embodiments, the substantially amorphous solid state form means that the tucatinib component in the amorphous solid dispersion is at least 85% amorphous tucatinib. In certain embodiments, the substantially amorphous solid state form means that the tucatinib component in the amorphous solid dispersion is at least 90% tucatinib. In certain embodiments, the substantially amorphous solid state form means that the tucatinib component in the amorphous solid dispersion is at least 95% amorphous tucatinib.

As used herein, the term “dispersion polymer” means a polymer that allows for tucatinib to be dispersed throughout such that a solid dispersion may form. The dispersion polymer is preferably neutral or basic. The dispersion polymer may contain a mixture of two or more polymers. Examples of dispersion polymers include, but are not limited to, vinyl polymers and copolymers, vinylpyrrolidine vinylacetate copolymer (“PVP-VA”), polyvinyl alcohols, polyvinyl alcohol polyvinyl acetate copolymers, polyvinyl pyrrolidine (“PVP”), acrylate and methacrylate copolymers, methylacrylic acid methyl methacrylate copolymer (such as Eudragit®), polyethylene polyvinyl alcohol copolymers, polyoxyethylene-polyoxypropylene block copolymers (also referred to as poloxamers), graft copolymer comprised of polyethylene glycol, polyvinyl caprolactam and polyvinyl acetate (such as Soluplus®), cellulosic polymers, such as hydroxypropyl methyl cellulose acetate (“HPMCA”), hydroxypropyl methyl cellulose (“HPMC”), hydroxypropyl cellulose (“HPC”), methyl cellulose, hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate (“HPMCAT”), and carboxymethylcellulose acetate butyrate (“CMCAB”), and the like.

As used herein, the term “spray drying” means processes involved in breaking up liquid mixtures into small droplets (atomization) and rapidly removing solvent from the mixture in a spray drying apparatus where there is a strong driving force for evaporation of solvent from the droplets. The phrase spray drying is used conventionally and broadly. Spray drying processes and spray drying equipment are described generally in Perry, Robert H., and Don W. Green (eds.). Perry's Chemical Engineers' Handbook. New York: McGraw-Hill, 2007 (8^{th} edition).

As used herein, “polymorphs” refer to distinct solids sharing the same molecular formula, yet each polymorph may have distinct solid state physical properties. A single compound may give rise to a variety of polymorphic forms where each form has different and distinct solid state physical properties, such as different solubility profiles, melting point temperatures, flowability, dissolution rates and/or different X-ray diffraction peaks. These practical physical characteristics are influenced by the conformation and orientation of molecules in the unit cell, which defines a particular polymorphic form of a substance. Polymorphic forms of a compound can be distinguished in a laboratory by X-ray diffraction spectroscopy, such as X-ray powder diffraction (“XRPD”), and by other methods, such as infrared spectrometry. Additionally, polymorphic forms of the same drug substance or active pharmaceutical ingredient can be administered by itself or formulated as a drug product (pharmaceutical composition) and are well known in the pharmaceutical art to affect, for example, the solubility, stability, flowability, tractability and compressibility of drug substances and the safety and efficacy of drug products. For more, see Hilfiker, Rolf (ed.), Polymorphism in the Pharmaceutical Industry. Weinheim, Germany: Wiley-VCH 2006.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., breast cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±two days, i.e., every twelve days to every sixteen days. “Once about every three weeks” can include every twenty-one days ±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating Breast Cancer with Tucatinib in Combination with Capecitabine and Trastuzumab

The 2014 World Cancer Report from WHO (The World health organization) reports that breast cancer is the second most common cancer worldwide, accounting for just over I million new cases annually. It states that in 2000 about 400,000 women died from breast cancer, representing 1.6 percent of all female deaths. The proportion of breast cancer deaths was far higher in the rich countries (2 percent of all female deaths) than in economically poor regions (0.5 percent). Thus, breast cancer is strongly related to the Western lifestyle. As developing countries succeed in achieving lifestyles similar to Europe, North America, Australia, New Zealand and Japan, they will also encounter much higher cancer rates, particularly cancers of the breast. Recent data supports this prediction and show a 20% increase in breast cancer from 2008 to 2012. (Carter D. “New global survey shows an increasing cancer burden”. Am J Nurs. 2014 March; 114(3): 17).

In some aspects, the disclosure provides a method for treating breast cancer in a subject comprising administering a combination of tucatinib, capecitabine, and trastuzumab as described herein. In some embodiments, the breast cancer is a HER2 positive breast cancer. In some embodiments, the cancer is determined to be HER2 positive using in situ hybridization, fluorescence in situ hybridization, or immunohistochemistry. In some embodiments, the breast cancer is metastatic. In some embodiments, the breast cancer has metastasized to the brain. In some embodiments, the breast cancer is locally advanced. In some embodiments, the breast cancer is unresectable.

In some embodiments, the subject has been previously treated with one or more additional therapeutic agents for the breast cancer. In some embodiments, the subject has been previously treated with one or more additional therapeutic agents for the breast cancer and did not respond to the treatment. In some embodiments, the subject has been previously treated with one or more additional therapeutic agents for the breast cancer and relapsed after the treatment. In some embodiments, the subject has been previously treated with one or more additional therapeutic agents for the breast cancer and experienced disease progression during the treatment. In some embodiments, the one or more additional therapeutic agents is an anti-HER2 antibody or anti-HER2 antibody-drug conjugate. In some embodiments, the one or more additional therapeutic agents is an anti-HER2 antibody. In some embodiments, the one or more additional therapeutic agents is anti-HER2 antibody-drug conjugate. In some embodiments, the subject has been previously treated with trastuzumab, pertuzumab and/or T-DM1. In some embodiments, the subject has been previously treated with trastuzumab. In some embodiments, the subject has been previously treated with pertuzumab. In some embodiments, the subject has been previously treated with T-DM1. In some embodiments, the subject has been previously treated with trastuzumab and pertuzumab. In some embodiments, the subject has been previously treated with trastuzumab and T-DM1. In some embodiments, the subject has been previously treated with pertuzumab and T-DM1. In some embodiments, the subject has been previously treated with trastuzumab, pertuzumab and T-DM1.

In some embodiments, the one or more additional therapeutic agents are selected from the group consisting of chemotherapeutic agents such as doxorubicin and cyclophosphamide (e.g., ACTH regimen); taxane (e.g., paclitaxel); docetaxel; docetaxel and carboplatin (e.g., TCH regimen); cisplatin; fluorouracil (5-FU); epirubicin; anthracyclines (e.g., doxorubicin); cyclophosphamide; capecitabine; vinorelbine; gemcitabine; kinase inhibitors, such as lapatinib; neratinib; pyrotinib; afatinib; poziotinib; abemaciclib; and pazopanib; anti-HER2 antibodies and/or antibody-drug conjugates such as trastuzumab; pertuzumab; MGAH22; MCLA-128; ZW25; GBR1302; PRS-343; T-DM1 or ado-trastuzumab emtansine; trastuzumab deruxtecan; SYD985; XMT-1522; ARX788; DHES0815A; MEDI4276; ADCT-502; and ertumaxomab; hormone therapy, including, for example tamoxifen; toremifene; fulvestrant; aromatase inhibitors (e.g., anastrozole, exemestane, letrozole); and ovarian suppression (e.g., with goserelin or leuprolide); vaccines such as nelipepimut-S or E75 peptide combined with granulocyte macrophage-colony stimulating factor; and ETBX-021; combination therapies such as a chemotherapeutic agent and trastuzumab (and optionally pertuzumab); taxane (e.g., paclitaxel) with trastuzumab; taxane (e.g., paclitaxel) with trastuzumab and pertuzumab; cis-platin and fluoropyrimidine with trastuzumab; docetaxel and carboplatin with trastuzumab and pertuzumab; docetaxel and carboplatin with trastuzumab; docetaxel with trastuzumab and pertuzumab; docetaxel with trastuzumab; docetaxel and cyclophosphamide with trastuzumab; anthracycline and/or cyclophosphamide followed by paclitaxel with trastuzumab; pertuzumab with docetaxel; fluorouracil (5-FU), epirubicin, and cyclophosphamide with trastuzumab and/or pertuzumab; vinorelbine or gemcitabine with trastuzumab; anthracycline, a taxane, and trastuzumab; doxorubicin with trastuzumab; lapatinib with capecitabine; lapatinib with trastuzumab; endocrine therapy with lapatinib and/or trastuzumab; pazopanib with lapatinib; anti-HER2 agents (e.g., trastuzumab) with CDK4/6 inhibitors (e.g., abemaciclib or palbociclib) such as abemaciclib with trastuzumab; palbociclib with trastuzumab, pertuzumab, and an aromatase inhibitor; palbociclib, trastuzumab (and optionally letrozole); palbociclib and T-DM1; palbociclib with trastuzumab, pertuzumab and anastrozole; ribociclib with trastuzumab or T-DM1; palbociclib with tucatinib and letrozole; anti-HER2 agents (e.g., trastuzumab, pertuzumab, T-DM1) with immunotherapy (e.g., with pembrolizumab, atezolizumab or nivolumab); anti-HER2 agents (e.g., trastuzumab, pertuzumab, T-DM1) with PI3K/AKT/mTOR inhibitors, for example, everolimus with trastuzumab and paclitaxel; everolimus with trastuzumab and vinorelbine; alpelisib with LJM716 and trastuzumab; alpelisib and T-DM1; taselisib with anti-HER2 agents (e.g., trastuzumab, trastuzumab emtansine, pertuzumab (and optionally paclitaxel)); and copanlisib with trastuzumab;

In some embodiments, the subject has been previously treated with one or more additional therapies for the breast cancer. For example, radiation (e.g., external beam radiation; brachytherapy), surgery (e.g., lumpectomy; mastectomy), and combinations thereof.

In some embodiments, the subject is refractory to the previous treatment. In some embodiments, the subject developed one or more brain metastasis while on the previous treatment.

In some embodiments, the subject has not been previously treated with another therapeutic agent for the breast cancer within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not been previously treated with another therapeutic agent for the breast cancer within the past 12 months prior to being administered the therapeutically effective amount of tucatinib, or salt or solvate thereof. In some embodiments, the subject has not been previously treated with another therapeutic agent for the breast cancer. In some embodiments, the subject has not been previously treated with lapatinib, neratinib, afatinib, or capecitabine. In some embodiments, the subject has not been previously treated with lapatinib. In some embodiments, the subject has not been previously treated with neratinib. In some embodiments, the subject has not been previously treated with afatinib. In some embodiments, the subject has not been previously treated with capecitabine.

In some embodiments, ther HER2 status of a sample cell is determined. The determination can be made before treatment (i.e., administration of a combination of tucatinib, capecitabine, and trastuzumab) begins, during treatment, or after treatment has been completed. In some instances, determination of the HER2 status results in a decision to change therapy (e.g., adding an anti-HER2 antibody to the treatment regimen, discontinuing the use of the combination of tucatinib, capecitabine, and trastuzumab, discontinuing therapy altogether, or switching from another treatment method to a method of the present disclosure).

In some embodiments, the sample cell is determined to be overexpressing or not overexpressing HER2. In particular embodiments, the cell is determined to be HER2 3+, HER2 2+, HER2 1+, or HER2 0 (i.e., HER is not overexpressed).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2. In other embodiments, the sample cell is determined to be HER2 positive when the HER2 gene copy number is greater than 6.

In one embodiment of the methods or uses or product for uses described herein, response to treatment with a combination of tucatinib, capecitabine, and trastuzumab described herein is assessed by measuring the time of progression free survival after administration of the combination of tucatinib, capecitabine, and trastuzumab. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the combination of tucatinib, capecitabine, and trastuzumab. In some embodiments, the subject exhibits progression-free survival of at least about 6 months after administration of the combination of tucatinib, capecitabine, and trastuzumab. In some embodiments, the subject exhibits progression-free survival of at least about one year after administration of the combination of tucatinib, capecitabine, and trastuzumab. In some embodiments, the subject exhibits progression-free survival of at least about two years after administration of the combination of tucatinib, capecitabine, and trastuzumab. In some embodiments, the subject exhibits progression-free survival of at least about three years after administration of the combination of tucatinib, capecitabine, and trastuzumab. In some embodiments, the subject exhibits progression-free survival of at least about four years after administration of the combination of tucatinib, capecitabine, and trastuzumab. In some embodiments, the subject exhibits progression-free survival of at least about five years after administration of the combination of tucatinib, capecitabine, and trastuzumab. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the combination of tucatinib, capecitabine, and trastuzumab. In some embodiments, the subject exhibits progression-free survival of at least 6 months after administration of the combination of tucatinib, capecitabine, and trastuzumab. In some embodiments, the subject exhibits progression-free survival of at least one year after administration of the combination of tucatinib, capecitabine, and trastuzumab. In some embodiments, the subject exhibits progression-free survival of at least two years after administration of the combination of tucatinib, capecitabine, and trastuzumab. In some embodiments, the subject exhibits progression-free survival of at least three years after administration of the combination of tucatinib, capecitabine, and trastuzumab. In some embodiments, the subject exhibits progression-free survival of at least four years after administration of the combination of tucatinib, capecitabine, and trastuzumab. In some embodiments, the subject exhibits progression-free survival of at least five years after administration of the combination of tucatinib, capecitabine, and trastuzumab.

In one aspect, provided herein are methods for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy, the subject exhibits progression-free survival of at least 7.5 months following administration of the combination therapy. For example, the subject can exhibit progression-free survival of at least eight months, of at least nine months, or at least ten months following administration of the combination therapy. In some embodiments, a subject can exhibit progression-free survival of 7.5 months, 7.6 months, 7.7 months, 7.8 months, 7.9 months, 8 months, 8.2 months, 8.5 months, 8.8 months, 9.6 months, 9.8 months, and 10 months following administration of the combination therapy.

Also provided herein is a method for treating or ameliorating a HER2 positive breast cancer in a subject in need thereof, the method comprising administering to the subject an effective amount of a combination therapy comprising tucatinib, capecitabine, and trastuzumab, wherein the subject exhibits a greater than 40% reduction in the risk of disease progression or death as compared to a subject administered trastuzumab and capecitabine alone. For example, the subject administered the combination therapy comprising tucatinib, capecitabine, and trastuzumab exhibits a greater than 45% reduction in the risk of disease progression or death as compared to a subject administered trastuzumab and capecitabine alone. In some embodiments, the subject exhibits a 46% reduction in the risk of disease progression or death.

In some embodiments as described herein, following administration of the combination therapy comprising tucatinib, capecitabine, and trastuzumab for nine months, the subject has an estimated progression-free survival rate of greater than 40%. For example, the subject has an estimated progression-free survival of 40.5%, 41%, 42%, 43%, 43.6%, 44%, 44.4%, 45%, 45.8%, 46%, 46.8%, 47%, 47.9%, 48%, 48.2%, 48.8%, 49%, 49.7%, 50%, 50.5%, 51%, 52,4%, 52%, 52.9%, 53%, 54%, or 55%. In some embodiments, the subject has an estimated progression-free survival rate of greater than 45%, greater than 50%, or greater than 55% following administration of combination therapy comprising tucatinib, capecitabine, and trastuzumab for nine months.

In some embodiments as described herein, following administration of the combination therapy comprising tucatinib, capecitabine, and trastuzumab twelve months, the subject has an estimated progression-free survival rate of greater than 25%. For example, the subject has an estimated progression-free survival of 25.4%, 26%, 26.6%, 27%, 27.4%, 28%, 28.6%, 29%, 29.3%, 30%, 30.7%, 31%, 31.5%, 32%, 32.8%, 33%, 33.1%, 34%, 34.4%, 35%, 35.5%, 36%, 36.8%, 37%, 37.3%, 38%, 38.6%, 39.7%, or 40%. In some embodiments, the subject has an estimated progression-free survival rate of greater than 30%, greater than 33%, greater than 35% following administration of combination therapy comprising tucatinib, capecitabine, and trastuzumab for twelve months.

In some embodiments as described herein, following administration of the combination therapy comprising tucatinib, capecitabine, and trastuzumab fifteen months, the subject has an estimated progression-free survival rate of greater than 20%. For example, the subject has an estimated progression-free survival of 20.2%, 20.5%, 21%, 21.3%, 22%, 22.6%, 23%, 23.7%, 24%, 24.4%, 25%, 25.6%, 26%, 26.2%, 27%, 27.4%, 28%, 28.6%, 29%, 29.3%, 30%, 30.7%, 31%, 31.5%, 32%, 32.8%, 33%, 33.8%, or 34%, In some embodiments, the subject has an estimated progression-free survival rate of greater than 25%, greater than 27%, greater than 30%, or greater than 33% following administration of the combination therapy comprising tucatinib, capecitabine, and trastuzumab for fifteen months.

In one embodiment of the methods or uses or product for uses described herein, response to treatment with a combination of tucatinib, capecitabine, and trastuzumab as described herein is assessed by measuring the time of overall survival after administration of the combination of tucatinib, capecitabine, and trastuzumab. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the combination of tucatinib, capecitabine, and trastuzumab. In some embodiments, the subject exhibits overall survival of at least about 6 months after administration of the combination of tucatinib, capecitabine, and trastuzumab. In some embodiments, the subject exhibits overall survival of at least about one year after administration of the combination of tucatinib, capecitabine, and trastuzumab. In some embodiments, the subject exhibits overall survival of at least about two years after administration of the combination of tucatinib, capecitabine, and trastuzumab. In some embodiments, the subject exhibits overall survival of at least about three years after administration of the combination of tucatinib, capecitabine, and trastuzumab. In some embodiments, the subject exhibits overall survival of at least about four years after administration of the combination of tucatinib, capecitabine, and trastuzumab. In some embodiments, the subject exhibits overall survival of at least about five years after administration of the combination of tucatinib, capecitabine, and trastuzumab. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the combination of tucatinib, capecitabine, and trastuzumab. In some embodiments, the subject exhibits overall survival of at least 6 months after administration of the combination of tucatinib, capecitabine, and trastuzumab. In some embodiments, the subject exhibits overall survival of at least one year after administration of the combination of tucatinib, capecitabine, and trastuzumab. In some embodiments, the subject exhibits overall survival of at least two years after administration of the combination of tucatinib, capecitabine, and trastuzumab. In some embodiments, the subject exhibits overall survival of at least three years after administration of the combination of tucatinib, capecitabine, and trastuzumab. In some embodiments, the subject exhibits overall survival of at least four years after administration of the combination of tucatinib, capecitabine, and trastuzumab. In some embodiments, the subject exhibits overall survival of at least five years after administration of the combination of tucatinib, capecitabine, and trastuzumab.

In one aspect, the present disclosure provides methods for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy, the subject exhibits an overall survival of at least eighteen months following administration of the combination therapy. For example, the subject can exhibit an overall survival of at least nineteen months following administration of the combination therapy. In some embodiments, a subject can exhibit an overall survival of 18.2 months, 18.3 months, 18.5 months, 18.8 months, 19 months, 19.2 months, 19.5 months, 19.8 months, 20 months, 20.3 months, 20.6 months, 20.8 months, 21 months, 21.2 months, 21.5 months, 21.9 months, 22 months, 22.4 months, 22.6 months, 22.8 months, 23 months, 23.3 months, 23.6 months, 24 months, 25 months, 26 months, 27 months, 27.5 months, 28 months, 28.5 months, 29 months, 29.5 months, 30 months, 30.5 months, or 31 months.

Also provided herein is a method for treating or ameliorating a HER2 positive breast cancer in a subject in need thereof, the method comprising administering to the subject an effective amount of a combination therapy comprising tucatinib, capecitabine, and trastuzumab, wherein the subject exhibits a greater than 30% reduction in the risk of death as compared to a subject administered trastuzumab and capecitabine alone. In some embodiments, the subject exhibits a 34% reduction in the risk of death.

In some embodiments as described herein, following administration of the combination therapy comprising tucatinib, capecitabine, and trastuzumab for twenty-four months, the subject has an estimated overall survival rate of greater than 35%. For example, the subject has an estimated overall survival of 35.4%, 35.5%, 36%, 36.6%, 37%, 37.3%, 38%, 38.6%, 39.7%, 40%, 40.5%, 41%, 42%, 43%, 43.6%, 44%, 44.4%, 45%, 45.8%, 46%, 46.8%, 47%, 47.9%, 48%, 48.2%, 48.8%, 49%, 49.7%, 50%, 50.5%, 51%, 52,4%, 52%, 52.8%, or 53%. In some embodiments, the subject has an estimated overall survival rate of greater than 40%, greater than 44%, greater than 50%, or greater than 52% following administration of combination therapy comprising tucatinib, capecitabine, and trastuzumab for twenty-four months.

In some embodiments as described herein, following administration of the combination therapy comprising tucatinib, capecitabine, and trastuzumab for thirty months, the subject has an estimated overall survival rate of greater than 30%. For example, the subject has an estimated overall survival of 30.7%, 31%, 31.5%, 32%, 32.8%, 33%, 33.8%, 34%, 34.6%, 35.4%, 35.5%, 36%, 36.6%, 37%, 37.3%, 38%, 38.6%, 39.7%, 40%, 40.5%, 41%, 42%, 42.8%, 43%, 43.6%, 44%, 44.4%, 45%, 45.8%, 46%, 46.8%, 47%, 47.9%, 48%, 48.2%, 48.8%, 49%, 49.7%, 50%, 50.5%, 51%, 51.3%, or 52%. In some embodiments, the subject has an estimated overall survival rate of greater than 35%, greater than 40%, greater than 42%, or greater than 50% following administration of combination therapy comprising tucatinib, capecitabine, and trastuzumab for thirty months.

Further provided herein is a methods of treating or ameliorating a brain metastasis in a subject having HER2 positive breast cancer, the method comprising administering to the subject an effective amount of a combination therapy comprising tucatinib, capecitabine, and trastuzumab. In some embodiments, the time to additional intervention (e.g., radiation, surgery, or a combination thereof) for treatment of the brain metastasis in the subject has been increased. In some embodiments, the time to additional intervention is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, and at least 99%. In some embodiments, the time to additional intervention is increased by at least one week, two weeks, three weeks, at least one month, at least two months, at least three months, at least four months, at least five months, at least six months, at least seven months, at least eight months, at least nine months, at least ten months, at least eleven months, at least twelve months, at least eighteen months, and at least twenty-four months. In some embodiments, the need for additional intervention for treatment of the brain metastasis in the subject has been prevented. In some embodiments, the increase in time to additional intervention is compared to a subject administered trastuzumab and capecitabine alone (i.e., a combination of trastuzumab and capecitabine) over the same period of time.

In some embodiments, of the methods or uses or product for uses described herein, response to treatment with a combination of tucatinib, capecitabine, and trastuzumab as described herein, results in prevention of the development of a brain metastasis in the subject (e.g., in a subject that did not previously develop brain metastasis). In some embodiments, of the methods or uses or product for uses described herein, response to treatment with a combination of tucatinib, capecitabine, and trastuzumab as described herein, prevents the development of new brain metastasis (e.g., in a subject previously identified as having brain metastasis). In some embodiments, regression of an existing brain metastasis in the subject has been promoted. In some embodiments, the size of an existing brain metastasis in the subject has been reduced.

Also provided herein is a method for treating or ameliorating a HER2 positive breast cancer in a subject in need thereof, wherein the subject has a brain metastasis, the method comprising administering to the subject an effective amount of a combination therapy comprising tucatinib, capecitabine, and trastuzumab, wherein the subject exhibits a greater than 50% reduction in the risk of disease progression or death as compared to a subject administered trastuzumab and capecitabine alone. In some embodiments, the subject exhibits a 52% reduction in the risk of disease progression or death.

In one aspect, provided herein are methods for treating or ameliorating a HER2 positive breast cancer in a subject in need thereof, wherein the subject has a brain metastasis, the method comprising administering to the subject an effective amount of a combination therapy comprising tucatinib, capecitabine, and trastuzumab, wherein following administration of the combination therapy, the subject exhibits progression-free survival of at least six months following administration of the combination therapy. For example, the subject can exhibit progression-free survival of at least seven months, at least eight months, at least nine months, or at least ten months following administration of the combination therapy. In some embodiments, a subject can exhibit progression-free survival of 6.2 months, 6.4 months, 6.9 months, 7 months, 7.5 months, 7.6 months, 7.7 months, 7.8 months, 7.9 months, 8 months, 8.2 months, 8.5 months, 8.8 months, 9.5 months, 9.8 months, and 10 months following administration of the combination therapy.

In some embodiments as described herein, following administration of the combination therapy comprising tucatinib, capecitabine, and trastuzumab for nine months, the subject having brain metastasis has an estimated progression-free survival rate of greater than 30%. For example, the subject has an estimated progression-free survival of 30.7%, 31%, 31.5%, 32%, 32.8%, 33%, 33.1%, 34%, 34.9%, 35%, 35.5%, 36%, 36.8%, 37%, 37.3%, 38%, 38.6%, 39.7%, 40%, 40.5%, 41%, 42%, 43%, 43.4%, 44%, 44.4%, 45%, 45.8%, 46%, 46.8%, 47%, 47.9%, 48%, 48.2%, 48.8%, 49%, 49.7%, 50%, 50.5%, 51%, 51.5%, or 52%. In some embodiments, the subject has an estimated progression-free survival rate of greater than 40%, greater than 45%, or greater than 50% following administration of combination therapy comprising tucatinib, capecitabine, and trastuzumab for nine months.

In some embodiments as described herein, following administration of the combination therapy comprising tucatinib, capecitabine, and trastuzumab for twelve months, the subject having a brain metastasis has an estimated progression-free survival rate of greater than 15%. For example, the subject has an estimated progression-free survival of 15.8%, 16%, 16.5%, 17%, 18%, 18.8%, 19%, 20%, 22%, 23.3%, 24.9%, 25%, 25.4%, 26%, 26.6%, 27%, 27.4%, 28%, 28.6%, 29%, 29.3%, 30%, 30.7%, 31%, 31.5%, 32%, 32.8%, 33%, 33.1%, 34%, 34.3% or 35%. In some embodiments, the subject has an estimated progression-free survival rate of greater than 20%, greater than 25%, greater than 30%, or greater than 34% following administration of combination therapy comprising tucatinib, capecitabine, and trastuzumab for twelve months.

In some embodiments, the methods provided herein further comprise administration of an anti-diarrheal agent in a subject having a HER2 positive breast cancer and being treated with an effective amount of a combination therapy comprising tucatinib, capecitabine, and trastuzumab. For example, the anti-diarrheal agent can be administered prophylactically (e.g., before or concurrently with administration of the combination therapy comprising tucatinib, capecitabine, and trastuzumab and/or before the subject has symptoms of diarrhea), reactively (e.g., after administration of the combination therapy comprising tucatinib, capecitabine, and trastuzumab and following at least one episode of diarrhea), or a combination thereof. In some embodiments, the anti-diarrheal agent is administered to reduce the severity or incidents of diarrhea, or to prevent diarrhea. In some embodiments, the anti-diarrheal agent is to reduce the likelihood of a subject developing diarrhea.

In some embodiments, the combination therapy and the anti-diarrheal agent are administered sequentially. In some embodiments, the combination therapy and the anti-diarrheal agent are administered concurrently. In some embodiments, the anti-diarrheal agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of diarrhea prior to administration of the anti-diarrheal agent. In other cases, the subject is not exhibiting symptoms of diarrhea prior to administration of the anti-diarrheal agent.

Non-limiting examples of anti-diarrheal agents include loperamide, budesonide (e.g., in combination with loperamide), prophylactic antibiotics (e.g., doxycycline), probiotics, electrolyte replacement solutions, colestipol, colestipol in combination with loperamide, octreotide, crofelemer, TJ14, Bacillus Cereus, calcium aluminosilicate, sulfasalazine, cefpodoxime, elsiglutide, glutamine, codeine, diphenoxylate, atropine, bismuth subsalicylate, diphenoxylate, atropine, attapulgite, activated charcoal, bentonite, saccharomyces boulardii lyo, rifaximin, neomycin, alosetron, octreotide, crofelemer, opium, cholestyramine, and colesevelam.

C. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of tucatinib is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In particular embodiments, a dose of tucatinib is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day). In certain of these embodiments, a dose of tucatinib is 300 mg (e.g., 6×50 mg tablets; or 2×150 mg tablets), administered twice per day.

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering antibodies and/or antibody-drug conjugate of the present disclosure are well known in the art and may be selected by those of ordinary skill in the art. In one embodiment, tucatinib administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib is intravenous injection or infusion. In some embodiments, the route of administration of tucatinib is intravenous infusion. In some embodiments, the route of administration of tucatinib is intravenous injection or infusion. In some embodiments, the tucatinib is intravenous infusion. In some embodiments, the route of administration of tucatinib is oral.

In one embodiment of the methods or uses or product for uses provided herein, tucatinib is administered to the subject daily, twice daily, three times daily or four times daily. In some embodiments, tucatinib is administered to the subject every other day, once about every week or once about every three weeks. In some embodiments, tucatinib is administered to the subject once per day. In some embodiments, tucatinib is administered to the subject twice per day. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally.

D. Capicitabine Dose and Administration

In some embodiments, capecitabine is administered to the subject at a dose based on the body surface area of the subject. In some embodiments, capecitabine is administered to the subject at a dose of about 500 mg/m² to about 1500 mg/m². In some embodiments, capecitabine is administered to the subject at a dose of about 500 mg/m², about 550 mg/m², about 600 mg/m2, about 650 mg/m², about 700 mg/m², about 750 mg/m², about 800 mg/m², about 850 mg/m2, about 900 mg/m², about 950 mg/m², about 1000 mg/m², about 1050 mg/m², about 1100 mg/m2, about 1150 mg/m², about 1200 mg/m², about 1250 mg/m², about 1300 mg/m², about 1350 mg/m², about 1400 mg/m², about 1450 mg/m², or about 1500 mg/m². In some embodiments, capecitabine is administered to the subject at a dose of 500 mg/m² to 1500 mg/m². In some embodiments, capecitabine is administered to the subject at a dose of 500 mg/m², 550 mg/m², 600 mg/m², 650 mg/m², 700 mg/m², 750 mg/m², 800 mg/m², 850 mg/m², 900 mg/m², 950 mg/m², 1000 mg/m², 1050 mg/m², 1100 mg/m², 1150 mg/m², 1200 mg/m², 1250 mg/m², 1300 mg/m², 1350 mg/m², 1400 mg/m², 1450 mg/m², or 1500 mg/m². In some embodiments, capecitabine is administered to the subject daily, twice daily, three times daily or four times daily. In some embodiments, capecitabine is administered to the subject every other day, once about every week or once about every three weeks. In some embodiments, capecitabine is administered to the subject once per day. In some embodiments, capecitabine is administered to the subject twice per day. In some embodiments, capecitabine is administered to the subject twice per day on days 1-14 of a 21 day treatment cycle. In some embodiments, capecitabine is administered to the subject at a dose of about 1000 mg/m² twice per day. In some embodiments, capecitabine is administered to the subject at a dose of 1000 mg/m² twice per day. In some embodiments, capecitabine is administered to the subject at a dose of about 1000 mg/m² twice per day on days 1-14 of a 21 day treatment cycle. In some embodiments, capecitabine is administered to the subject at a dose of 1000 mg/m² twice per day on days 1-14 of a 21 day treatment cycle. In some embodiments, the capecitabine is administered to the subject orally.

In some embodiments, the capecitabine is administered to the subject orally in fixed doses. In certain of these embodiments, the administered dose of capecitabine differs from the exact calculated dose (e.g., by 0-5%, 0-1%, 0-0.5%, 0-0.05%, or 0-0.005%). In some embodiments, capecitabine is prepared and administered according to instructions in the package insert. In some embodiments, capecitabine is administered orally based on instructions provided by medical personnel. In some embodiments, capecitabine is administered with food. In some embodiments, capecitabine is stored according to the package insert.

E. Trastuzumab Dose and Administration

In some embodiments, a dose of trastuzumab is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In some embodiments, a dose of trastuzumab is between about 4 mg and 10 mg per kg of the subject's body weight. In some embodiments, a dose of trastuzumab is between 4 mg and 10 mg per kg of the subject's body weight. In some embodiments, a dose of trastuzumab is about 6 mg per kg of the subject's body weight. In some embodiments, a dose of trastuzumab is about 8 mg per kg of the subject's body weight. In some embodiments, a dose of trastuzumab is about 8 mg per kg of the subject's body weight for the first dose of trastuzumab administered to the subject followed by subsequent doses of about 6 mg per kg of the subject's body weight. In some embodiments, a dose of trastuzumab is 6 mg per kg of the subject's body weight. In some embodiments, a dose of trastuzumab is 8 mg per kg of the subject's body weight. In some embodiments, a dose of trastuzumab is 8 mg per kg of the subject's body weight for the first dose of trastuzumab administered to the subject followed by subsequent doses of 6 mg per kg of the subject's body weight. In other embodiments, a dose of trastuzumab is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of trastuzumab is at least about 100 mg to 500 mg per kg of the subject's body weight (e.g., at least about 100, 125, 150, 175, 200, 225, 250, 275, 300, 325, 350, 375, 400, 425, 450, 475, 500, or more mg per kg of the subject's body weight). In some instances, a dose of trastuzumab is about 6 mg per kg of the subject's body weight. In other instances, a dose of trastuzumab is about 8 mg per kg of the subject's body weight. In some other instances, a dose of trastuzumab is about 20 mg per kg of the subject's body weight. In some embodiments, a dose of trastuzumab comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of trastuzumab. In other embodiments, a dose of trastuzumab comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of trastuzumab. In particular embodiments, a dose of trastuzumab comprises between about 100 mg and 400 mg (e.g., about 100, 125, 150, 175, 200, 225, 250, 275, 300, 325, 350, 375, or 400 mg) of trastuzumab. In some embodiments, a dose of trastuzumab is between about 400 mg and 800 mg. In some embodiments, a dose of trastuzumab is between 400 mg and 800 mg. In some embodiments, a dose of trastuzumab is about 600 mg. In some embodiments, a dose of trastuzumab is 600 mg. As a non-limiting example, when using a dose of 6 mg/kg, a dose fora 50 kg subject will be about 300 mg. As another non-limiting example, when using a dose of 8 mg/kg, a dose for a 50 kg subject will be about 400 mg. In some embodiments, a dose of trastuzumab comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of trastuzumab. In some embodiments, a dose of trastuzumab contains a therapeutically effective amount of trastuzumab. In other embodiments, a dose of trastuzumab contains less than a therapeutically effective amount of trastuzumab (e.g., when multiple doses are given in order to achieve the desired clinical or therapeutic effect). In some embodiments, trastuzumab is administered to the subject once about every 1 to 4 weeks. In certain embodiments, trastuzumab is administered once about every 1 week, once about every 2 weeks, once about every 3 weeks or once about every 4 weeks. In one embodiment, trastuzumab is administered once about every 3 weeks. In some embodiments, trastuzumab is administered to the subject once every 1 to 4 weeks. In certain embodiments, trastuzumab is administered once every 1 week, once about every 2 weeks, once about every 3 weeks or once about every 4 weeks. In one embodiment, trastuzumab is administered once every 3 weeks. In some embodiments, trastuzumab is administered to the subject subcutaneously. In some embodiments, trastuzumab is administered to the subject intravenously. In some embodiments, trastuzumab is administered at a dose of about 600 mg once about every 3 weeks and trastuzumab is administered subcutaneously. In some embodiments, trastuzumab is administered at a dose of 600 mg once every 3 weeks and trastuzumab is administered subcutaneously. In some embodiments, trastuzumab is administered at a dose of about 6 mg/kg once about every 3 weeks and trastuzumab is administered intravenously. In some embodiments, trastuzumab is administered at a dose of about 8 mg/kg once about every 3 weeks and trastuzumab is administered intravenously. In some embodiments, trastuzumab is administered once about every 3 weeks at a dose of about 8 mg/kg for the first dose of trastuzumab administered to the subject followed by subsequent doses of about 6 mg/kg, wherein trastuzumab is administered intravenously. In some embodiments, trastuzumab is administered at a dose of 6 mg/kg once every 3 weeks and trastuzumab is administered intravenously. In some embodiments, trastuzumab is administered at a dose of 8 mg/kg once every 3 weeks and trastuzumab is administered intravenously. In some embodiments, the trastuzumab is administered once every 3 weeks at a dose of 8 mg/kg for the first dose of trastuzumab administered to the subject followed by subsequent doses of 6 mg/kg, wherein trastuzumab is administered intravenously. In some embodiments, trastuzumab is administered to the subject on a 21 day treatment cycle and is administered to the subject once per treatment cycle. In some embodiments, trastuzumab is administered once about every week at a dose of about 2 mg/Kg, wherein trastuzumab is administered intravenously. In some embodiments, trastuzumab is administered once every week at a dose of 2 mg/Kg, wherein trastuzumab is administered intravenously. In certain embodiments (when administration of trastuzumab has been delayed in a 21-day treatment cycle), trastuzumab is administered once about every week at a dose of about 2 mg/kg until resynchronization of the cycle length to 21 days, wherein trastuzumab is administered intravenously. In certain embodiments (when administration of trastuzumab has been delayed in a 21 day treatment cycle), trastuzumab is administered once every week at a dose of 2 mg/kg until resynchronization of the cycle length to 21 days, wherein trastuzumab is administered intravenously.

In some embodiments, trastuzumab is administered once every 21-day treatment cycle at a dose of about 8 mg/kg for the first dose of trastuzumab administered to the subject followed by subsequent doses of about 6 mg/kg, wherein trastuzumab is administered intravenously. In some embodiments, trastuzumab is administered once every 21-day treatment cycle at a dose of 8 mg/kg for the first dose of trastuzumab administered to the subject followed by subsequent doses of 6 mg/kg, wherein trastuzumab is administered intravenously. In some embodiments, trastuzumab is administered at a dose of about 6 mg/kg once every 21-day treatment cycle and trastuzumab is administered intravenously. In some embodiments, trastuzumab is administered at a dose of 6 mg/kg once every 21-day treatment cycle and trastuzumab is administered intravenously. In certain embodiments (when the subject has received trastuzumab within 4 weeks of the first dose), trastuzumab is administered at a dose of about 6 mg/kg once every 21-day treatment cycle and trastuzumab is administered intravenously. In certain embodiments (when the subject has received trastuzumab within 4 weeks of the first dose), trastuzumab is administered at a dose of 6 mg/kg once every 21-day treatment cycle and trastuzumab is administered intravenously.

In some embodiments, trastuzumab is administered at a dose of about 600 mg once every 21-day treatment cycle and trastuzumab is administered subcutaneously. In some embodiments, trastuzumab is administered at a dose of 600 mg once every 21-day treatment cycle and trastuzumab is administered subcutaneously.

In some embodiments, trastuzumab is prepared and administered according to instructions in the package insert. In some embodiments, trastuzumab is administered intravenously or subcutaneously under the direction of medical personnel. In some embodiments, trastuzumab is stored according to the package insert.

F. Combination Therapy

Provided herein are methods of treatment comprising administering to the subject a combination therapy comprising tucatinib, capecitabine and trastuzumab. In some embodiments, the combination therapy consists essentially of tucatinib, capecitabine and trastuzumab. In some embodiments, the combination therapy consists of tucatinib, capecitabine and trastuzumab.

In some embodiments, the tucatinib, capecitabine and trastuzumab are administered to the subject on a 21 day treatment cycle. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally. In some embodiments, capecitabine is administered to the subject twice per day. In some embodiments, capecitabine is administered to the subject twice per day on days 1-14 of a 21 day treatment cycle. In some embodiments, capecitabine is administered to the subject at a dose of about 1000 mg/m² twice per day. In some embodiments, capecitabine is administered to the subject at a dose of 1000 mg/m2 twice per day. In some embodiments, capecitabine is administered to the subject at a dose of about 1000 mg/m² twice per day on days 1-14 of a 21 day treatment cycle. In some embodiments, capecitabine is administered to the subject at a dose of 1000 mg/m² twice per day on days 1-14 of a 21 day treatment cycle. In some embodiments, the capecitabine is administered to the subject orally. In some embodiments, trastuzumab is administered at a dose of about 6 mg/kg once about every 3 weeks and trastuzumab is administered intravenously. In some embodiments, trastuzumab is administered at a dose of about 8 mg/kg once about every 3 weeks and trastuzumab is administered intravenously. In some embodiments, trastuzumab is administered once about every 3 weeks at a dose of about 8 mg/kg for the first dose of trastuzumab administered to the subject followed by subsequent doses of about 6 mg/kg, wherein trastuzumab is administered intravenously. In some embodiments, trastuzumab is administered at a dose of 6 mg/kg once every 3 weeks and trastuzumab is administered intravenously. In some embodiments, trastuzumab is administered at a dose of 8 mg/kg once every 3 weeks and trastuzumab is administered intravenously. In some embodiments, trastuzumab is administered once every 3 weeks at a dose of 8 mg/kg for the first dose of trastuzumab administered to the subject followed by subsequent doses of 6 mg/kg, wherein trastuzumab is administered intravenously. In some embodiments, trastuzumab is administered at a dose of about 2 mg/kg once about every week and trastuzumab is administered intravenously. In some embodiments, trastuzumab is administered at a dose of 2 mg/kg once every week and trastuzumab is administered intravenously. In some embodiments, trastuzumab is administered at a dose of about 600 mg once about every 3 weeks and trastuzumab is administered subcutaneously. In some embodiments, trastuzumab is administered at a dose of 600 mg once every 3 weeks and trastuzumab is administered subcutaneously. In some embodiments, trastuzumab is administered to the subject on a 21 day treatment cycle and is administered to the subject once per treatment cycle.

G. Treatment Outcome

In some embodiments, treating the subject comprises inhibiting breast cancer cell growth, inhibiting breast cancer cell proliferation, inhibiting breast cancer cell migration, inhibiting breast cancer cell invasion, decreasing or eliminating one or more signs or symptoms of breast cancer, reducing the size (e.g., volume) of a breast cancer tumor, reducing the number of breast cancer tumors, reducing the number of breast cancer cells, inducing breast cancer cell necrosis, pyroptosis, oncosis, apoptosis, autophagy, or other cell death, increasing survival time of the subject, or enhancing the therapeutic effects of another drug or therapy.

In some embodiments, treating the subject comprises inhibiting brain mestastasis cell growth, inhibiting brain mestastasis cell proliferation, inhibiting brain mestastasis cell migration, inhibiting brain mestastasis cell invasion, decreasing or eliminating one or more signs or symptoms of a brain mestastasis, reducing the size (e.g., volume) of a brain mestastasis, reducing the number of brain mestastasis, inducing brain mestastasis cell necrosis, pyroptosis, oncosis, apoptosis, autophagy, or other cell death, increasing survival time of the subject, or enhancing the therapeutic effects of another drug or therapy.

In some embodiments, treating the subject as described herein results in a tumor growth inhibition (TGI) index that is between about 10% and 70% (e.g., about 10%, 15%, 20%, 25%, 30%, 35%, 40%, 45%, 50%, 55%, 60%, 65%, or 70%). Preferably, treating the subject results in a TGI index that is at least about 70% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%). More preferably, treating the subject results in a TGI index that is at least about 85% (e.g., about 85%, 86%, 87%, 88%, 89%, 90%, 91%, 92%, 93%, 94%, 95%, 96%, 97%, 98%, 99%, or 100%). Even more preferably, treating the subject results in a TGI index that is at least about 95% (e.g., about 95%, 96%, 97%, 98%, 99%, or 100%). Most preferably, treating the subject results in a TGI index that is about 100% or more (e.g., about 100%, 101%, 102%, 103%,104%, 105%, 106%, 107%, 108%, 109%, 110%, 111%, 112%, 113%, 114%, 115%, 116%, 117%, 118%, 119%, 120%, 125%, 130%, 135%, 140%, 145%, 150%, or more).

In particular embodiments, treating the subject with tucatinib, capecitabine and trastuzumab results in a TGI index that is greater than the TGI index that is observed when tucatinib, capecitabine or trastuzumab is used alone. In some instances, treating the subject results in a TGI index that is greater than the TGI index that is observed when tucatinib is used alone. In other instances, treating the subject results in a TGI index that is greater than the TGI index that is observed when capecitabine is used alone. In other instances, treating the subject results in a TGI index that is greater than the TGI index that is observed when trastuzumab is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that is observed when tucatinib, capecitabine or trastuzumab is used alone.

In some embodiments, the combination of the tucatinib, capecitabine and trastuzumab is synergistic. In particular embodiments, with respect to the synergistic combination, treating the subject results in a TGI index that is greater than the TGI index that would be expected if the combination of tucatinib, capecitabine and trastuzumab produced an additive effect. In some instances, the TGI index observed when a combination of tucatinib, capecitabine and trastuzumab is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 5500 60%, 65%, 70%, 75%, or 80% greater than the TGI index that would be expected if the combination of tucatinib, capecitabine and trastuzumab produced an additive effect.

In one aspect, a method of treating cancer with tucatinib as described herein results in an improvement in one or more therapeutic effects in the subject after administration of a combination of tucatinib, capecitabine, and trastuzumab relative to a baseline. In some embodiments, the one or more therapeutic effects is the size of the tumor derived from the breast cancer, the objective response rate, the duration of response, the time to response, progression free survival, overall survival, or any combination thereof. In one embodiment, the one or more therapeutic effects is the size of the tumor derived from the breast cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with a combination of tucatinib, capecitabine, and trastuzumab as described herein may include the following criteria (RECIST Criteria 1.1):

| | Category | Criteria |
|---|---|---|
| Based on target | Complete Response | Disappearance of all target lesions. Any pathological |
| lesions | (CR) | lymph nodes must have reduction in short axis to <10 |
| | | mm. |
| | Partial Response | ≥30% decrease in the sum of the longest diameter (LD) |
| | (PR) | of target lesions, taking as reference the baseline sum of |
| | | LDs. |
| | Stable Disease (SD) | Neither sufficient shrinkage to qualify for PR nor |
| | | sufficient increase to qualify for PD, taking as reference |
| | | the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with a combination of tucatinib, capecitabine, and trastuzumab as described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with a combination of tucatinib, capecitabine, and trastuzumab described herein is assessed by measuring the size of a tumor derived from the cancer (e.g., breast cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the combination of tucatinib, capecitabine, and trastuzumab. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the combination of tucatinib, capecitabine, and trastuzumab. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In some embodiments, the size of a tumor derived from a breast cancer is measured by mammography, sonography or magnetic resonance imaging (MRI). See Gruber et. al., 2013, BMC Cancer. 13:328.

In one embodiment of the methods or uses or product for uses provided described herein, response to treatment with a combination of tucatinib, capecitabine, and trastuzumab described herein, promotes regression of a tumor derived from the cancer (e.g., breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In some embodiments, regression of a tumor is determined by mammography, sonography or magnetic resonance imaging (MRI). See Gruber et. al., 2013, BMC Cancer. 13:328.

In one embodiment of the methods or uses or product for uses described herein, response to treatment with a combination of tucatinib, capecitabine, and trastuzumab as described herein is assessed by measuring the duration of response to a combination of tucatinib, capecitabine, and trastuzumab after administration of the combination of tucatinib, capecitabine, and trastuzumab. In some embodiments, the duration of response to a combination of tucatinib, capecitabine, and trastuzumab is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the combination of tucatinib, capecitabine, and trastuzumab. In some embodiments, the duration of response to a combination of tucatinib, capecitabine, and trastuzumab is at least about 6 months after administration of the combination of tucatinib, capecitabine, and trastuzumab. In some embodiments, the duration of response to a combination of tucatinib, capecitabine, and trastuzumab is at least about one year after administration of the combination of tucatinib, capecitabine, and trastuzumab. In some embodiments, the duration of response to a combination of tucatinib, capecitabine, and trastuzumab is at least about two years after administration of the combination of tucatinib, capecitabine, and trastuzumab. In some embodiments, the duration of response to a combination of tucatinib, capecitabine, and trastuzumab is at least about three years after administration of the combination of tucatinib, capecitabine, and trastuzumab. In some embodiments, the duration of response to a combination of tucatinib, capecitabine, and trastuzumab is at least about four years after administration of the combination of tucatinib, capecitabine, and trastuzumab. In some embodiments, the duration of response to a combination of tucatinib, capecitabine, and trastuzumab is at least about five years after administration of the combination of tucatinib, capecitabine, and trastuzumab. In some embodiments, the duration of response to a combination of tucatinib, capecitabine, and trastuzumab is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the combination of tucatinib, capecitabine, and trastuzumab. In some embodiments, the duration of response to a combination of tucatinib, capecitabine, and trastuzumab is at least 6 months after administration of the combination of tucatinib, capecitabine, and trastuzumab. In some embodiments, the duration of response to a combination of tucatinib, capecitabine, and trastuzumab is at least one year after administration of the combination of tucatinib, capecitabine, and trastuzumab. In some embodiments, the duration of response to a combination of tucatinib, capecitabine, and trastuzumab is at least two years after administration of the combination of tucatinib, capecitabine, and trastuzumab. In some embodiments, the duration of response to a combination of tucatinib, capecitabine, and trastuzumab is at least three years after administration of the combination of tucatinib, capecitabine, and trastuzumab. In some embodiments, the duration of response to a combination of tucatinib, capecitabine, and trastuzumab is at least four years after administration of the combination of tucatinib, capecitabine, and trastuzumab. In some embodiments, the duration of response to a combination of tucatinib, capecitabine, and trastuzumab is at least five years after administration of the combination of tucatinib, capecitabine, and trastuzumab.

In one embodiment of the methods or uses or product for uses provided herein, response to treatment with a combination of tucatinib, capecitabine, and trastuzumab described herein is assessed by measuring the size of a brain metastasis derived from the cancer (e.g., breast cancer). In one embodiment, the size of a brain metastasis derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the brain metastasis derived from the cancer before administration of the combination of tucatinib, capecitabine, and trastuzumab. In one embodiment, the size of a brain metastasis derived from the cancer is reduced by at least about 10%-80%. In one embodiment, the size of a brain metastasis derived from the cancer is reduced by at least about 20%-80%. In one embodiment, the size of a brain metastasis derived from the cancer is reduced by at least about 30%-80%. In one embodiment, the size of a brain metastasis derived from the cancer is reduced by at least about 40%-80%. In one embodiment, the size of a brain metastasis derived from the cancer is reduced by at least about 50%-80%. In one embodiment, the size of a brain metastasis derived from the cancer is reduced by at least about 60%-80%. In one embodiment, the size of a brain metastasis derived from the cancer is reduced by at least about 70%-80%. In one embodiment, the size of a brain metastasis derived from the cancer is reduced by at least about 80%. In one embodiment, the size of a brain metastasis derived from the cancer is reduced by at least about 85%. In one embodiment, the size of a brain metastasis derived from the cancer is reduced by at least about 90%. In one embodiment, the size of a brain metastasis derived from the cancer is reduced by at least about 95%. In one embodiment, the size of a brain metastasis derived from the cancer is reduced by at least about 98%. In one embodiment, the size of a brain metastasis derived from the cancer is reduced by at least about 99%. In one embodiment, the size of a brain metastasis derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the brain metastasis derived from the cancer before administration of the combination of tucatinib, capecitabine, and trastuzumab. In one embodiment, the size of a brain metastasis derived from the cancer is reduced by at least 10%-80%. In one embodiment, the size of a brain metastasis derived from the cancer is reduced by at least 20%-80%. In one embodiment, the size of a brain metastasis derived from the cancer is reduced by at least 30%-80%. In one embodiment, the size of a brain metastasis derived from the cancer is reduced by at least 40%-80%. In one embodiment, the size of a brain metastasis derived from the cancer is reduced by at least 50%-80%. In one embodiment, the size of a brain metastasis derived from the cancer is reduced by at least 60%-80%. In one embodiment, the size of a brain metastasis derived from the cancer is reduced by at least 70%-80%. In one embodiment, the size of a brain metastasis derived from the cancer is reduced by at least 80%. In one embodiment, the size of a brain metastasis derived from the cancer is reduced by at least 85%. In one embodiment, the size of a brain metastasis derived from the cancer is reduced by at least 90%. In one embodiment, the size of a brain metastasis derived from the cancer is reduced by at least 95%. In one embodiment, the size of a brain metastasis derived from the cancer is reduced by at least 98%. In one embodiment, the size of a brain metastasis derived from the cancer is reduced by at least 99%. In one embodiment, the size of a brain metastasis derived from the cancer is reduced by 100%.

In one embodiment of the methods or uses or product for uses provided described herein, response to treatment with a combination of tucatinib, capecitabine, and trastuzumab described herein, promotes regression of a brain metastasis derived from the cancer (e.g., breast cancer). In one embodiment, a brain metastasis derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the brain metastasis derived from the cancer before administration of the combination of tucatinib, capecitabine, and trastuzumab described herein. In one embodiment, a brain metastasis derived from the cancer regresses by at least about 10% to about 80%. In one embodiment, a brain metastasis derived from the cancer regresses by at least about 20% to about 80%. In one embodiment, a brain metastasis derived from the cancer regresses by at least about 30% to about 80%. In one embodiment, a brain metastasis derived from the cancer regresses by at least about 40% to about 80%. In one embodiment, a brain metastasis derived from the cancer regresses by at least about 50% to about 80%. In one embodiment, a brain metastasis derived from the cancer regresses by at least about 60% to about 80%. In one embodiment, a brain metastasis derived from the cancer regresses by at least about 70% to about 80%. In one embodiment, a brain metastasis derived from the cancer regresses by at least about 80%. In one embodiment, a brain metastasis derived from the cancer regresses by at least about 85%. In one embodiment, a brain metastasis derived from the cancer regresses by at least about 90%. In one embodiment, a brain metastasis derived from the cancer regresses by at least about 95%. In one embodiment, a brain metastasis derived from the cancer regresses by at least about 98%. In one embodiment, a brain metastasis derived from the cancer regresses by at least about 99%. In one embodiment, a brain metastasis derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the brain metastasis derived from the cancer before administration of the combination of tucatinib, capecitabine, and trastuzumab as described herein. In one embodiment, a brain metastasis derived from the cancer regresses by at least 10% to 80%. In one embodiment, a brain metastasis derived from the cancer regresses by at least 20% to 80%. In one embodiment, a brain metastasis derived from the cancer regresses by at least 30% to 80%. In one embodiment, a brain metastasis derived from the cancer regresses by at least 40% to 80%. In one embodiment, a brain metastasis derived from the cancer regresses by at least 50% to 80%. In one embodiment, a brain metastasis derived from the cancer regresses by at least 60% to 80%. In one embodiment, a brain metastasis derived from the cancer regresses by at least 70% to 80%. In one embodiment, a brain metastasis derived from the cancer regresses by at least 80%. In one embodiment, a brain metastasis derived from the cancer regresses by at least 85%. In one embodiment, a brain metastasis derived from the cancer regresses by at least 90%. In one embodiment, a brain metastasis derived from the cancer regresses by at least 95%. In one embodiment, a brain metastasis derived from the cancer regresses by at least 98%. In one embodiment, a brain metastasis derived from the cancer regresses by at least 99%. In one embodiment, a brain metastasis derived from the cancer regresses by 100%.

In some embodiments, the size, progression, regression, and/or response of a brain metastasis to administration of a combination of tucatinib, capecitabine, and trastuzumab as described herein is determined using one or more of the RANO-BM criteria. See, for example, Lin, N. U. et al. The Lancet 16 (June 2015): e270-e278.

H. Compositions

In another aspect, the present disclosure provides a pharmaceutical composition comprising tucatinib and a pharmaceutically acceptable carrier. In another aspect, the present disclosure provides a pharmaceutical composition comprising capecitabine and a pharmaceutically acceptable carrier. In another aspect, the present disclosure provides a pharmaceutical composition comprising trastuzumab and a pharmaceutically acceptable carrier. In another aspect, the present disclosure provides a pharmaceutical composition comprising tucatinib, capecitabine, and a pharmaceutically acceptable carrier. In another aspect, the present disclosure provides a pharmaceutical composition comprising tucatinib, trastuzumab, and a pharmaceutically acceptable carrier. In another aspect, the present disclosure provides a pharmaceutical composition comprising capecitabine, trastuzumab, and a pharmaceutically acceptable carrier. In another aspect, the present disclosure provides a pharmaceutical composition comprising tucatinib, capecitabine, trastuzumab, and a pharmaceutically acceptable carrier.

In some embodiments, tucatinib is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, tucatinib is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, tucatinib is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, tucatinib is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, trastuzumab is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, trastuzumab is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, trastuzumab is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, trastuzumab is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, capecitabine is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, capecitabine is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, capecitabine is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, capecitabine is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

The pharmaceutical compositions of the present disclosure may be prepared by any of the methods well-known in the art of pharmacy. Pharmaceutically acceptable carriers suitable for use with the present disclosure include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present disclosure can include a combination of drugs (e.g., tucatinib, capecitabine, and trastuzumab), or any pharmaceutically acceptable salts thereof, as active ingredients and a pharmaceutically acceptable carrier or excipient or diluent. A pharmaceutical composition may optionally contain other therapeutic ingredients.

The compositions (e.g., comprising tucatinib, capecitabine, trastuzumab, or a combination thereof) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present disclosure may be administered intratumorally.

Compositions for pulmonary administration include, but are not limited to, dry powder compositions consisting of the powder of a compound described herein (e.g., tucatinib, capecitabine, trastuzumab, or a combination thereof), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., tucatinib, capecitabine, trastuzumab, or a combination thereof) and the powder of a suitable carrier or excipient. The compositions for systemic administration can be represented by, but not limited to, tablets, capsules, pills, syrups, solutions, and suspensions.

In some embodiments, the compositions (e.g., tucatinib, capecitabine, trastuzumab, or a combination thereof) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPPCD, CD, glycerol, maltose, mannitol, and saccharose.

Pharmaceutical compositions or medicaments for use in the present disclosure can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., tucatinib, capecitabine, trastuzumab, or a combination thereof) can be made as implants, oily injections, or as particulate systems. For a broad overview of delivery systems see Banga, A. J., THERAPEUTIC PEPTIDES AND PROTEINS: FORMULATION, PROCESSING, AND DELIVERY SYSTEMS, Technomic Publishing Company, Inc., Lancaster, PA, (1995), which is incorporated herein by reference. Particulate systems include microspheres, microparticles, microcapsules, nanocapsules, nanospheres, and nanoparticles.

Polymers can be used for ion-controlled release of compositions of the present disclosure. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has been shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, PA (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and U.S. Pat. Nos. 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

For oral administration of a combination of tucatinib, capecitabine, and/or trastuzumab, a pharmaceutical composition or a medicament can take the form of, for example, a tablet or a capsule prepared by conventional means with a pharmaceutically acceptable excipient. The present disclosure provides tablets and gelatin capsules comprising tucatinib, capecitabine, trastuzumab, or a combination thereof, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol; for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of tucatinib, capecitabine, trastuzumab, or a combination thereof include creams, ointments, sprays, lotions, and patches. The pharmaceutical composition can, however, be formulated for any type of administration, e.g., intradermal, subdermal, intravenous, intramuscular, subcutaneous, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., tucatinib, capecitabine, trastuzumab, or a combination thereof) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising tucatinib, capecitabine, trastuzumab, or a combination thereof) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising tucatinib, capecitabine, trastuzumab, or a combination thereof) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

### Pharmaceutical Compositions of Tucatinib

In some embodiments, a pharmaceutical composition comprising tucatinib and a pharmaceutically acceptable carrier is provided herein, wherein the pharmaceutical composition comprises a solid dispersion of tucatinib.

The solid dispersions are generally prepared by dissolving the drug substance and the dispersion polymer in a suitable solvent to form a feed solution, and then the feed solution may be spray dried to form the solid dispersion (and remove the solvent). Spray drying is a known process. Spray drying is generally performed by dissolving tucatinib and the dispersion polymer in a suitable solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput. Although the solid dispersion are preferably prepared by conventional spray drying techniques, other techniques known in the art may be used, such as melt extrusion, freeze drying, rotary evaporation, drum drying or other solvent removal processes.

In some embodiments, a process of preparing a solid dispersion is provided, comprising: (a) dissolving tucatinib and a dispersion polymer in a suitable solvent; and (b) evaporating the solvent to form the solid dispersion. In certain embodiments, the evaporation of the solvent in step (b) is performed by spray drying, melt extrusion, freeze drying, rotary evaporation, drum drying or other solvent removal processes.

In certain embodiments, the dispersion polymer is selected from PVP-VA, methylacrylic acid methyl methacrylate copolymer, HPMCP, CAP, HPMCAS and HPMC and mixtures thereof. In certain embodiments, the dispersion polymer is selected from PVP-VA, methylacrylic acid methyl methacrylate copolymer, HPMCP, CAP, HPMCAS and HPMC. In certain embodiments, the dispersion polymer is selected from PVP-VA, Eudragit® L100, HPMCP H-55, CAP, HPMCAS Grade M, HPMC and mixtures thereof. In certain embodiments, the dispersion polymer is selected from PVP-VA, Eudragit® L100, HPMCP H-55, CAP, HPMCAS Grade M and HPMC.

In certain embodiments, the dispersion polymer is selected from PVP-VA, methylacrylic acid methyl methacrylate copolymer, HPMCP, CAP and HPMCAS, and mixtures thereof. In certain embodiments, the dispersion polymer is selected from PVP-VA, methylacrylic acid methyl methacrylate copolymer, HPMCP, CAP and HPMCAS. In certain embodiments, the dispersion polymer is selected from PVP-VA, Eudragit® L100, HPMCP H-55, CAP and HPMCAS Grade M, and mixtures thereof. In certain embodiments, the dispersion polymer is selected from PVP-VA, Eudragit® L100, HPMCP H-55, CAP and HPMCAS Grade M.

In certain embodiments, the dispersion polymer is selected from PVP-VA, methylacrylic acid methyl methacrylate copolymer, HPMCP, CAP and HPMC, and mixtures thereof. In certain embodiments, the dispersion polymer is selected from PVP-VA, methylacrylic acid methyl methacrylate copolymer, HPMCP, CAP and HPMC. In certain embodiments, the dispersion polymer is selected from PVP-VA, Eudragit® L100, HPMCP H-55, CAP and HPMC, and mixtures thereof. In certain embodiments, the dispersion polymer is selected from PVP-VA, Eudragit® L100, HPMCP H-55, CAP and HPMC

In certain embodiments, the dispersion polymer is selected from PVP-VA, methylacrylic acid methyl methacrylate copolymer, HPMCP and CAP, and mixtures thereof. In certain embodiments, the dispersion polymer is selected from PVP-VA, methylacrylic acid methyl methacrylate copolymer, HPMCP and CAP. In certain embodiments, the dispersion polymer is selected from PVP-VA, Eudragit® L100, HPMCP H-55 and CAP, and mixtures thereof. In certain embodiments, the dispersion polymer is selected from PVP-VA, Eudragit® L100, HPMCP H-55 and CAP. In certain embodiments, the dispersion polymer is PVP-VA.

In certain embodiments, the dispersion polymer is methylacrylic acid methyl methacrylate copolymer. In certain embodiments, the dispersion polymer is Eudragit®. In certain embodiments, the dispersion polymer is Eudragit® L100.

In certain embodiments, the dispersion polymer is HPMCP. In certain embodiments, the dispersion polymer is HPMCP H-55.

In certain embodiments, the dispersion polymer is CAP.

In certain embodiments, the dispersion polymer is HPMCAS. In certain embodiments, the dispersion polymer is HPMCAS Grade M.

In certain embodiments, the dispersion polymer is preferably neutral or basic.

In certain embodiments, the dispersion polymer is selected from PVP-VA and HPMC. In certain embodiments, the dispersion polymer is HPMC.

Suitable solvents are a solvent or mixture of solvents in which both tucatinib and the dispersion polymer have adequate solubility (solubility greater than 1 mg/mL). A mixture of solvents may be used if each component of the solid dispersion (i.e., tucatinib and dispersion polymer) require different solvents to obtain the desired solubility. The solvent may be volatile with a boiling point of 150° C. or less. In addition, the solvent should have relatively low toxicity and be removed from the dispersion to a level that is acceptable to The International Committee on Harmonization (“ICH”) guidelines. Removal of solvent to this level may require a subsequent processing step, such as tray drying. Examples of suitable solvents include, but are not limited to, alcohols, such as methanol (“MeOH”), ethanol (“EtOH”), n-propanol, isopropanol (“IPA”) and butanol; ketones, such as acetone, methyl ethyl ketone (“MEK”) and methyl isobutyl ketone; esters, such as ethyl acetate (“EA”) and propyl acetate; and various other solvents, such as tetrahydrofuran (“THF”), acetonitrile (“ACN”), methylene chloride, toluene and 1,1,1-trichloroethane. Lower volatility solvents, such as dimethyl acetate or dimethylsulfoxide (“DMSO”), may be used. Mixtures of solvents with water may also be used, so long as the polymer and tucatinib are sufficiently soluble to make the spray drying process practicable. Generally, due to the hydrophobic nature of low solubility drugs, non-aqueous solvents may be used, meaning the solvent comprises less than about 10 weight % water.

In certain embodiments, the suitable solvent is selected from MeOH and THF, and mixtures thereof. In certain embodiments, the suitable solvent is MeOH:THF solvent system of about 1:3. In certain embodiments, the suitable solvent is a 1:3 MeOH:THF solvent system.

In certain embodiments, the suitable solvent is selected from MeOH, THE and water, and mixtures thereof. In certain embodiments, the suitable solvent is selected from MeOH, THE and water. In certain embodiments, the suitable solvent is a THF:MeOH:water solvent system of about 80:10:10. In certain embodiments, the suitable solvent is a 80:10:10 THF:MeOH:water solvent system. In certain embodiments, the suitable solvent is a THF:MeOH:water solvent system of about 82:8:10. In certain embodiments, the suitable solvent is a 82:8:10 THF:MeOH:water solvent system. In certain embodiments, the suitable solvent is a THF:MeOH:water solvent system of about 82.2:8.2:9.6. In certain embodiments, the suitable solvent is a 82.2:8.2:9.6 THF:MeOH:water solvent system.

In certain embodiments, the amount of tucatinib in the solid dispersion ranges from about 0.1% to about 70% by weight relative to the dispersion polymer. In certain embodiments, the amount of tucatinib in the solid dispersion ranges from 0.1% to 70% by weight relative to the dispersion polymer.

In certain embodiments, the amount of tucatinib in the solid dispersion ranges from about 1% to about 60% by weight relative to the dispersion polymer. In certain embodiments, the amount of tucatinib in the solid dispersion ranges from 1% to 60% by weight relative to the dispersion polymer.

In certain embodiments, the amount of tucatinib in the solid dispersion ranges from about 5% to about 60% by weight relative to the dispersion polymer. In certain embodiments, the amount of tucatinib in the solid dispersion ranges from 5% to 60% by weight relative to the dispersion polymer.

In certain embodiments, the amount of tucatinib in the solid dispersion ranges from about 55% to about 65% by weight relative to the dispersion polymer. In certain embodiments, the amount of tucatinib in the solid dispersion ranges from 55% to 65% by weight relative to the dispersion polymer. In certain embodiments, the amount of tucatinib in the solid dispersion is about 60% by weight relative to the dispersion polymer. In certain embodiments, the amount of tucatinib in the solid dispersion is 60% by weight relative to the dispersion polymer.

In certain embodiments, the amount of tucatinib in the solid dispersion ranges from about 25% to about 35% by weight relative to the dispersion polymer. In certain embodiments, the amount of tucatinib in the solid dispersion ranges from 25% to 35% by weight relative to the dispersion polymer. In certain embodiments, the amount of tucatinib in the solid dispersion is about 30% by weight relative to the dispersion polymer. In certain embodiments, the amount of tucatinib in the solid dispersion is 30% by weight relative to the dispersion polymer.

In certain embodiments, the amount of tucatinib in the solid dispersion ranges from about 45% to about 55% by weight relative to the dispersion polymer. In certain embodiments, the amount of tucatinib in the solid dispersion ranges from 45% to 55% by weight relative to the dispersion polymer. In certain embodiments, the amount of tucatinib in the solid dispersion is about 50% by weight relative to the dispersion polymer. In certain embodiments, the amount of tucatinib in the solid dispersion is 50% by weight relative to the dispersion polymer.

In certain embodiments, the solid dispersion is an amorphous solid dispersion.

Another embodiment provides a pharmaceutical composition comprising a solid dispersion of tucatinib and a dispersion polymer, and a carrier or excipient.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

The pharmaceutical compositions may also include one or more additional components, such as buffers, dispersion agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug, i.e., a compound described herein or pharmaceutical composition thereof, or aid in the manufacturing of the pharmaceutical product, i.e., medicament (see Ansel; Gennaro; and Rowe above). The components of the pharmaceutical composition should be pharmaceutically acceptable.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a binder/diluent.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a binder/diluent.

Certain embodiments provide a pharmaceutical composition comprising: (a) about 25 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 25 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 40 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 40 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a filler.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a filler.

Certain embodiments provide a pharmaceutical composition comprising: (a) about 25 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 25 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

Certain embodiments provide a pharmaceutical composition comprising: (a) about 40 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 40 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

In certain embodiments, the osmogen is selected from NaCl and KCl, and mixtures thereof.

In certain embodiments, the lubricant is magnesium stearate.

In certain embodiments, the glidant is colloidal silicon dioxide.

In certain embodiments, the binder/diluent is microcrystalline cellulose. In certain embodiments, the binder/diluent acts as both a binder and a diluent.

In certain embodiments, the binder is microcrystalline cellulose.

In certain embodiments, the diluent is microcrystalline cellulose.

In certain embodiments, the filler is lactose.

In certain embodiments, the disintegrant is selected from crospovidone and sodium bicarbonate (NaHCO₃), and mixtures thereof. In certain embodiments, the disintegrant is selected from crospovidone and sodium bicarbonate. In certain embodiments, the disintegrant is sodium bicarbonate. In certain embodiments, the disintegrant is crospovidone.

In certain embodiments, the composition contains sodium bicarbonate. tucatinib may slowly degrade, through hydrolysis or other means, to a carbamate impurity:

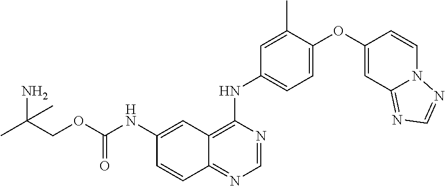 [SMILES: Cc1cc(Nc2ncnc3ccc(NC(=O)OCC(C)(C)N)cc23)ccc1Oc1ccn2ncnc2c1]

N Sodium bicarbonate helps to slow the degradation to the carbamate impurity. Sodium bicarbonate also helps to provide consistent tablet disintegration when the tablets are exposed to different humidities.

Certain embodiments provide a pharmaceutical composition comprising: (a) tucatinib; and (b) sodium bicarbonate.

Certain embodiments provide a pharmaceutical composition comprising: (a) about 1 to about 70 weight % of a solid dispersion of tucatinib; and (b) about 0.1 to about 30 weight % sodium bicarbonate.

In certain embodiments, the pharmaceutical composition comprises: (a) 1 to 70 weight % of a solid dispersion of tucatinib; and (b) 0.1 to 30 weight % sodium bicarbonate.

Certain embodiments provide a pharmaceutical composition comprising: (a) about 1 to about 70 weight % of a solid dispersion of tucatinib; (b) about 0.1 to about 30 weight % sodium bicarbonate; and (c) the remaining weight is other pharmaceutically acceptable excipients and carriers.

In certain embodiments, the pharmaceutical composition comprises: (a) 1 to 70 weight % of a solid dispersion of tucatinib; (b) 0.1 to 30 weight % sodium bicarbonate; and (c) the remaining weight is other pharmaceutically acceptable excipients and carriers.

Certain embodiments provide a pharmaceutical composition comprising: (a) about 25 to about 60 weight % of a solid dispersion of tucatinib; and (b) about 1 to about 15 weight % of sodium bicarbonate.

In certain embodiments, the pharmaceutical composition comprises: (a) 25 to 60 weight % of a solid dispersion of tucatinib; and (b) 1 to 15 weight % of sodium bicarbonate.

Certain embodiments provide a pharmaceutical composition comprising: (a) about 25 to about 60 weight % of a solid dispersion of tucatinib; (b) about 1 to about 15 weight % of sodium bicarbonate; and (c) the remaining weight is other pharmaceutically acceptable excipients and carriers.

In certain embodiments, the pharmaceutical composition comprises: (a) 25 to 60 weight % of a solid dispersion of tucatinib; (b) 1 to 15 weight % of sodium bicarbonate; and (c) the remaining weight is other pharmaceutically acceptable excipients and carriers.

Certain embodiments provide a pharmaceutical composition comprising: (a) about 40 to about 60 weight % of a solid dispersion of tucatinib; and (b) about 1 to about 15 weight % of sodium bicarbonate.

In certain embodiments, the pharmaceutical composition comprises: (a) 40 to 60 weight % of a solid dispersion of tucatinib; and (b) 1 to 15 weight % of sodium bicarbonate.

Certain embodiments provide a pharmaceutical composition comprising: (a) about 40 to about 60 weight % of a solid dispersion of tucatinib; (b) about 1 to about 15 weight % of sodium bicarbonate; (c) the remaining weight is other pharmaceutically acceptable excipients and carriers.

In certain embodiments, the pharmaceutical composition comprises: (a) 40 to 60 weight % of a solid dispersion of tucatinib; (b) 1 to 15 weight % of sodium bicarbonate; (c) the remaining weight is other pharmaceutically acceptable excipients and carriers.

Certain embodiments provide a pharmaceutical composition comprising: (a) about 40 to about 60 weight % of a solid dispersion of tucatinib; (b) about 5 to about 15 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof, (d) about 0.1 to about 3 weight % of a glidant which is colloidal silicon dioxide; (e) about 0.1 to about 3 weight % of a lubricant which is magnesium stearate; and (f) about 10 to about 25 weight % of a binder/diluent which is microcrystalline cellulose.

In certain embodiments, the pharmaceutical composition comprises: (a) 40 to 60 weight % of a solid dispersion of tucatinib; (b) 5 to 15 weight % of a disintegrant which is selected from the group of crospovidone, sodium bicarbonate (NaHCO₃), and mixtures thereof, (c) 15 to 25 weight % of an osmogen which is selected from the group consisting of NaCl, KCl, and mixtures thereof, (d) 0.1 to 3 weight % of a glidant which is colloidal silicon dioxide; (e) 0.1 to 3 weight % of a lubricant which is magnesium stearate; and (f) 10 to 25 weight % of a binder/diluent which is microcrystalline cellulose.

Certain embodiments provide a pharmaceutical composition comprising: (a) about 40 to about 60 weight % of a solid dispersion of tucatinib; (b) about 1 to about 10 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof, (d) about 0.1 to about 3 weight % of a glidant which is colloidal silicon dioxide; (e) about 0.1 to about 3 weight % of a lubricant which is magnesium stearate; and (f) about 10 to about 25 weight % of a filler which is lactose.

In certain embodiments, the pharmaceutical composition comprises: (a) 40 to 60 weight % of a solid dispersion of tucatinib; (b) 1 to 10 weight % of a disintegrant which is selected from the group of crospovidone, sodium bicarbonate (NaHC₃), and mixtures thereof, (c) 15 to 25 weight 0 of an osmogen n osmogen which is selected from the group consisting of NaCl, KCl, and mixtures thereof; (d) 0.1 to 3 weight % of a glidant which is colloidal silicon dioxide; (e) 0.1 to 3 weight 0 of a lubricant which is magnesium stearate; and (f) 10 to 25 weight % of a filler which is lactose.

In certain embodiments, the pharmaceutical composition is selected from the group consisting of:

| Function | Ingredient | % of Blend |
|---|---|---|
| API | Solid dispersion of tucatinib | about 50 |
| Disintegrant | Crospovidone - | about 6 |
| | Polyplasdone ® | |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline | about 19.25 |
| | cellulose - Avicel ® | |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - | about 6 |
| | Polyplasdone ® | |
| Disintegrant | NaHCO₃ | about 3 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline | about 16.25 |
| | cellulose - Avicel ® | |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - | about 6 |
| | Polyplasdone ® | |
| Osmogen | NaCl | about 10.625 |
| Osmogen | KCl | about 10.625 |
| Filler | Lactose | about 21.25 |
| Glidant | Colloidal Silicon Dioxide | about 0.5 |
| Lubricant | Magnesium Stearate | aabout 0.25 |

Extragranular

| | | |
|---|---|---|
| Glidant | Colloidal Silicon Dioxide | about 0.5 |
| Lubricant | Magnesium Stearate | about 0.25 |

In certain embodiments, the pharmaceutical composition is selected from the group consisting of:

| Function | Ingredient | % of Blend |
|---|---|---|
| API | Solid dispersion of tucatinib | 50 |
| Disintegrant | Crospovidone - | 6 |
| | Polyplasdone ® | |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline | 19.25 |
| | cellulose - Avicel ® | |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - | 6 |
| | Polyplasdone ® | |
| Disintegrant | NaHCO₃ | 3 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline | 16.25 |
| | cellulose - Avicel ® | |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - | 6 |
| | Polyplasdone ® | |
| Osmogen | NaCl | 10.625 |
| Osmogen | KCl | 10.625 |
| Filler | Lactose | 21.25 |
| Glidant | Colloidal Silicon Dioxide | 0.5 |
| Lubricant | Magnesium Stearate | 0.25 |

Extragranular

| | | |
|---|---|---|
| Glidant | Colloidal Silicon Dioxide | 0.5 |
| Lubricant | Magnesium Stearate | 0.25 |

The pharmaceutical composition preferably contains a therapeutically effective amount of tucatinib. However, in some embodiments, each individual dose contains a portion of a therapeutically effective amount of tucatinib, such that multiple doses of the composition may be required (for example, two or more tablets are required for a therapeutically effective amount). Thus, in this application when it states that the pharmaceutical composition contains a therapeutically effective amount it means that the composition may be one dose (for example, one tablet) or multiple doses (for example, two tablets). In certain embodiments, the pharmaceutical composition contains between 1 and 500 mg of tucatinib.

In certain embodiments, the pharmaceutical composition contains between about 25 and about 400 mg of tucatinib. In certain embodiments, the pharmaceutical composition contains between 25 and 400 mg of tucatinib.

In certain embodiments, the pharmaceutical composition contains between about 25 and about 100 mg (e.g., about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg) of tucatinib. In certain embodiments, the pharmaceutical composition contains between 25 and 100 mg (e.g., 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg) of tucatinib. In certain embodiments, the pharmaceutical composition contains between about 25 and about 75 mg of tucatinib. In certain embodiments, the pharmaceutical composition contains between 25 and 75 mg of tucatinib. In certain embodiments, the pharmaceutical composition contains about 50 mg of tucatinib. In certain particular embodiments, the pharmaceutical composition contains 50 mg of tucatinib. In certain of the foregoing embodiments, the pharmaceutical composition is formulated as a tablet. As a non-limiting example, the pharmaceutical composition is formulated as a tablet and contains 50 mg of tucatinib.

In certain embodiments, the pharmaceutical composition contains between about 100 and about 300 mg (e.g., about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg) of tucatinib. In certain embodiments, the pharmaceutical composition contains between 100 and 300 mg (e.g., 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg) of tucatinib. In certain embodiments, the pharmaceutical composition contains between about 100 and about 200 mg of tucatinib. In certain embodiments, the pharmaceutical composition contains between 100 and 200 mg of tucatinib. In certain embodiments, the pharmaceutical composition contains between about 125 and about 175 mg of tucatinib. In certain embodiments, the pharmaceutical composition contains between 125 and 175 mg of tucatinib. In certain embodiments, the pharmaceutical composition contains about 150 mg of tucatinib. In certain particular embodiments, the pharmaceutical composition contains 150 mg of tucatinib. In certain of the foregoing embodiments, the pharmaceutical composition is formulated as a tablet. As a non-limiting example, the pharmaceutical composition is formulated as a tablet and contains 150 mg of tucatinib.

The pharmaceutical compositions described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), ocular, vaginal, intraperitoneal, intrapulmonary and intranasal. If parenteral administration is desired, the compositions will be sterile and in a solution or suspension form suitable for injection or infusion.

The compounds may be administered in any convenient administrative form, e.g., tablets, powders, capsules, dispersions, suspensions, syrups, sprays, suppositories, gels, emulsions, patches, etc.

The pharmaceutical compositions described herein are typically administered orally. The pharmaceutical compositions described herein are typically administered as a tablet, caplet, hard or soft gelatin capsule, pill, granules or a suspension.

Additional examples of pharmaceutical compositions of tucatinib and methods of preparation thereof are described in U.S. Pat. No. 9,457,093, which is incorporated by reference herein in its entirety.

The pharmaceutical compositions described herein may comprise one or more polymorphs of tucatinib. Exemplary polymorphs of tucatinib and methods of preparation thereof are described in U.S. Pat. No. 9,168,254, which is incorporated by reference herein in its entirety.

In some embodiments, the pharmaceutical composition comprises amorphous tucatinib. In certain embodiments, tucatinib in the pharmaceutical composition is substantially amorphous (e.g., at least 80%, at least 85%, at least 90%, or at least 95% amorphous).

In some embodiments, the pharmaceutical composition comprises a crystalline polymorph of tucatinib. In certain embodiments, tucatinib in the pharmaceutical composition is substantially crystalline (e.g., at least 80%, at least 85%, at least 90%, or at least 95% crystalline).

In certain embodiments, the pharmaceutical composition comprises polymorph Form A of tucatinib as described in U.S. Pat. No. 9,168,254. In certain embodiments, tucatinib in the pharmaceutical composition is substantially in Form A (e.g., at least 80%, at least 85%, at least 90%, or at least 95% Form A).

In certain embodiments, the pharmaceutical composition comprises polymorph Form B of tucatinib as described in U.S. Pat. No. 9,168,254. In certain embodiments, tucatinib in the pharmaceutical composition is substantially in Form B (e.g., at least 80%, at least 85%, at least 90%, or at least 95% Form B).

In certain embodiments, the pharmaceutical composition comprises polymorph Form C of tucatinib as described in U.S. Pat. No. 9,168,254. In certain embodiments, tucatinib in the pharmaceutical composition is substantially in Form C (e.g., at least 80%, at least 85%, at least 90%, or at least 95% Form C).

In certain embodiments, the pharmaceutical composition comprises polymorph Form D of tucatinib as described in U.S. Pat. No. 9,168,254. In certain embodiments, tucatinib in the pharmaceutical composition is substantially in Form D (e.g., at least 80%, at least 85%, at least 90%, or at least 95% Form D).

In certain embodiments, the pharmaceutical composition comprises polymorph Form E of tucatinib as described in U.S. Pat. No. 9,168,254. In certain embodiments, tucatinib in the pharmaceutical composition is substantially in Form E (e.g., at least 80%, at least 85%, at least 90%, or at least 95% Form E).

In certain embodiments, the pharmaceutical composition comprises polymorph Form F of tucatinib as described in U.S. Pat. No. 9,168,254. In certain embodiments, tucatinib in the pharmaceutical composition is substantially in Form F (e.g., at least 80%, at least 85%, at least 90%, or at least 95% Form F).

In certain embodiments, the pharmaceutical composition comprises polymorph Form G of tucatinib as described in U.S. Pat. No. 9,168,254. In certain embodiments, tucatinib in the pharmaceutical composition is substantially in Form G (e.g., at least 80%, at least 85%, at least 90%, or at least 95% Form G).

In certain embodiments, the pharmaceutical composition comprises polymorph Form H of tucatinib as described in U.S. Pat. No. 9,168,254. In certain embodiments, tucatinib in the pharmaceutical composition is substantially in Form H (e.g., at least 80%, at least 85%, at least 90%, or at least 95% Form H).

In certain embodiments, the pharmaceutical composition comprises polymorph Form I of tucatinib as described in U.S. Pat. No. 9,168,254. In certain embodiments, tucatinib in the pharmaceutical composition is substantially in Form I (e.g., at least 80%, at least 85%, at least 90%, or at least 95% Form I).

In certain embodiments, the pharmaceutical composition comprises polymorph Form J of tucatinib as described in U.S. Pat. No. 9,168,254. In certain embodiments, tucatinib in the pharmaceutical composition is substantially in Form J (e.g., at least 80%, at least 85%, at least 90%, or at least 95% Form J).

In certain embodiments, the pharmaceutical composition comprises polymorph Form K of tucatinib as described in U.S. Pat. No. 9,168,254. In certain embodiments, tucatinib in the pharmaceutical composition is substantially in Form K (e.g., at least 80%, at least 85%, at least 90%, or at least 95% Form K).

In certain embodiments, the pharmaceutical composition comprises polymorph Form L of tucatinib as described in U.S. Pat. No. 9,168,254. In certain embodiments, tucatinib in the pharmaceutical composition is substantially in Form L (e.g., at least 80%, at least 85%, at least 90%, or at least 95% Form L).

In certain embodiments, the pharmaceutical composition comprises polymorph Form M of tucatinib as described in U.S. Pat. No. 9,168,254. In certain embodiments, tucatinib in the pharmaceutical composition is substantially in Form M (e.g., at least 80%, at least 85%, at least 90%, or at least 95% Form M).

In certain embodiments, the pharmaceutical composition comprises polymorph Form N of tucatinib as described in U.S. Pat. No. 9,168,254. In certain embodiments, tucatinib in the pharmaceutical composition is substantially in Form N (e.g., at least 80%, at least 85%, at least 90%, or at least 95% Form N).

In certain embodiments, the pharmaceutical composition comprises polymorph Form O of tucatinib as described in U.S. Pat. No. 9,168,254. In certain embodiments, tucatinib in the pharmaceutical composition is substantially in Form O (e.g., at least 80%, at least 85%, at least 90%, or at least 95% Form O).

In certain embodiments, the pharmaceutical composition comprises polymorph Form P of tucatinib as described in U.S. Pat. No. 9,168,254. In certain embodiments, tucatinib in the pharmaceutical composition is substantially in Form P (e.g., at least 80%, at least 85%, at least 90%, or at least 95% Form P).

### I. Articles of Manufacture and Kits

In another aspect, the present disclosure provides an article of manufacture or kit for treating or ameliorating the effects of breast cancer in a subject, the article of manufacture or kit comprising a pharmaceutical composition of the present disclosure (e.g., a pharmaceutical composition comprising tucatinib, capecitabine, trastuzumab, or a combination thereof).

The articles of manufacture or kits are suitable for treating or ameliorating the effects of breast cancers, particularly HER2 positive and/or metastatic breast cancers. In some embodiments, the cancer is an advanced cancer. In some other embodiments, the cancer is a drug-resistant cancer. In some instances, the cancer is a multidrug-resistant cancer.

Materials and reagents to carry out the various methods of the present disclosure can be provided in articles of manufacture or kits to facilitate execution of the methods. As used herein, the term “kit” includes a combination of articles that facilitates a process, assay, analysis, or manipulation. In particular, kits of the present disclosure find utility in a wide range of applications including, for example, diagnostics, prognostics, therapy, and the like.

Articles of manufacture or kits can contain chemical reagents as well as other components. In addition, the articles of manufacture or kits of the present disclosure can include, without limitation, instructions to the user, apparatus and reagents for administering combinations of tucatinib, capecitabine and trastuzumab or pharmaceutical compositions thereof, sample tubes, holders, trays, racks, dishes, plates, solutions, buffers, or other chemical reagents. Articles of manufacture or kits of the present disclosure can also be packaged for convenient storage and safe shipping, for example, in a box having a lid.

### III. Exemplary Embodiments

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Phase 2 Randomized, Double-Blinded, Controlled Study of Tucatinib Vs. Placebo in Combination with Capecitabine and Trastuzumab in Patients with Pretreated Unresectable Locally Advanced or Metastatic HER2+ Breast Carcinoma

This example describes a double-blinded study of tucatinib or placebo in combination with capecitabine and trastuzumab is carried out in patients with unresectable locally advanced or metastatic HER2+ breast cancer who have had prior treatment with trastuzumab, pertuzumab and T-DM1.

### Background and Rationale

### HER2+ Breast Cancer

Breast cancer is the most common form of cancer in women worldwide (1), and the second leading cause of cancer-related death in the United States (2). Approximately 20% of breast cancers overexpress the human epidermal growth factor receptor 2 (HER2) (3,4). HER2 is a transmembrane tyrosine kinase receptor that mediates cell growth, differentiation, and survival. Tumors that overexpress HER2 are more aggressive and historically have been associated with poorer overall survival (OS) compared to HER2 negative cancers (5).

The introduction of HER2-targeted therapy using either antibody-based therapy or a small molecule tyrosine kinase inhibitor (TKI) has led to significant and ongoing improvements in disease-free survival (DFS), progression-free survival (PFS), and OS in both the adjuvant and metastatic settings (6-9). Trastuzumab, a humanized anti-HER2 antibody, remains the backbone of treatment in the adjuvant and first-line metastatic settings, usually in combination with a taxane. Anti-HER2 therapy in combination with cytotoxic chemotherapy allows for concurrent treatment with agents having two different mechanisms of action, leading to greater efficacy than with either agent alone (6, 10, 11).

Despite the improvements in outcomes for early stage HER2+ breast cancer, up to a quarter of all patients treated with anti-HER2 therapy in the adjuvant setting relapse. The development of new HER2 targeted therapies such as pertuzumab and T-DM1 (ado-trastuzumab emtansine or trastuzumab emtansine) for metastatic HER2+ breast cancer has led to a meaningful prolongation in the median survival of these patients; however, essentially all patients in the metastatic setting ultimately progress. Treatment failures may result from primary or acquired resistance to HER2 blockade (12-15). There is evidence that dual targeting of HER2, either through combination of 2 different HER2-targeted antibodies or through use of an antibody-based therapy such as trastuzumab and a TKI, can lead to further improvements in efficacy in metastatic disease (8, 16). In particular, combination of a small molecule TKI with an antibody-based therapy may be effective, as it may help overcome resistance to antibody-mediated inhibition through utilization of an alternative mechanism of receptor inhibition. Lapatinib, a dual epidermal growth factor receptor (EGFR)/IER2 oral TKI, has been shown to have increased activity in combination with trastuzumab compared to lapatinib alone, even when given to patients who have previously progressed on prior trastuzumab-based therapy (17,18). Use of lapatinib, however, has been limited by the anti-EGFR/human epidermal growth factor receptor 1 (HER1) activity of the drug, which results in toxicities such as rash, diarrhea, and fatigue. There is therefore a need for a more selective small molecule inhibitor of HER2 that could be combined with other anti-HER2 therapies to improve clinical outcomes.

The current standard of care for patients with HER2+ metastatic disease consists of treatment with pertuzumab plus trastuzumab and a taxane as first-line treatment for metastatic disease, followed by T-DM1 in second line (4,19). Treatment options for patients who progress after treatment with both pertuzumab and T-DM1 remain relatively limited. Patients are generally treated with a continuation of anti-HER2 therapy (in the form of trastuzumab or lapatinib) in combination with cytotoxic chemotherapy, such as capecitabine. Combined HER2 therapy with trastuzumab and lapatinib can also be considered. However, no single regimen is considered the standard of care in this setting and better options for these patients are needed.

### Brain Metastases in HER2+ Breast Cancer

Perhaps the greatest unmet medical need in the post-trastuzumab era is treatment and prevention of brain metastases. Recent data suggest that the incidence of first relapse occurring in the brain is increasing in patients who have received trastuzumab-based adjuvant therapy (20), and approximately 30-50% of HER2+ patients with metastatic disease will develop brain metastases (20-22). The increasing prevalence of brain metastases in HER2+ breast cancer patients may be due to several factors. First, HER2+ breast cancer appears to display tropism for the brain. Second, with better control of non-CNS disease, patients may be living longer allowing brain metastases to become more of a critical clinical issue. Finally, the brain may represent a sanctuary site for HER2+ disease as large molecules, such as trastuzumab, do not penetrate the blood-brain barrier (23).

Treatment options for brain metastases are limited. There is no specific systemic treatment regimen approved for brain metastases, and treatment currently relies heavily on the use of local therapies such as whole brain radiation therapy (WBRT), stereotactic radiation (SRS), or surgery. Patients may also receive chemotherapy alone, or capecitabine and either lapatinib or trastuzumab, although brain response rates are generally modest (24, 25). The development of HER2-targeted systemic therapies with clinical benefit in both brain and non-CNS sites of disease could lead to improved clinical outcomes, both by improving overall PFS and OS as well as by avoiding or delaying the use of radiation therapy and its associated toxicities, including neurocognitive impairment.

### Study Design

After signing informed consent and meeting all eligibility criteria, patients were randomized in a 2:1 ratio to receive tucatinib or placebo in combination with capecitabine and trastuzumab. Approved trastuzumab biosimilars (intravenous or subcutaneous formulations) might also be used in the study as an alternative to trastuzumab.

Randomization of patients for the trial was made using a dynamic hierarchical randomization schema. Rosenberger, William F., and John M. Lachin. “Chapter 7.” Randomization in Clinical Trials Theory and Practice. Hoboken, NJ: John Wiley & Sons, 2016. Stratification factors included presence or history of treated or untreated brain metastases or brain lesions of equivocal significance (yes/no), Eastern Cooperative Oncology Group Performance Status (ECOG PS) (0 vs. 1), and region of world (US vs Canada vs Rest of World). Stratification for presence of brain metastases was based upon medical history and investigator assessment of screening contrast brain MRI. Patients who had prior brain metastases (treated or untreated) or unequivocal presence of brain metastases on screening MRI were considered a “Yes” for stratification purposes, and subsequent efficacy assessments. Patients with no prior history of brain metastases and lesions of equivocal significance on screening contrast brain MRI were also considered a “Yes” for purposes of stratification and follow-up.

Treatment was administered in cycles of 21 days each. Tucatinib (300 mg) or placebo was given by mouth (PO) twice daily (BID). If necessary, the tucatinib or placebo dose was reduced to 250 mg, 200 mg, or even 150 mg PO BID to avoid side effects.

Capecitabine was given at 1000 mg/m² PO BID on Days 1-14 of each 21-day cycle.

Trastuzumab was given as a loading dose of 8 mg/kg IV. Following an IV loading dose of trastuzumab, 6 mg/kg of trastuzumab was administered once every 21 days, except in specific circumstances where it might be given weekly to compensate for modifications in treatment schedule. A loading dose of trastuzumab was not given to patients who had received trastuzumab within 4 weeks of the beginning of the trial's first cycle. These patients received trastuzumab at 6 mg/kg each cycle, including Cycle 1. Trastuzumab might also be given on a weekly basis at 2 mg/kg IV q 7 days, but only in the circumstance that trastuzumab infusion has been delayed, and weekly infusions were required to resynchronize the cycle length to 21 days.

Alternatively, trastuzumab was administered as a subcutaneous dose, given as a fixed dose of 600 mg once every 3 weeks. Subcutaneous trastuzumab did not require a loading dose nor was a weekly schedule available for the intravenous formulation. Patients were permitted to crossover from IV trastuzumab to subcutaneous trastuzumab.

Dose modifications of tucatinib or placebo and capecitabine were allowed. Dose holding or discontinuation of tucatinib or placebo, capecitabine, and trastuzumab was also allowed as needed for patient safety. Patients who discontinued either capecitabine or trastuzumab (but not both) might remain on study treatment. Patients who discontinued tucatinib or placebo, or both capecitabine and trastuzumab were not allowed to remain on study treatment.

Treatment continued until unacceptable toxicity, disease progression, withdrawal of consent, or study closure. In patients with isolated progression in the brain and stable systemic disease, local therapy to the brain might be administered.

Patients might then continue on study treatment for clinical benefit after a PFS event in brain with medical monitor approval. Because this approach approximated common practice off-study in this clinical scenario, the duration of treatment after the brain-only progression was analyzed as an exploratory objective. After discontinuing study treatment, patients might receive further care as determined by their physician. In the absence of clear evidence of disease progression (per RECIST 1.1), development of CNS symptoms or radiographic changes thought to pose potential immediate risk to patient, all efforts were made to continue treatment until unequivocal evidence of radiologic progression occurs, as defined in RECIST 1.1. No crossover from placebo to tucatinib was allowed. It was planned that patients would be followed for efficacy endpoints after treatment discontinuation until the protocol specified number of events (both PFS and OS) were observed.

Patients were assessed throughout the study for safety. Safety assessments including physical exam, collection of AEs, and laboratory assessments were performed at a minimum of once every three weeks throughout study treatment and 30 days after the last dose of study drugs. Laboratory assessments were performed locally at sites. Cardiac ejection fraction was assessed by MUGA scan or ECHO at screening and at least once every 12 weeks thereafter.

Laboratory assessments included the following tests: calcium, magnesium, inorganic phosphorus, uric acid, total protein, lactate dehydrogenase (LDH), albumin, blood urea nitrogen (BUN), creatinine, bicarbonate, glucose, potassium, chloride, and sodium. Liver function tests (LFT) included the following: AST/SGOT, ALT/SGPT, total bilirubin, and alkaline phosphatase. The hematology panel included the following tests: complete blood count (CBC) with differential, hemoglobin, hematocrit (Hct), and platelets. The coagulation panel included the following tests: INR, prothrombin time (PT), and aPTT. The urinalysis included (but not limited to) the following tests: color, appearance, pH, protein, glucose, ketones, and blood.

Contrast brain MRI was performed at baseline in all patients regardless of prior history of brain metastases. Efficacy assessments included measurement of all known sites of metastatic or locally advanced unresectable disease (including at a minimum the chest, abdomen, and pelvis) by high quality spiral contrast CT, PET/CT (if high quality CT scan included) and/or MRI scan as appropriate, as well as appropriate imaging of any other known sites of disease (e.g., skin lesion photography, bone imaging) at baseline, every 6 weeks for the first 24 weeks, and then every 9 weeks thereafter. Repeat contrast brain MRI was required on this same schedule only in those patients with prior history of brain metastases, brain metastases found at screening, or brain lesions of equivocal significance found at screening. Contrast brain MRI might also be performed in patients without known brain metastases if there was clinical suspicion of new brain lesions. Additional imaging such as nuclear medicine bone scan or other scans might be performed at the discretion of the investigator. Treatment decisions were made based upon investigator assessment of radiologic scans. All patients underwent a repeat contrast MRI of the brain within 30 days of the end of treatment, unless a contrast MRI of the brain had already been performed within 30 days or there was prior documentation of progression in the brain on study. If study treatment was discontinued for reasons other than disease progression (per RECIST 1.1), patients continued to be followed for progressive disease including submission of subsequent imaging so as to define PFS. All patients in the study continued to be followed for OS after completion of study treatment. Patients who were randomized but did not receive treatment were also followed for PFS and OS.

For patients who underwent local therapy to brain metastases incidentally found on screening contrast brain MRI, and then continued onto study treatment, the performance of a repeat contrast MRI after completion of local therapy was as follows: For patients who received brain radiotherapy during the screening period, the original baseline contrast brain MRI served as the baseline for comparison for further response assessments. For patients who underwent surgical resection of brain metastases during the screening period, a post-operative contrast brain MRI served as the baseline. Contrast brain MRIs were then done per the protocol defined schedule of events even if all lesions in the brain received local treatment after the screening MRI. However, treated lesions were not considered as target lesions. Treatment changes which might mimic progression were taken into account, and patients with possible “pseudo-progression” continued on study until unequivocal evidence of radiographic or clinical progression is present.

Pharmacokinetic assessments of peak and trough levels of tucatinib and metabolite drug levels were performed. Blood samples were also taken for possible evaluation of potential biomarkers of response, including circulating tumor DNA (ctDNA). Individual (patient) plasma tucatinib concentrations at each sampling time were listed; corresponding summary statistics at each sampling time were also calculated. Plasma tucatinib vs. time profiles (with concentrations on both a log and linear scale) were plotted for each patient; corresponding summary time plots were likewise constructed. The ratio of the metabolite ONT-993 to the parent drug tucatinib was listed and summarized at each sampling time.

Safety monitoring was performed throughout the study on a blinded basis. An independent Data Monitoring Committee (DMC) regularly reviewed all relevant safety data including (but not limited to) deaths, discontinuations, dose reductions, AEs, serious adverse events (SAEs), and cases of progressive disease within 6 weeks of study entry (blinded and unblinded) as outlined in a separate DMC charter. Ad hoc meetings of the DMC might be held upon the request of the sponsor or DMC.

Health-related quality of life and health care economics were assessed by use of the EQ-5D-5L quality of life instrument and collection of health care resource utilization data.

The primary efficacy endpoint was progression-free survival (PFS), defined as the time from randomization to centrally-reviewed documented disease progression or death from any cause (as determined by BICR per RECIST 1.1), whichever occurs earlier. The analysis of the primary endpoint was performed using the first 480 randomized patients in the ITT population. For the primary endpoint of centrally-reviewed PFS in the study as a whole, the two treatment groups were compared using a stratified log-rank test. The p-value for this test was calculated using a re-randomization procedure (Rosenberger, William F., and John M. Lachin. “Chapter 7.” Randomization in Clinical Trials Theory and Practice. Hoboken, NJ: John Wiley & Sons, 2016) to reflect the dynamic allocation used in randomization: known history of treated or untreated brain metastases (yes/no); ECOG PS (0 vs. 1); and region of world. All randomized patients were included in the primary analysis. Patients who were alive and had not progressed at the time of the analysis was censored at the time of their last tumor assessment that was a CR, PR, non-CR/non-PD or SD. Details of the censoring scheme or the primary analysis of PFS are described in the SAP.

Kaplan-Meier methodology was used to estimate the PFS time curves. The median PFS and its 95% confidence interval (CI) was provided for two treatment arms. A Cox proportional-hazards model taking into account the stratification factors, was used to estimate the hazard ratio (HR) and its 95% CI.

For patients who received non-protocol specified anti-cancer therapy (NPT) prior to documented PD, details of the censoring scheme and sensitivity analyses to be performed for the primary endpoint was described in the statistical analysis plan for this study.

Secondary efficacy endpoints were progression-free survival in patients with brain metastases, duration of overall survival, investigator-assessed PFS, objective response rate, clinical benefit rate, and duration of response (for responsive patients).

Exploratory efficacy evaluations were also performed using the bi-compartmental tumor assessment method. In this analysis, progression (independent central review) with non-CNS disease was evaluated per the Response Evaluation Criteria In Solid Tumors (RECIST) 1.1 criteria and CNS disease was evaluated per the Response Assessment in NeuroOncology—Brain Metastases (RANO-BM) criteria. HER2 and other mutations were explored as possible biomarkers of response though the use of descriptive subgroup analyses of the primary and secondary endpoints.

Follow-up for PFS continued for 12 months after the last patient was randomized. Follow-up for OS continued until a sufficient number of events have been recorded to have 90% power to test the effect of treatment on OS. As the median survival for the control arm (i.e., administration of capecitabine and trastuzumab alone) might range from 15 to 24 months, the primary analysis for OS took place approximately 1-2+ years after the primary analysis of PFS.

### Endpoints

### Primary Endpoint

PFS, defined as the time from randomization to documented disease progression (as determined by BICR per RECIST 1.1), or death from any cause, whichever occurred first.

### Secondary Endpoints

Efficacy endpoints included: PFS in patients with brain metastases at baseline using RECIST 1.1 based on BICR; OS; PFS, defined as the time from randomization to investigator-assessed documented disease progression (per RECIST 1.1), or death from any cause, whichever occurred first; ORR (RECIST 1.1) as determined by BICR as well as the investigator; DOR (RECIST 1.1) as determined by BICR as well as the investigator; and CBR (RECIST 1.1) as determined by BICR as well as the investigator.

Safety endpoints included: adverse events (AEs); clinical laboratory assessments; vital signs and other relevant safety variables; frequency of dose holding, dose reductions, and discontinuations of capecitabine; frequency of dose holding, dose reductions, and discontinuations of tucatinib; and frequency of dose holding and discontinuations of trastuzumab.

Pharmacokinetics endpoints included plasma concentrations of tucatinib and metabolite.

Health economics and outcome endpoints included: cumulative incidence of health resource utilization, including, but not limited to, length of stay, hospitalizations, ED visits; and health-related quality of life/health status using the EQ-5D-5L instrument.

### Exploratory Endpoints

Exploratory endpoints included: PFS (per RANO-BM using the bi-compartmental tumor assessment method (non-brain disease being evaluated per RECIST 1.1 and CNS disease being evaluated per RANO-BM)); non-CNS PFS per RECIST 1.1 in patients who continue on study treatment for clinical benefit following development of and local treatment for first CNS progression; ORR (using bi-compartmental tumor assessment method per RANO-BM by independent central review); duration of response (per RANO-BM bi-compartmental tumor assessment method by independent central review); time to brain progression (per RANO-BM by independent central review); CBR (per RANO-BM bi-compartmental tumor assessment method by independent central review); presence of HER2 mutations or other mutations as potential biomarkers of response; and time to additional intervention (surgery or radiation) for brain metastases.

### Selection and Withdrawal of Patients

### Inclusion Criteria

In order to be eligible for the study, patients must meet the criteria described below.

(1) Patients must have histologically confirmed HER2+ breast carcinoma, with HER2+ defined by ISH or FISH or IHC methodology. Tissue blocks or slides must be submitted to confirm HER2 positivity (using ISH or FISH) by a sponsor-designated central laboratory prior to randomization. Centrally confirmed HER2 results (either IHC, ISH, or FISH) from a previous study could be used to determine eligibility for this study with approval from the sponsor.

(2) Patients must have received previous treatment with trastuzumab, pertuzumab, and T-DM1.

(3) Patients must have progression of unresectable locally advanced or metastatic breast cancer after last systemic therapy (as confirmed by investigator), or be intolerant of last systemic therapy.

(4) Patients must have measurable or non-measureable disease assessable by RECIST 1.1.

(5) Patients must be at least 18 years of age at time of consent.

(6) Patients must have ECOG PS 0 or 1.

(7) Patients must have a life expectancy of at least 6 months, in the opinion of the investigator.

(8) Patients must have adequate hepatic function as defined by a total bilirubin ≤1.5×ULN, except for patients with known Gilbert's disease, who may enroll if the conjugated bilirubin is ≤1.5×ULN; and transaminases AST/SGOT and ALT/SGPT ≤2.5×ULN (≤5×ULN if liver metastases were present).

(9) Patients must have adequate baseline hematologic parameters as defined by ANC ≥1.5×10³/μL; platelet count ≥100×10³/μL (patients with stable platelet count from 75-100×10³/μL might be included with approval from medical monitor); hemoglobin ≥9 g/dL; and in patients transfused before study entry, transfusion must be ≥14 days prior to start of therapy to establish adequate hematologic parameters independent from transfusion support.

(10) Patients must have creatinine clearance ≥50 mL/min as calculated per institutional guidelines or, in patients ≤45 kg in weight, serum creatinine within institutional normal limits.

(11) Patients must have INR and aPTT ≤1.5×ULN unless on medication known to alter INR and aPTT. Patient use of warfarin and other coumarin derivatives were prohibited.

(12) Patients must have LVEF ≥50% as assessed by ECHO or MUGA scan documented within 4 weeks prior to first dose of study treatment.

(13) If a patient was a female of childbearing potential, the patient must have a negative result of a serum or urine pregnancy test performed within 7 days prior to first dose of study treatment. A woman was considered of childbearing potential (i.e., fertile) following menarche and until becoming post-menopausal unless permanently sterile. Permanent sterilization methods included hysterectomy, bilateral salpingectomy, and bilateral oophorectomy. A postmenopausal state was defined as no menses for 12 months without an alternative medical cause. Postmenopausal patients with known β-HCG secreting tumors might be eligible when βHCG-based urine or serum pregnancy tests yielded false positive if they met the definition of postmenopausal state and had a negative uterine ultrasound.

(14) Women of childbearing potential (as defined above) and men with partners of childbearing potential must agree to use a highly effective birth control method, i.e., methods that achieved a failure rate of less than 1% per year when used consistently and correctly. Such methods included: combined (estrogen and progestogen containing) hormonal contraception associated with inhibition of ovulation (oral, intravaginal, or transdermal); progestogen-only hormonal contraception associated with inhibition of ovulation (oral, injectable, or implantable); intrauterine device; intrauterine hormone-releasing system; bilateral tubal occlusion/ligation; vasectomized partner; or sexual abstinence. Male patients with partners of childbearing potential must use barrier contraception. All study patients were instructed to practice effective contraception, as described above, starting from the signing of informed consent until 7 months after the last dose of study medication or investigational medicinal product.

(15) Patients must provide signed informed consent per a consent document that had been approved by an IRB/IEC prior to initiation of any study-related tests or procedures that were not part of standard-of-care for the patient's disease.

(16) Patients must be willing and able to comply with study procedures.

(17) For CNS inclusion, based on screening contrast brain MRI, patients must have one of the criteria described: (i) no evidence of brain metastases; (ii) untreated brain metastases not needing immediate local therapy (for patients with untreated CNS lesions >2.0 cm on screening contrast brain MRI, discussion with and approval from the medical monitor was required prior to enrollment); or (iii) had previously treated brain metastases.

Brain metastases previously treated with local therapy might either be stable since treatment or might have progressed since prior local CNS therapy, provided that there was no clinical indication for immediate re-treatment with local therapy in the opinion of the investigator.

Patients treated with CNS local therapy for newly identified lesions found on contrast brain MRI performed during screening for this study might be eligible to enroll if all of the following criteria were met: time since WBRT was ≥21 days prior to first dose of treatment, time since SRS was ≥7 days prior to first dose of treatment, or time since surgical resection was ≥28 days; and other sites of evaluable disease (by RECIST 1.1) were present.

Relevant records of any CNS treatment must be available to allow for classification of target and non-target lesions.

### Exclusion Criteria

Patients were excluded from the study for any of the reasons described below.

(1) Patient had previously been treated with lapatinib within 12 months of starting study treatment (except in cases where lapatinib was given for ≤21 days and was discontinued for reasons other than disease progression or severe toxicity); or neratinib, afatinib, or other investigational HER2/EGFR or HER2 TKI at any time previously.

(2) Patient had previously been treated with capecitabine (or other fluoropyrimidine [e.g., 5-fluorouracil]) for metastatic disease (except in cases where capecitabine was given for ≤21 days and was discontinued for reasons other than disease progression or severe toxicity). Patients who had received capecitabine for adjuvant or neoadjuvant treatment at least 12 months prior to starting study treatment were eligible.

(3) Patient had a history of exposure to the following cumulative doses of anthracyclines: doxorubicin (>360 mg/m²), epirubicin (>720 mg/m²), mitoxantrone (>120 mg/m²), idarubicin (>90 mg/m²), or liposomal doxorubicin (e.g. Doxil, Caelyx, Myocet) >550 mg/m²).

(4) Patient had a history of allergic reactions to trastuzumab, capecitabine, or compounds chemically or biologically similar to tucatinib, except for Grade 1 or 2 infusion related reactions to trastuzumab that were successfully managed, or known allergy to one of the excipients in the study drugs.

(5) Patient had received treatment with any systemic anti-cancer therapy (including hormonal therapy), non-CNS radiation, or experimental agent ≤3 weeks of first dose of study treatment or were currently participating in another interventional clinical trial. An exception for the washout of hormonal therapies was GnRH agonists used for ovarian suppression in premenopausal women, which were permitted concomitant medications.

(6) Patient had any toxicity related to prior cancer therapies that had not resolved to ≤Grade 1, with the following exceptions: alopecia and neuropathy (which must have resolved to ≤Grade 2); CHF (which must have been ≤Grade 1 in severity at the time of occurrence, and must have resolved completely); and anemia (which must have resolved to ≤Grade 2).

(7) Patient had clinically significant cardiopulmonary disease such as: ventricular arrhythmia requiring therapy; uncontrolled hypertension (defined as persistent systolic blood pressure >150 mm Hg and/or diastolic blood pressure >100 mm Hg on antihypertensive medications); any history of symptomatic CHF; severe dyspnea at rest (CTCAE Grade 3 or above) due to complications of advanced malignancy; hypoxia requiring supplementary oxygen therapy (except when oxygen therapy was needed only for obstructive sleep apnea); presence of ≥Grade 2 QTc prolongation on screening ECG; conditions potentially resulting in drug-induced prolongation of the QT interval or torsade de pointes, such as congenital or acquired long QT syndrome, a family history of sudden death, a history of previous drug induced QT prolongation, or a current use of medications with known and accepted associated risk of QT prolongation (see row “Accepted Association” in Table 13 below).

(8) Patient had a known myocardial infarction or unstable angina within 6 months prior to first dose of study treatment.

(9) Patient was a known carrier of Hepatitis B or Hepatitis C or had other known chronic liver disease.

(10) Patient was known to be positive for HIV.

(11) Patient was pregnant, breastfeeding, or planning a pregnancy.

(12) Patient required therapy with warfarin or other coumarin derivatives (non-coumarin anticoagulants were allowed).

(13) Patient had an inability to swallow pills or significant gastrointestinal disease which would preclude the adequate oral absorption of medications.

(14) Patient had used a strong CYP3A4 or CYP2C8 inhibitor within 5 half-lives of the inhibitor, or had used a strong CYP3A4 or CYP2C8 inducer within 5 days prior to first dose of study treatment (see Tables 10 and 11 at the end of this example).

(15) Patient had a known dihydropyrimidine dehydrogenase deficiency.

(16) Patient was unable for any reason to undergo contrast MRI of the brain.

(17) Patient had any other medical, social, or psychosocial factors that, in the opinion of the investigator, could impact safety or compliance with study procedures.

(18) Patient had evidence within 2 years of the start of study treatment of another malignancy that required systemic treatment.

For CNS exclusion, based on screening brain MRI, patients must not have any of the following:

(19) Patient might not have any untreated brain lesions >2.0 cm in size, unless discussed with medical monitor and approval for enrollment was given.

(20) Patient might not have ongoing use of systemic corticosteroids for control of symptoms of brain metastases at a total daily dose of >2 mg of dexamethasone (or equivalent). However, patients on a chronic stable dose of ≤2 mg total daily of dexamethasone (or equivalent) might be eligible with discussion and approval by the medical monitor.

(21) Patient might not have any brain lesion thought to require immediate local therapy, including, but not limited to, a lesion in an anatomic site where increase in size or possible treatment-related edema might pose risk to patient (e.g., brain stem lesions). Patients who underwent local treatment for such lesions identified by screening contrast brain MRI might still be eligible for the study based on criteria described under CNS inclusion criteria described above.

(22) Patient might not have known or suspected LMD as documented by the investigator.

(23) Patient might not have poorly controlled (>1/week) generalized or complex partial seizures, or manifest neurologic progression due to brain metastases notwithstanding CNS-directed therapy.

### Criteria for Discontinuation of Study Treatment

Randomized patients were not replaced, including patients who had not received study treatment. Reasons for patient withdrawal from study treatment might be due to any of the following: AE, progressive disease, second disease progression after isolated progression in brain, death, withdrawal of consent, loss to follow-up, physician decision due to clinical progression, physician decision (due to other factors), patient decision, protocol violation, study termination by sponsor, pregnancy or patient begins breast-feeding while on trial, or other criteria as appropriate.

The reason for withdrawal from study treatment must be recorded in the patient's eCRF. Evaluations scheduled for the 30-Day Follow-up Visit and Long-Term Follow-up Visits were completed, unless the patient withdrew consent from the study in writing. Patients also might choose to withdraw consent for procedures and visits but remain on study for PFS and OS follow-up through medical records, public records, or public platform. Patients were also followed for progressive disease (per RECIST 1.1) at least until a PFS event had been observed. If an AE was the cause for withdrawal from study treatment, then “Adverse Event” was recorded as the reason for treatment discontinuation rather than physician decision or patient decision. Treatment discontinuation due to AE was noted any time that a patient had an AE such that the patient might not re-start tucatinib, either due to investigator discretion or due the requirements of dose modification described below (e.g., requiring dose reduction to <150 mg BID tucatinib, holding tucatinib >6 weeks due to toxicity, or lack of resolution of AE to a sufficient grade to re-start tucatinib). Patients who discontinued tucatinib or placebo or both capecitabine and trastuzumab were recorded as an “adverse event” for the reason for treatment discontinuation if AE led to discontinuation of study drugs.

Because the primary study endpoint was defined as PFS as determined by central radiologic assessment, every effort was made to confirm disease progression per RECIST 1.1 whenever possible. However, in instances where patients appeared to have progressive symptoms and signs of metastatic breast cancer for whom it was not possible or feasible to undergo radiologic assessment, investigators might remove the patient from study treatment due to “physician decision due to clinical progression.” These patients were censored in the final analysis of the primary endpoint, so use of this reason for removing such patients from study treatment was restricted to those cases in which it was not clinically appropriate for the patient to undergo further radiologic assessment and where there was clinical confidence for cancer progression in the absence of radiographic confirmation. Special consideration was given to ensure that other possible reasons, particularly AEs, were not a more accurate description of the reason for study drug discontinuation in these cases.

Long-term follow-up after discontinuation of study treatment was continued until patient withdrawal from the study. Patients also might choose to withdraw consent for procedures and visits but remain on study for PFS and OS follow-up through medical records, public records, or public platform. Reasons for patient withdrawal from the study might be due to any of the following: death, withdrawal of consent for follow-up, loss to follow-up, physician decision, study termination by sponsor, or other reason as appropriate.

### Dose Modifications

Tables 2-7 provide dose modification guidance for tucatinib or placebo, capecitabine, and trastuzumab.

All AEs and laboratory abnormalities were assessed by the investigator for relationship to tucatinib or placebo, capecitabine, and trastuzumab, as applicable. An AE might be considered related to tucatinib or placebo alone, capecitabine alone, trastuzumab alone, 2 of the 3 drugs, all 3 drugs, or to none. In the event that the relationship was unclear, discussion was held with the medical monitor to discuss which study drug(s) was held and/or modified. Dosing was modified (including holding the dose, dose reduction, or discontinuation of drug) as described below.

Any study drug was discontinued if a delay of that drug greater than 6 weeks was required due to treatment-related toxicity, unless a longer delay was approved by the medical monitor. For patients who continued capecitabine and/or trastuzumab after discontinuation of tucatinib/placebo in the absence of progression per RECIST 1.1 but who did not initiate additional new anti-cancer therapy, data related to administration of trastuzumab and/or capecitabine continued to be collected. Patients were considered to be no longer receiving study treatment after discontinuation of tucatinib/placebo.

Patients might discontinue either capecitabine or trastuzumab due to toxicity, and continue on tucatinib or placebo in combination with either capecitabine or trastuzumab, as applicable. If both capecitabine and trastuzumab were discontinued, patients also discontinued tucatinib or placebo study treatment and were considered to be no longer receiving study treatment.

Protocol defined visits and cycle numbering continued as planned during a 21-day cycle even during dose holds or delays.

Capecitabine was only taken on Days 1 to 14 of a cycle. No doses were given on Day 15 through Day 21 of a cycle.

Dose reductions or treatment interruption for reasons other than those described below might be made by the investigator if it was deemed in the best interest of patient safety.

Doses held for toxicity were not replaced.

Study treatment might be held up to 6 weeks to allow local CNS therapy. Oral study drugs (tucatinib/placebo and capecitabine) were to be held 1 week prior to planned CNS-directed therapy. The potential for radiosensitization with tucatinib was unknown. Capecitabine was a known radiation sensitizer and therefore needed to be held prior to CNS-directed radiotherapy. Trastuzumab had been shown not to potentiate radiation and therefore might continue as per protocol schedule during radiotherapy. Oral study drugs might be re-initiated 7 days or more after completion of SRS/SRT, 21-days or more after WBRT and 28-days or more after surgical resection. Plans for holding and re-initiating study drugs before and after local therapy required discussion with, and documented approval from, the medical monitor.

### Tucatinib or Placebo Dose Reductions

Tables 2-7 provide the tucatinib or placebo dose modification requirements. Dose reductions larger than those required by these tables might be made at the discretion of the investigator. Up to 3 dose reductions of tucatinib or placebo were allowed, but dose reductions to below 150 mg BID were not allowed. Patients who, in the opinion of the investigator, would require a dose reduction to <150 mg BID, or who would require a potential fourth dose reduction of tucatinib, discontinued study treatment.

Tucatinib or placebo dose was not re-escalated after a dose reduction was made.

TABLE 2
Recommended Tucatinib or Placebo Dose Reduction Schedule

| | 1st Dose | 2nd Dose | 3rd Dose |
|---|---|---|---|
| Starting Dose^{a} | Reduction | Reduction | Reduction |
| 300 mg PO BID | 250 mg PO BID | 200 mg PO BID | 150 mg PO BID |

^{a}Dose reductions of greater steps than those listed in this table (i.e. more than 50 mg per dose reduction) might be made if considered clinically appropriate by the investigator. However, tucatinib or placebo might not be dose-reduced below 150 mg BID.

### Trastuzumab Dose Modifications

There were no dose reductions for trastuzumab. Trastuzumab might also be given on a weekly basis at 2 mg/kg IV q 7 days, but only in the circumstance that trastuzumab infusion had been delayed, and weekly infusions were required to resynchronize the cycle length to 21 days, after discussion with the medical monitor. The subcutaneous dose of trastuzumab (600 mg) cannot be modified as it was administered only once every 3 weeks. If trastuzumab cannot be restarted at the same dose after being held for an AE, it must be discontinued. If dosing of trastuzumab had been held for >4 weeks, the IV loading dose of 8 mg/kg was given per approved dosing instructions or the 600 mg subcutaneous dose should be re-started. As trastuzumab might be given as an IV infusion, infusion-associated reactions (IARs), might occur.

If a significant IAR occurred, the infusion was interrupted and appropriate medical therapies were administered (see below). Permanent discontinuation was considered in patients with severe IAR. This clinical assessment was based on the severity of the preceding reaction and response to administered treatment for the adverse reaction.

If patients developed an IAR, patients were treated according to the following guidelines, or according to institutional guidelines, at discretion of the investigator: stop infusion and notify physician; assess vital signs; administer acetaminophen 650 mg PO; consider administration of meperidine 50 mg IM, diphenhydramine 50 mg IV, ranitidine 50 mg IV or cimetidine 300 mg IV, dexamethasone 10 mg IV, or famotidine 20 mg IV; and if vital signs stable, resume trastuzumab infusion.

No standard premedication was required for future treatments if patients had developed an infusion syndrome. Patients might be given acetaminophen prior to treatments. Serious reactions had been treated with supportive therapy such as oxygen, beta-agonists, corticosteroids and withdrawal of study agent as indicated.

TABLE 3
Dose Modifications of Tucatinib or Placebo and Trastuzumab for Clinical Adverse
Events Other Than Left Ventricular Dysfunction Related to Either Tucatinib or Placebo and/or
Trastuzumab, or Hepatocellular Toxicity*

| | Tucatinib or Placebo | Trastuzumab |
|---|---|---|
| Clinical Adverse Event | Related to tucatinib or | Related to Trastuzumab |
| | Placebo | |
| ≥Grade 3 AEs other than | Hold until severity ≤Grade 1 | Do not administer until |
| Grade 3 fatigue lasting ≤3 | or pretreatment level. | severity |
| days; alopeciaª; nausea; | Restart at next lowest dose | ≤Grade 1 or pretreatment |
| vomiting; diarrhea; rash; | level. | level. |
| correctable electrolyte | | Restart without dose |
| abnormalities which return to | | reduction. |
| ≤Grade 1 within 7 days. | | |
| Grade 3 nausea, vomiting, or | Hold until severity ≤Grade 1 | Do not administer until |
| diarrhea WITHOUT optimal | or pretreatment level. Initiate | severity ≤Grade 1 or |
| use of anti-emetics or anti- | appropriate therapy. | pretreatment level. Initiate |
| diarrheals. | Restart without dose | appropriate therapy. |
| | reduction. | Restart without dose |
| | | reduction. |
| Grade 3 nausea, vomiting, or | or pretreatment level. | Do not administer until |
| diarrhea WITH optimal use of | Hold until severity ≤Grade 1 | severity ≤Grade 1 or |
| anti-emetics or anti- | Restart at next lowest dose | pretreatment level. |
| diarrheals. | level. | Restart without dose |
| | | reduction. |
| Grade 4 nausea, vomiting, or | Do not administer until | Do not administer until |
| diarrhea regardless of use of | severity ≤Grade 1. | severity ≤Grade 1. Restart |
| anti-emetics or anti- | Reduce to next lowest dose | without dose reduction. |
| diarrheals. | level. | |
| Grade 3 rash WITHOUT | Hold until severity ≤Grade 1 | Do not administer until |
| optimal use of topical | or pretreatment level. Initiate | severity ≤Grade 1 or |
| corticosteroids or anti- | appropriate therapy. | pretreatment level. Initiate |
| infectives. | Restart without dose | appropriate therapy. |
| | reduction. | Restart without dose |
| | | reduction. |
| Grade 3 rash WITH optimal | Hold until severity ≤Grade 1 | Do not administer until |
| use of topical corticosteroids | or pretreatment level. | severity ≤Grade 1 or |
| or anti-infectives. | Restart at next lowest dose | pretreatment level. |
| | level. | Restart without dose |
| | | reduction. |
| Grade 4 rash regardless of use | Hold until severity ≤Grade 1 | Do not administer until |
| of topical corticosteroids or | or pretreatment level. | severity ≤Grade 1 or |
| anti-infectives. | Restart at next lowest dose | pretreatment level. |
| | level. | Restart without dose |
| | | reductions. |

^{a}No dose modifications are required for alopecia
*Note that if the AE in question does not recover to the Grade required for restarting study medication as outlined in the table, the patient may need to discontinue the drug completely. Patients requiring a hold of tucatinib for >6 weeks must discontinue study treatment, unless a longer delay is approved by the medical monitor.

### Capecitabine Dose Modifications

Capecitabine doses were modified as described below in Table 4.

Capecitabine was held for any patient who experienced a Grade 2 or greater AE considered related to capecitabine or to the combination of tucatinib or placebo and capecitabine and/or trastuzumab (attribution as determined by the investigator). Held doses of capecitabine were not made up within each cycle.

The capecitabine dose was not re-escalated after a dose reduction was made.

TABLE 4
Dose Modification of Capecitabine for Clinical Adverse Events
Considered Related to Capecitabine

| | | Dose Adjustment for Next |
|---|---|---|
| CTCAE Toxicity | | Treatment (% of Starting |
| Grades | During a Course of Therapy | Dose)ª |
| Grade 1 | Maintain dose level. | Maintain dose level. |

Grade 2^{b}

| | | |
|---|---|---|
| 1^{st} appearance | Interrupt until resolved to | 100% |
| | Grade ≤1. | |
| 2^{nd} appearance | Interrupt until resolved to | 75% |
| | Grade ≤1. | |
| 3^{rd} appearance | Interrupt until resolved to | 50% |
| | Grade ≤1. | |
| 4^{th} appearance | Discontinue permanently. | NA |

Grade 3

| | | |
|---|---|---|
| 1^{st} appearance | Interrupt until resolved to | 75% |
| | Grade ≤1. | |
| 2^{nd} appearance | Interrupt until resolved to | 50% |
| | Grade ≤1. | |
| 3^{rd} appearance | Discontinue permanently. | NA |

Grade 4

| | |
|---|---|
| 1^{st} appearance | Discontinue permanently. |

Abbreviations:
Common Terminology Criteria for Adverse Events (CTCAE); not applicable (NA).
^{a}Dose modification table is based upon XELODA ® package insert; dose rounding is performed per institutional guidelines
^{b}In certain instances of asymptomatic or mildly symptomatic Grade 2 laboratory abnormalities (for example, anemia), investigators may choose to maintain capecitabine dose level and/or to resume capecitabine prior to resolution to Grade 1. This is done only when the risk to patient from capecitabine dose interruption and/or reduction outweighs the risk to the patient from the adverse event, and when the action is consistent with usual and customary clinical practice. If an investigator wishes to follow an alternative dose modification schedule of capecitabine in these circumstances, approval from medical monitor is required.

### Dose Modifications for Hepatotoxicity

Dose modification might be required in the case of liver function abnormalities. For dose modifications of tucatinib or placebo and capecitabine, see Table 5 below. Dose modification of trastuzumab was not required but dosing could be held at investigator discretion. For patients with documented Gilbert's disease, medical monitor was contacted for guidance regarding dose modifications in these patients.

TABLE 5
Dose Modifications of Tucatinib or Placebo and Capecitabine for Liver Function
Abnormalities

| | Action for tucatinib or | |
|---|---|---|
| | placebo, Regardless of | |
| Liver Function Abnormalities | Relationship to Drug | Capecitabine |
| Grade 2 elevation of ALT and/or AST | Dose modification not | If abnormalities |
| (>3-≤5 × ULN) | required | are considered |
| Grade 3 elevation of ALT and/or AST | Hold until severity ≤Grade1 | related to |
| (>5-20 × ULN) | Restart at next lowest dose | capecitabine, |
| | level | modifications are |
| Grade 4 elevation of ALT and/or AST | Discontinue drug | made as per Table |
| (>20 × ULN) | | 4. |
| Elevation of ALT and/or AST (>3 × ULN) | Discontinue drug | If abnormalities |
| AND | | are not considered |
| Bilirubin (>2 × ULN) | | related to |
| Grade 2 elevation of bilirubin (>1.5-3 × | Hold until severity ≤Grade 1 | capecitabine, |
| ULN) | Restart at same dose level | modifications are |
| Grade 3 elevation of bilirubin | Hold until severity ≤Grade 1 | not mandated but |
| (>3-≤10 × ULN) | Restart at next lowest dose | may be made at the |
| | level | discretion of the |
| Grade 4 elevation of bilirubin (> 0 × ULN) | Discontinue drug | investigator. |

Abbreviations:
alanine aminotransferase (ALT); aspartate aminotransferase (AST); upper limit of normal (ULN).

### Dose Modifications for Left Ventricular Dysfunction

Tucatinib or placebo and trastuzumab dose modification guidelines for left ventricular dysfunction are provided in Table 6.

TABLE 6
Dose Modifications for Left Ventricular Dysfunction

| | | LVEF below | | |
|---|---|---|---|---|
| | | institutional limits | | |
| | | of normal and | | |
| | | ≥10% points below | | |
| | | pretreatment | LVEF 40% to | |
| | | baseline, or ≥16% | ≤45% and | |
| | | absolute decrease | decrease is | |
| Symptomatic | | from pretreatment | <10% points | |
| CHF | LVEF <40% | baseline | from baseline | LVEF >45% |
| Discontinue | Do not administer | Do not administer | Continue | Continue |
| tucatinib, | tucatinib, placebo or | tucatinib, placebo or | treatment with | treatment with |
| placebo, and | trastuzumab. | trastuzumab. | tucatinib or | tucatinib or |
| trastuzumab. | Repeat LVEF | Repeat LVEF | placebo and | placebo and |
| | assessment | assessment within 4 | trastuzumab. | trastuzumab. |
| | within 4 weeks. | weeks. | Repeat | |
| | If LVEF | If the LVEF has not | LVEF | |
| | <40% is | recovered to within | assessment | |
| | confirmed, | normal limits and | within 4 weeks. | |
| | discontinue | within15% points | | |
| | tucatinib, | from baseline, | | |
| | placebo, and | discontinue tucatinib, | | |
| | trastuzumab. | placebo, and | | |
| | | trastuzumab, as | | |
| | | applicable. | | |

Abbreviations:
Congestive Heart Failure (CHF); Left Ventricular Ejection Fraction (LVEF).

Permanently discontinued tucatinib or placebo and trastuzumab for persistent (i.e., >4 weeks) LVEF decline or for suspension of dosing on >3 occasions for LVEF decline.

### Dose Modifications for Prolongation of the QTc Interval

Tucatinib or placebo dose modification guidelines for prolongation of the QTc interval are provided in Table 7.

TABLE 7
Dose Modifications of Tucatinib or Placebo for Prolongation of QTc Interval, Regardless
of Relationship to Drug

| | | | | Grade 4 |
|---|---|---|---|---|
| | | | | QTc >501 ms or |
| | | | | >60 ms change from |
| | | | | baseline and |
| | | | | Torsade de pointes |
| | | | | or polymorphic |
| | Grade 1 | | Grade 3 | ventricular |
| | QTc | | QTc >501 ms on | tachycardia or signs |
| | 450-480 | Grade 2 | at least 2 | and symptoms of |
| Occurrence | ms | QTc 481-500 ms | separate ECGs | serious arrhythmia |
| 1^{st} | None | Hold until severity | Hold until | Discontinue |
| occurrence | | ≤Grade 1. Restart | severity ≤Grade | tucatinib/placebo. |
| | | without dose reduction. | 1. Restart at next | |
| | | | lowest dose level. | |
| 2^{nd} | None | Hold until severity | Hold until | NA |
| occurrence | | ≤Grade 1. Restart at next | severity ≤Grade | |
| | | lowest dose level. | 1. Restart at next | |
| | | | lowest dose level. | |
| 3^{rd} | None | Hold until severity | Discontinue | NA |
| occurrence | | ≤Grade 1. Restart at | tucatinib/placebo. | |
| | | next lowest dose level. | | |
| 4^{th} | None | Discontinue | NA | NA |
| occurrence | | tucatinib/placebo. | | |

### Safety Assessments

Safety assessments consisted of monitoring and recording AEs and SAEs; physical examination and vital signs; and measurement of protocol-specified clinical laboratory tests, ECG, and either ECHO or MUGA scans deemed critical to the safety evaluation of the study drug(s). Clinically significant changes in these parameters might be captured as AEs.

The investigator was responsible for the appropriate medical care and the safety of patients who had entered this study. The investigator must document all AEs and notify the sponsor of any SAE experienced by patients who had entered this study.

### Data Monitoring Committee

The independent DMC was responsible for monitoring the safety of patients in the study at regular intervals. The DMC looked at blinded and unblinded data including deaths, discontinuations, dose reductions, AEs, and SAEs on a regular basis. The DMC made recommendations to the sponsor regarding the conduct of the study, including study continuation as planned or with protocol amendment, or early discontinuation of the study for excessive toxicity. A separate DMC Charter outlined the committee's composition, members' roles and responsibilities, and described DMC procedures. The sponsor provided a copy of each DMC recommendation to the investigators.

### Clinical Laboratory Evaluation

All safety labs were analyzed by the site's local laboratory(ies). A central laboratory was used for confirmatory HER2 testing during pre-screening and screening.

The chemistry panel included the following tests: calcium, magnesium, inorganic phosphorus, uric acid, total protein, lactate dehydrogenase (LDH), albumin, blood urea nitrogen (BUN), creatinine, bicarbonate, glucose, potassium, chloride, and sodium.

Liver function tests (LFT) included the following: AST/SGOT, ALT/SGPT, total bilirubin, and alkaline phosphatase.

The hematology panel included the following tests: complete blood count (CBC) with differential, hemoglobin, hematocrit (Hct), and platelets.

The coagulation panel included the following tests: INR, prothrombin time (PT), and aPTT.

The urinalysis included, but is not limited to, the following tests: color, appearance, pH, protein, glucose, ketones, and blood.

### Safety Plan for Cardiotoxicity

Trastuzumab and other HER2-targeted therapies were known to increase the risk of the development of asymptomatic and symptomatic declines in LVEF. There had been rare reports of asymptomatic cardiac failure in patients taking tucatinib in combination with trastuzumab alone or with capecitabine. Cardiac function was therefore monitored closely.

Patients were closely monitored throughout the study for the occurrence of any other expected and/or unexpected toxicities. Assessment of cardiac ejection fraction was performed by MUGA or ECHO at screening and at least once every 12 weeks thereafter until study discontinuation, and 30 days after the last treatment dose (unless done within 12 weeks prior to 30-day follow-up visit).

To correct for heart rate, QT intervals were calculated using the Fridericia formula. The risk of QTc prolongation with tucatinib was not yet fully known. Tucatinib must be administered with caution in patients with conditions which might prolong QTc. These conditions included patients with uncorrected hypokalemia or hypomagnesemia and medications with an accepted or possible association with prolongation of the QTc interval or induction of torsade de pointes (see, Table 13 at the end of this example). Excluded from the study were patients with patients with ≥Grade 2 QTc prolongation on screening ECG, congenital or acquired long QT syndrome, family history of sudden death, a history of previous drug induced QT prolongation and current use of medications with a known and accepted association with QT prolongation (see, Table 13 at the end of this example).

### Safety Plan for Hepatotoxicity

While not among the most common adverse reactions reported in patients taking tucatinib, Grade 3 and 4 elevation of LFTs had been seen in some patients on tucatinib studies. Monitoring of liver function tests was required for any patient taking tucatinib.

Patients had LFTs (ALT, AST, total bilirubin, alkaline phosphatase) monitored closely. Measurement of conjugated and unconjugated bilirubin was considered in cases of hyperbilirubinemia to assist in determination of its etiology. Tucatinib was held according to protocol if liver functions tests were elevated, and monitored for normalization to the appropriate level per protocol before restarting study drugs. Other contributing factors (e.g., concomitant use of hepatotoxic agents) were also considered and modified as clinically appropriate.

The identification of liver enzyme abnormalities as potential adverse reactions to tucatinib did not impact upon the anticipated favorable benefit-risk profile of tucatinib, and was thus far in line with the types and severity of AEs that might be seen with other cancer therapies for patients with metastatic breast cancer.

Safety Plan for Patients with Brain Metastases

Patients with brain metastases were at risk for occurrence of AEs due to the presence of CNS lesions, progression of disease and toxicities potentially related to study treatment. On occasion, treatment of brain metastases with systemic or radiation therapy had been associated with localized edema thought to be due to treatment effect and not tumor progression. A patient in study ONT-380-005 with known brain metastases was found to have cerebral edema in an area surrounding a known metastasis in the thalamus shortly after starting treatment with tucatinib, capecitabine and trastuzumab. The patient's symptoms responded rapidly and completely to systemic corticosteroids. It was not known if this patient's symptoms were due to local progression or treatment-related toxicity. Similarly, a patient treated with tucatinib and trastuzumab alone experienced enlargement of a previously irradiated CNS lesion during study treatment. The patient was taken for surgical resection, and found to have no viable tumor. The resected lesion was thought to represent treatment-related necrosis.

In order to minimize the risk of symptomatic cerebral edema in patients with brain metastases in this study, patients with high-risk metastases, including those requiring immediate local therapy, those with rapidly progressing lesions, those requiring corticosteroids at the start of the study (>2 mg of dexamethasone or equivalent per day) for control of CNS symptoms, and those with larger untreated lesions, were excluded from the trial. However, if these patients were amenable to immediate CNS-directed therapy with either surgery or radiation, they might undergo local therapy and then be eligible for the trial. Under select circumstances patients might receive corticosteroid therapy for acute management of symptomatic local edema, as long as contrast brain MRI did not show clear evidence of CNS progression. All such instances required approval from the study medical monitor.

### Safety Plan for Prevention of Pregnancy

Due to the potential effect on embryo-fetal development, all study patients must practice an effective method of contraception, as described above, starting from the signing of informed consent until 7 months after the last dose of study medication or investigational medicinal product. Women of childbearing potential (i.e., women who had not undergone surgical sterilization with a hysterectomy, bilateral salpingectomy, and/or bilateral oophorectomy; or, were not postmenopausal, as defined as ≥12 months of amenorrhea) must have a negative pregnancy test before beginning the trial and must agree to use a highly effective birth control method. Effective methods of contraception included combined (estrogen and progestogen containing) hormonal contraception associated with inhibition of ovulation (oral, intravaginal, or transdermal); progestogen-only hormonal contraception associated with inhibition of ovulation (oral, injectable, or implantable); intrauterine device; intrauterine hormone-releasing system; bilateral tubal occlusion/ligation; vasectomized partner; or sexual abstinence. Male patients with partners of childbearing potential must use barrier contraception.

Patients of child-bearing potential were to have urine pregnancy tests performed on Day 1 of each treatment cycle.

### Adverse Events

### Definitions

An “adverse event (AE)” is defined as any untoward medical occurrence in a patient or clinical investigation patient administered a pharmaceutical product and which does not necessarily have to have a causal relationship with the treatment methods described herein.

An AE can therefore be any unfavorable and unintended sign (e.g., an abnormal laboratory finding), symptom or disease temporally associated with the use of a medicinal product, whether or not considered related to the medicinal product (International Conference on Harmonisation (ICH) E2A guideline; Definitions and Standards for Expedited Reporting; 21 CFR 312.32 IND Safety Reporting).

The factors below were considered when determining whether or not to record a test result or medical condition as an AE.

Any new undesirable medical occurrence or unfavorable or unintended change of a pre-existing condition that occurred during or after treatment with study drugs was recorded as an AE.

Complications that occurred as a result of protocol-mandated interventions (e.g., invasive procedures such as biopsies) were recorded as an AE.

Elective procedures or routinely scheduled treatment were not considered AEs.

However, an untoward medical event occurring during the pre-scheduled elective procedure was recorded as an AE.

Baseline conditions were not considered AEs unless the condition worsened following study drug administration. Any change assessed as clinically significant worsening of the disease from baseline must be documented as an AE. Baseline conditions present prior to consent were recorded as medical history.

Clinically significant laboratory abnormalities or vital signs (e.g., requiring intervention, meeting serious criteria, resulting in study termination or interruption of study treatment, or associated with signs and symptoms) were recorded as AEs. If possible, abnormal laboratory results that met the definition of an AE were reported as a clinical diagnosis rather than the abnormal value itself(e.g., “anemia” rather than “decreased blood count”).

A “serious adverse event (SAL)” is defined as an AE that meets one of the following criteria:

TABLE 8
Serious Adverse Event Classification

| | |
|---|---|
| Fatal: | The AE resulted in death. |
| Life Threatening: | The AE placed the patient at immediate risk of death. |
| | This classification does not apply to an AE that |
| | hypothetically might cause death if it were more severe. |
| Hospitalization: | The AE required or prolonged an existing inpatient |
| | hospitalization. Hospitalizations for elective medical or |
| | surgical procedures or treatments planned before the |
| | signing of informed consent in the study or routine |
| | check-ups are not SAEs by this criterion. Admission to |
| | a palliative unit or hospice care facility is not considered |
| | to be a hospitalization. Hospitalizations or prolonged |
| | hospitalizations for scheduled therapy of the underlying |
| | cancer or study target disease need not be captured as |
| | SAEs. |
| Disabling/Incapacitating: | Resulted in a substantial and permanent disruption of |
| | the patient's ability to carry out activities of daily living. |
| Congenital Anomaly or | An adverse outcome in a child or fetus of a patient |
| Birth Defect: | exposed to the study drug or study treatment regimen |
| | before conception or during pregnancy. |
| Important medical event: | The AE did not meet any of the above criteria, but could |
| | have jeopardized the patient and might have required |
| | medical or surgical intervention to prevent one of the |
| | outcomes listed above. |

“Overdose” is defined as the administration of a quantity of investigational medicinal product given per administration or cumulatively which is above the maximum dose, according to the protocol.

“Medication error” refers to an unintentional error in dispensing or administration of the investigational medicinal product not in accordance with the protocol described in this example.

“Misuse” is defined as any situation where the investigational medicinal product is intentionally and inappropriately used not in accordance with the protocol.

“Abuse” is defined as the persistent or sporadic intentional excessive use of the investigational medicinal product, which is accompanied by harmful physical or psychological effects.

Information pertaining to overdoses, medication errors, abuse, and misuse was collected as part of investigational medicinal product dosing information and/or as a protocol violation, as required.

Any AE associated with an overdose, medication error, misuse, or abuse of study drug was recorded on the AE eCRF with the diagnosis of the AE.

An “adverse event (AE) of special interest” can be any serious or non-serious AE that is of scientific or medical concern as defined by the sponsor and specific to the program, for which ongoing monitoring and rapid communication to the sponsor may be appropriate.

The following AEs of special interest were reported to the sponsor irrespective of regulatory seriousness criteria or causality within 24 hours.

### Potential Drug-Induced Liver Injury

Any potential case of drug-induced liver injury as assessed by laboratory criteria for Hy's Law was considered as a protocol-defined event of special interest. The following laboratory abnormalities define potential Hy's Law cases: AST or ALT elevations that are >3×ULN with concurrent elevation (within 21 days of AST and/or ALT elevations) of total bilirubin >2×the ULN, except in patients with documented Gilbert's syndrome. Measurement of conjugated and unconjugated bilirubin were considered in cases of hyperbilirubinemia to assist in determination of its etiology.

### Asymptomatic Left Ventricular Systolic Dysfunction

In general, asymptomatic declines in LVEF was reported as AEs since LVEF data were collected separately in the eCRF. However, an asymptomatic decline in LVEF leading to a change in study treatment or discontinuation of study treatment was considered an event of special interest and a serious adverse event, and must be reported to the sponsor.

### Cerebral Edema

Any event of cerebral edema not clearly attributable to progression of disease was reported as an Event of Special Interest.

AE severity was graded using the National Cancer Institute's Common Terminology Criteria for Adverse Events (NCI CTCAE), version 4.03. These criteria are provided in Table 12 at the end of this example.

AE severity and seriousness were assessed independently. Severity characterizes the intensity of an AE. Seriousness is a regulatory definition and serves as a guide to the sponsor for defining regulatory reporting requirements (see definition of SAE above).

The relationship of an AE to all study drugs (tucatinib/placebo, capecitabine, and trastuzumab) was assessed using the guidelines presented in Table 9 below. An AE for which there had been no causal relationship reported required follow-up to determine causality.

TABLE 9
AE Causal Relationship Guidelines

Is the AE/SAE suspected to be caused by the investigational product on the basis of facts,
evidence, science-based rationales, and clinical judgment?

| | |
|---|---|
| Related | The temporal relationship of the AE/SAE to investigational product |
| | administration makes a causal relationship possible AND other drugs, |
| | therapeutic interventions or underlying conditions do not provide sufficient |
| | explanation for the AE/SAE. |
| Not related | The temporal relationship of the AE/SAE to investigational product |
| | administration makes a causal relationship unlikely OR other drugs, |
| | therapeutic interventions, or underlying conditions provide a sufficient |
| | explanation for the AE/SAE. |

### Procedures for Eliciting and Recording Adverse Events

### Eliciting Adverse Events

The investigator assessed patients for the occurrence of AEs at all scheduled and unscheduled visits. The occurrence of AEs was sought by non-direct questioning of the patient at each visit. AEs might also be detected when they were volunteered by the patient during and between visits or through physical examination, or other assessments.

All AEs reported by the patient were reviewed by the investigator and must be recorded on the source documents and AE eCRFs provided.

### Recording Adverse Events

Regardless of relationship to study drug, all serious and non-serious AEs that occurred during the protocol-defined reporting period were to be recorded on the eCRF. SAEs occurring between pre-screening consent and main consent did not need to be documented, unless they were caused by a study procedure (e.g., biopsy).

The following information was assessed and recorded on the eCRF for each AE: description of the AE (including onset and resolution dates), severity (see, definitions above), relationship to each study drug (see, definitions above), outcome of each event, seriousness (see, definitions above), and action taken regarding each study drug.

Diagnosis vs. Signs or Symptoms

Whenever possible, the investigator grouped signs or symptoms that constituted a single diagnosis under a single event term. For example, cough, rhinitis and sneezing might be grouped together as “upper respiratory tract infection.” Grouping of symptoms into a diagnosis was only done if each component sign and/or symptom was a medically confirmed component of a diagnosis as evidenced by standard medical textbooks. If any aspect of a sign or symptom did not fit into a classic pattern of the diagnosis, the individual symptom was reported as a separate event.

### Progression of Underling Malignancy

Since progression of underlying malignancy was being assessed as an efficacy variable, it was not reported as an AE or SAE. Symptomatic clinical deterioration due to disease progression as determined by the investigator also was not reported as an AE or SAE.

However, clinical symptoms of progression might be reported as AEs or SAEs if the symptom could not be determined as exclusively due to progression of the underlying malignancy or did not fit the expected pattern of progression for the disease under study. In addition, complications from progression of the underlying malignancy were reported as AEs or SAEs.

### Reporting Periods and Follow-Up of Adverse Events and Serious Adverse Events

All AEs identified during the clinical study were reported from the time the patient signs informed consent through the 30-day follow-up visit (tucatinib/placebo, capecitabine, or trastuzumab).

Any SAE that occurred after the patient discontinued study treatment considered by the investigator to be related to any study drug was reported to the sponsor.

All SAEs and AEs of special interest was followed until the acute event had resolved or stabilized, even if the patient discontinued study treatment prior to SAE resolution. Non-serious AEs were followed per the reporting period as noted above.

If a non-serious AE was ongoing at the 30-Day Follow-up Visit, the AE was recorded as ongoing.

### Serious Adverse Event and Event of Special Interest Reporting Procedures

All SAEs/EOIs regardless of relationship to a study drug that occurred after the first administration of a study drug must be reported to the sponsor on a SAE/EOI form within 24 hours of discovery of the event. An SAE occurring after informed consent but before administration of study drug and possibly related to a protocol procedure must also be reported to the sponsor within 24 hours of discovery of the event. Any new information or follow-up information pertaining to previously reported SAEs/EOIs was reported to the sponsor within 24 hours of becoming aware of the new or follow-up information.

For initial SAE/EOI reports, available case details were recorded on a SAE/EOI form. At a minimum, the following was included: patient number, AE term(s) (including serious criteria and onset date), study treatment, and causality assessment.

The processes for reporting and documenting SAEs and EOIs were provided in the study binder. Investigators were responsible for reporting these events to their TRB and/or IEC in accordance with federal and local institutional laws and regulations.

New or follow-up information was faxed to the sponsor's clinical safety department. Medical concerns or questions regarding safety were directed to the medical monitor.

The factors below were considered when recording SAEs.

Death was an outcome of an event. The event that resulted in the death were recorded and reported on both an SAE/EOI form and the eCRF.

For hospitalizations, surgical or diagnostic procedures, the illness leading to the surgical or diagnostic procedure were recorded as the SAE, not the procedure itself.

### Sponsor Safety Reporting to Regulatory Authorities

Investigators were required to report all SAEs to the sponsor. The sponsor conducted safety reporting to regulatory authorities, IRBs, and IECs as required per local regulatory reporting requirements. SAEs assessed as related and unexpected (as per IB) to tucatinib/placebo was unblinded by the sponsor to identify study treatment and was reported in accordance with local regulatory reporting requirements. Investigators received all expedited reports in a blinded manner.

### Pregnancy Reporting

Cases of pregnancy were reported through 7 months after the last dose of study drug (tucatinib, capecitabine, or trastuzumab, whichever was latest). If a patient or the female partner of a male patient became pregnant during participation in the study, the sponsor must be notified. If a study participant became pregnant during administration of the drug, treatment was discontinued.

The investigator reported all pregnancies within 24 hours to the sponsor including the partners of male patients. The sponsor asked for follow up evaluation of the pregnancy, fetus, and child.

Abortion, whether accidental, therapeutic, or spontaneous, was reported as a SAE. Congenital anomaly or birth defects was also reported as a SAE as described above. All pregnancies were monitored for the full duration; all perinatal and neonatal outcomes were reported. Infants were followed for a minimum of 8 weeks. Pregnancy was reported to the sponsor's clinical safety department on a Pregnancy Report Form.

TABLE 10
Selected Strong Inhibitors and Inducer of CYP2C8 and
Their Elimination Half-Lives

| | Elimination Half-life |
|---|---|
| Drug^{a,b} | (hours) |
| Strong Inhibitors | |
| Clopidogrel | 6 hours |
| Gemfibrozil | 1-2 hours |
| Montelukast | 3-6 hours |
| Trimethoprim | 8-12 hours |
| Strong Inducer | |
| Rifampin | 3-5 hours |

^{a}FDA. Drug Development and Drug Interactions: Table of Substrates, Inhibitors and Inducers” (www.fda.gov/Drugs/DevelopmentApprovalProcess/DevelopmentResources/DrugInteractionsLabeling/ucm093664.htm#potency).
^{b}EMA. “Guideline on the investigation of drug interactions” www.ema.europa.eu/docs/en_GB/document_library/Scientific_guideline/2012/07/WC500129606.pdf
c. Drug package insert

TABLE 11
Selected Strong Inhibitors or Inducers of CYP3A4 and Their
Elimination Half-Lives

| | Elimination Half-life^{d} |
|---|---|
| Drug^{a,b,c} | (hours) |
| Strong Inhibitors | |
| Macrolide Antibiotics | |
| Clarithromycin, | 3-7 hours |
| Troleanomycin | 2 hours |
| Azole Antifungals | |
| Itraconazole | 16-28 hours (single dose), |
| | 34-42 hours (repeat dose) |
| Ketoconazole (systemic) | 2-8 hours |
| Voriconazole | Dose dependent |
| Posoconazole | 27-35 hours |
| Other | |
| Nefazodone | 2-4 hours |
| Diltiazem | 3-4 hours |
| White grapefruit juice | ~4-5 hours^{e} |
| Strong Inducers | |
| Barbiturates | Variable |
| Carbamazepine | 25-65 hours (single does), |
| | 12-17 hours (repeat dose) |
| Phenytoin | 7-42 hours |
| Rifampin | 3-4 hours (single does), |
| | 2-3 hours (repeat dose) |
| St. John's Wort | 9-43 hours^{f} |

^{a}FDA. “Drug Development and Drug Interactions: Table of Substrates, Inhibitors and Inducers” (http://www.fda.gov/Drugs/DevelopmentApprovalProcess/DevelopmentResources/DrugInteractionsLabeling/ucm093664.htm#potency).
^{b}EMA. “Guideline on the investigation of drug interactions” www.ema.europa.eu/docs/en_GB/document_library/Scientific_guideline/2012/07/WC500129606.pdf
^{c}Strong CYP3A inhibitors are defined as those drugs that increase the AUC of oral midazolam or other CYP3A substrates ≥5-fold. Ritonavir, indinavir, nelfinavir, atazanivir, and saquinavir are also strong CYP3A3 inhibitors, but would not be used in this study as patients with known HIV are excluded.
^{d}Drug package insert
^{e}Bailey et al., Br J Clin Pharmacol 1998; 46: 101-110
^{f}Kerb et al., Antimicrob Agent & Chemother 1996, 40(9): 2087-2093

TABLE 12
Adverse Event Severity Grading Scale (CTCAE Version 4.03)

| Severity | Grade | Description |
|---|---|---|
| Mild | 1 | Asymptomatic or mild symptoms; clinical or diagnostic |
| | | observations only; intervention not indicated |
| Moderate | 2 | Minimal, local or noninvasive intervention indicated; |
| | | limiting age-appropriate instrumental activities of daily |
| | | living (ADL). Instrumental ADL refer to preparing |
| | | meals, shopping for groceries or clothes, using the |
| | | telephone, managing money, etc. |
| Severe | 3 | Medically significant but not immediately life- |
| | | threatening; hospitalization or prolongation of |
| | | hospitalization indicated; disabling; limiting self-care |
| | | ADL. Self-care ADL refer to bathing, dressing and |
| | | undressing, feeding self, using the toilet, taking |
| | | medications, and not bedridden. |
| Life- | 4 | Life-threatening consequences; urgent intervention |
| threatening | | indicated |
| Death | 5 | Death related to adverse event. |

TABLE 13
Drugs Accepted or Possibly Associated with Risk of QT Prolongation
or Torsade de Pointes

| | Anti- | Anti- | Opioid | Anti- |
|---|---|---|---|---|
| | infectives | psychotics | analgesics | histamines |
| Accepted | Clarithromycin | Haloperidol | Methadone | Terfaenadine |
| association | Erythromycin | Chlorpromazine | | |
| | Chloroquine | | | |
| | Pentamidine | | | |
| Possibly | Azithromycin | Resperidone | | |
| associated | Roxithromycin | Quetiapine | | |
| | Telithromycin | Sertinodole | | |
| | Moxifloxacin | Zisprasidone | | |
| | Amantadine | Lithium | | |
| | | Clozapine | | |
| | | Anti-emetics/ | | |
| | Anti- | Gastric | | Anti- |
| | depressants | motility drugs | Anti-cancer | arrythmics |
| Accepted | | Domperidone | | Amiodarone |
| association | | Cisapride | | Sotalol |
| | | | | Disopyramide |
| | | | | Dofetilide |
| | | | | Procainamide |
| | | | | Quinidine |
| Possibly | Escitalopram | Ondansetron | Tamoxifen | |
| associated | Venlaxafine | Dolasteron | Nilotinib | |
| | | Granisetron | Lapatinib | |

Guidance for Industry, E14 Clinical Evaluation of QT/QTc Interval Prolongation and Proarrhythmic Potential for Non-Antiarrhythmic Drugs. U.S. Department of Health and Human Services, Food and Drug Administration, Center for Drug Evaluation and Research (CDER), Center for Biologics Evaluation and Research (CBER) October 2005, ICH.

Geoffrey K Isbister and Colin B Page. Drug induced QT prolongation: the measurement and assessment of the QT interval in clinical practice. Br J Clin Pharmacol. 2013 July; 76(1): 48-57.

### Results

The trial met the primary endpoint of progression-free survival (PFS), showing tucatinib in combination with trastuzumab and capecitabine was superior to trastuzumab and capecitabine alone, with a 46 percent reduction in the risk of disease progression or death (hazard ratio (HR)=0.54 (95% CI: 0.42, 0.71); p<0.00001). The trial also met the two key secondary endpoints at interim analysis. Tucatinib in combination with trastuzumab and capecitabine demonstrated an improvement in overall survival, with a 34 percent reduction in the risk of death (HR=0.66 (95% CI: 0.50, 0.88); p=0.0048) compared to trastuzumab and capecitabine alone. For patients with brain metastases, tucatinib in combination with trastuzumab and capecitabine also demonstrated superior PFS, with a 52 percent reduction in the risk of disease progression or death compared to those who received trastuzumab and capecitabine alone (HR=0.48 (95% CI: 0.34, 0.69); p<0.00001).

Tucatinib in combination with trastuzumab and capecitabine was generally well tolerated with a manageable safety profile. The most frequent adverse events in the group administered tucatinib in combination with trastuzumab and capecitabine included diarrhea, palmar-plantar erythrodysaesthesia syndrome (PPE), nausea, fatigue, and vomiting. Grade 3 or greater adverse events in the group administered tucatinib in combination with trastuzumab and capecitabine compared to the group administered trastuzumab and capecitabine alone included diarrhea (12.9 vs. 8.6 percent), increased aspartate aminotransferase (AST) (4.5 vs. 0.5 percent), increased alanine aminotransferase (ALT) (5.4 vs. 0.5 percent) and increased bilirubin (0.7 vs. 2.5 percent). Importantly, there was no requirement for prophylactic antidiarrheals. Adverse events leading to discontinuations were infrequent in both the group administered tucatinib in combination with trastuzumab and capecitabine and the group administered trastuzumab and capecitabine (5.7 and 3.0 percent).

A brief overview of the study is provided in Table 14.

TABLE 14
Summary of Disposition

| | Tuc + Cap + Tra | Pbo + Cap + Tra | Total |
|---|---|---|---|
| | (N = 320) | (N = 160) | (N = 480) |
| | n (%) | n (%) | n (%) |
| Subjects randomized | 320 (100) | 160 (100) | 480 (100) |
| Subjects who received at least one dose of | 317 (99.1) | 157 (98.1) | 474 (98.8) |
| tucalinib/placebo | | | |
| Subjects on lucatinib/placebo | 66 (20.6) | 11 (6.9) | 77 (16.0) |
| Subjects off tucatinib/placebo | 251 (78.4) | 146 (91.3) | 397 (82.7) |
| Subjects who never received tucatinib/placebo | 3 (0.9) | 3 (1.9) | 6 (1.3) |
| Subjects in long term follow up | 122 (38.1) | 64 (40.0) | 186 (38.8) |
| Subjects of study | 132 (41.3) | 85 (53.1) | 217 (45.2) |
| Reason for study discontinuation | | | |
| Death | 116 (36.3) | 80 (50.0) | 196 (40.8) |
| Withdrawal of consent | 14 (4.4) | 4 (2.5) | 18 (3.8) |
| Lost to follow-up | 2 (0.6) | 0 | 2 (0.4) |
| Physician decision | 0 | 1 (0.6) | 1 (0.2) |

The study was well-balanced across both arms as is shown in Table 15.

TABLE 15
Summary of Demographics and Baseline Subject Characteristics

| | Tuc + Cap + Tra | Pbo + Cap + Tra | Total |
|---|---|---|---|
| | (N = 320) | (N = 160) | (N = 480) |
| Age category, n (%) | | | |
| <65 years | 252 (78.8) | 132 (82.5) | 384 (80.0) |
| >=65 years | 68 (21.3) | 28 (17.5) | 96 (20.0) |
| Gender, n (%) | | | |
| Male | 3 (0.9) | 2 (1.3) | 5 (1.0) |
| Female | 317 (99.1) | 158 (98.8) | 475 (99.0) |
| ECOG performance status, n (%) | | | |
| 0 | 159 (49.7) | 76 (47.5) | 235 (49.0) |
| 1 | 161 (50.3) | 84 (52.5) | 245 (51.0) |
| Estrogen/progesterone receptor status, n (%) | | | |
| Positive for either or both | 190 (59.4) | 99 (61.9) | 289 (60.2) |
| Negative for both | 126 (39.4) | 61 (38.1) | 187 (39.0) |
| Other | 4 (1.3) | 0 | 4 (0.8) |
| Subjects with history of brain metastases or | 148 (46.3) | 71 (44.4) | 219 (45.6) |
| brain metastases at study entry, n (%) | | | |
| Median prior lines of systemic therapy | 4.0 | 4.0 | 4.0 |
| Measurable disease by BIGR, n (%) | | | |
| Yes | 273 (85.3) | 137 (85.6) | 410 (85.4) |
| No | 47 (14.7) | 23 (14.4) | 70 (14.6) |

Patients administered the combination of tucatinib, capecitabine, and trastuzumab exhibited superior progression-free survival with a 46% reduction in the risk of progression or death (see Table 16).

TABLE 16
Progression-Free Survival

| | Tuc + Cap + Tra | Pbo + Cap + Tra |
|---|---|---|
| | (N = 320) | (N = 160) |
| Subjects with progression or death , n (%) | 178 (55.6) | 97 (60.6) |
| Stratified Hazard Ratio (95% C.I.) | 0.544 (0.420, 0.705) | |
| Stratified Log-rank p-value | <00001 | |
| Median PFS (Months) (95% C.I.) | 7.8 (7.5, 9.6) | 5.6 (4.2, 7.1) |
| 25^{th}, 75^{th} percentile | 4.3, 17.8 | 3.0, 9.7 |
| Observed min, max | 0.0+, 34.6+ | 0.0+, 24.0+ |
| Estimated progression free rate at | | |
| 9 months (95% C.I.) | 46.8% (40.5%, 52.9%) | 31.6% (22.9%, 40.7%) |
| 12 months (95% C.I.) | 33.1% (26.6%, 39.7%) | 12.3% (6.0%, 20.9%) |
| 15 months (95% C.I.) | 27.0% (20.5%, 33.8%) | 12.3% (6.0%, 20.9%) |

Patients administered the combination of tucatinib, capecitabine, and trastuzumab also exhibited superior overall survival with a 34% reduction in the risk of death (see Table 17).

TABLE 17
Overall Survival

| | Tuc + Cap + Tra | Pbo + Cap + Tra |
|---|---|---|
| | (N = 410) | (N = 202) |
| Subjects with progression or death, n (%) | 130 (31.7) | 85 (42.1) |
| Stratified Hazard Ratio (95% C.I.) | 0.662 (0.501. 0.875) | |
| Stratified Log-rank p-value | 0.00480 | |
| Median PFS (Months) (95% C.I.) | 21.9 (18.3, 31.0) | 17.4 (13.6, 19.9) |
| 25^{th}, 75^{th} percentile | 12.2, — | 10.2, — |
| Observed min, max | 0.1+, 35.9+ | 0.1+, 33.9+ |
| Estimated progression-free rate at | | |
| 24 months (95% C.I.) | 44.9% (36.6%, 52.8%) | 26.6% (15.7%, 38.7%) |
| 30 months (95% C.I.) | 42.8% (34.0%, 51.3%) | 26.6% (15.7%, 38.7%) |

In patients having brain metastasis, those administered the combination of tucatinib, capecitabine, and trastuzumab exhibited superior progression-free survival with a 52% reduction in the risk of progression or death (see Table 18).

TABLE 18
Progression-Free Survival in Patients having Brain Metastasis

| | Tuc + Cap + Tra | Pbo + Cap + Tra |
|---|---|---|
| | (N = 198) | (N = 93) |
| Subjects with progression or death, n (%) | 106 (53.5) | 51 (54.8) |
| Stratified Hazard Ratio (95% C.I.) | 0.483 (0.339, 0.689) | |
| Stratified Log-rank p-value | <00001 | |
| Median PFS (Months) (95% C.I.) | 7.6 (6.2, 9.5) | 5.4 (4.1. 5.7) |
| 25^{th}, 75^{th} percentile | 4.2, 11.8 | 3.0, 7.5 |
| Observed min, max | 0.0+, 34.6+ | 0.0+, 11.6 |
| Estimated progression-free rate at | | |
| 9 months (95% C.I.) | 43.4% (34.9%, 51.5%) | 14.8% (5.5%, 28.4%) |
| 12 months (95% C.I.) | 24.9% (16.5%, 34.3%) | —* |

The benefits observed in this study were consistent across subgroups, including those based on age, race, hormone receptor status (positive or not positive), presence or absence of baseline brain metastasis, Eastern Cooperative Oncology Group (ECOG) performance status (0 or 1), or location (e.g., North America or Rest of World).

The objective response per BICR in subjects with measurable disease is summarized in Table 19.

TABLE 19

| | Tuc + Cap + Tra | Pbo + Cap + Tra |
|---|---|---|
| | (N = 340) | (N = 171) |
| Best Overall Response , n (%) | | |
| Complete Response (CR) | 3 (0.9) | 2 (1.2) |
| Partial Response (PR) | 135 (39.7) | 37 (21.6) |
| Stable Disease (SD) | 155 (45.6) | 100 (58.5) |
| Progressive Disease (PD) | 27 (7.9) | 24 (14.0) |
| Not Evaluable (NE) | 0 | 1 (0.6) |
| Not Available | 20 (5.9) | 7 (4.1) |
| Subjects with Objective Response | 138 | 39 |
| of commmmed CR or PR, n | | |
| Objective response rate (ORR) % | 40.6 | 22.8 |
| 95% CI for ORR | (35.3, 46.0) | (16.7, 29.8) |

| | |
|---|---|
| Stratified CMH p-value for ORR | 0.00008 |

Over the course of the clinical trial, greater exposure was observed in the group administered tucatinib in combination with trastuzumab and capecitabine (see Table 20).

TABLE 20
Summary of Tucatinib/Placebo Administration

| | Tuc + Cap + Tra | Pbo + Cap + Tra |
|---|---|---|
| | (N = 317) | (N = 157) |
| Number of subjects receiving at | 317 (100) | 157 (100) |
| least one dose of tucatinib/placebo | | |
| Duration of tucatinib/placebo | | |
| exposure (months) | | |
| Mean (STD) | 8.4 (6.9) | 5.9 (4.6) |
| Median | 7.3 | 4.4 |
| Min, Max | <0.1. 35.1 | <0.1, 24.0 |
| Number of treatment cycles initiated | | |
| Mean (STD) | 12.0 (9.7) | 8.4 (6.5) |
| Median | 10.0 | 6.0 |
| Min, Max | 1, 51 | 1, 35 |

From a safety standpoint, the combination of tucatinib, capecitabine, and trastuzumab was well-tolerated with a manageable safety profile and a low rate of discontinuation due to adverse events. Elevated liver function tests and increased diarrhea were manageable with supportive case and dose modification. No other adverse event of special interest (AESI) or unexpected safety findings were observed. No deaths associated with liver injury occurred. Table 21 provides a summary of the most frequent all grate treatment emergent adverse events by preferred term.

TABLE 21

| | Tuc + Cap + Tra | Pbo + Cap + Tra |
|---|---|---|
| | (N = 404) | (N = 197) |
| Preferred Term | n (%) | n (%) |
| Subjects with any event | 401 (99.3) | 191 (97.0) |
| Diarrhoea | 327 (80.9) | 105 (53.3) |
| Palmar-plantar | 256 (63.4) | 104 (52.0) |
| erythrodysaesthesia syndrome | | |
| Nausea | 236 (58.4) | 86 (43.7) |
| Fatigue | 182 (45.0) | 85 (43.1) |
| Vomiting | 145 (35.9) | 50 (25.4) |
| Stomatitis | 103 (25.5) | 28 (14.2) |
| Decreased appetite | 100 (24.8) | 39 (19.8) |
| Headache | 87 (21.5) | 40 (20.3) |
| Aspartale aminotransferase | 86 (21.3) | 22 (11.2) |
| increased | | |
| Alanine aminotransferase | 81 (20.0) | 13 (6.6) |
| increased | | |
| Anaemia | 80 (19.8) | 23 (11.7) |
| Blood bilirubin increased | 75 (18.6) | 20 (10.2) |
| Hypokalemia | 64 (15.8) | 24 (12.2) |
| Abdominal pain | 59 (14.6) | 31 (15.7) |
| Arthralgia | 59 (14.6) | 9 (4.6) |
| Constipation | 59 (14.6) | 39 (19.8) |
| Cough | 57 (14.1) | 23 (11.7) |
| Blood creatinine increased | 56 (13.9) | 3 (1.5) |

Table 22 provides a summary of the most frequent grade 3 or higher treatment emergent adverse events by preferred term.

TABLE 22

| | Tuc + Cap + Tra | Pbo + Cap + Tra |
|---|---|---|
| | (N = 404) | (N = 197) |
| Preferred Term | n (%) | n (%) |
| Subjects with any event | 223 (55.2) | 96 (48.7) |
| Palmar-plantar | 53 (13.1) | 18 (9.1) |
| erythrodysaesthesia syndrome | | |
| Diarrhoea | 52 (12.9) | 17 (8.6) |
| Alanine aminotransferase | 22 (5.4) | 1 (0.5) |
| increased | | |
| Fatigue | 19 (4.7) | 8 (4.1) |
| Aspartale aminotransferase | 18 (4.5) | 1 (0.5) |
| increased | | |
| Anaemia | 15 (3.7) | 5 (2.5) |
| Nausea | 15 (3.7) | 6 (3.0) |

A summary of deaths observed during the study is provided in Table 23. As shown, the majority of deaths were due to disease progression.

TABLE 23

| | Tuc + Cap + Tra | Pbo + Cap + Tra | Total |
|---|---|---|---|
| | (N = 410) | (N = 202) | (N = 612) |
| | n (%) | n (%) | n (%) |
| Total number of deaths | 130 (31.7) | 85 (42.1) | 215 (35.1) |
| Primary cause of death^{a} | | | |
| Disease progression | 109 (83.8) | 71 (83.5) | 180 (83.7) |
| Unknown | 14 (10.6) | 7 (8.2) | 21 (9.8) |
| Other | 1 (0.8) | 2 (2.4) | 3 (1.4) |
| AE | 6 (4.6) | 5 (5.9) | 11 (5.1) |

### Glossary and Terms

| | |
|---|---|
| 5FU | 5-fluorouracil |
| ADL | activities of daily living |
| AE | adverse event |
| ALT/SGPT | alanine aminotransferase/serum glutamic-pyruvate transaminase |
| ANC | absolute neutrophil count |
| anti-HBc | antibodies to Hepatitis B core |
| anti-HCV | antibodies to Hepatitis C virus |
| API | active pharmaceutical ingredient |
| aPTT | activated partial thromboplastin time |
| AR | adverse reaction |
| AST/SGOT | aspartate aminotransferase/serum glutamic-oxaloacetic transaminase |
| AUC | area under the curve |
| BICR | blinded independent central review |
| BID | twice daily |
| BUN | blood urea nitrogen |
| CBC | complete blood count |
| CBR | clinical benefit rate |
| CHF | congestive heart failure |
| CI | confidence interval |
| Cₘₐₓ | maximum concentration observed |
| CNS | central nervous system |
| CR | complete response |
| CT | computed tomography |
| CTCAE | Common Toxicity Criteria for Adverse Events |
| ctDNA | circulating tumor DNA |
| DCC | Data Coordinating Center |
| DDI | drug-drug interaction |
| DFS | disease-free survival |
| DMC | Data Monitoring Committee |
| DNA | deoxyribonucleic acid |
| DOR | Duration of Response |
| ECG | electrocardiogram |
| ECHO | echocardiogram |
| ECOG PS | Eastern Cooperative Oncology Group Performance Status |
| eCRF | electronic case report form |
| ED | emergency department |
| EGFR | epidermal growth factor receptor |
| EOI | event of interest |
| EU | European Union |
| FDA | Food and Drug Administration |
| FISH | fluorescence in situ hybridization |
| GCP | Good Clinical Practice |
| GI | gastrointestinal |
| HBsAg | hepatitis B surface antigen |
| HC | Health Canada |
| Hct | hematocrit |
| HER1 | human epidermal growth factor receptor 1 |
| HER2 | human epidermal growth factor receptor 2 |
| HER2+ | human epidermal growth factor receptor 2 positive |
| HIV | human immunodeficiency virus |
| HR | hazard ratio |
| IAR | infusion-associated reaction |
| IB | Investigator's Brochure |
| ICF | Informed Consent Form |
| ICH | International Conference on Harmonisation |
| IHC | immunohistochemistry |
| ILD | interstitial lung disease |
| INR | international normalized ratio |
| IUD | intrauterine device |
| IV | intravenous |
| IRB/IEC | Institutional Review Board/Independent Ethics Committee |
| IRT | Interactive Response Technology |
| ITT | Intent-to-Treat |
| kg | kilogram |
| LDH | lactate dehydrogenase |
| LFT | liver function test |
| LMD | leptomeningeal disease |
| LVEF | left ventricular ejection fraction |
| MedDRA | Medical Dictionary for Regulatory Activities |
| mg | milligram |
| mL | milliliter |
| mm | millimeter |
| MRI | magnetic resonance imaging |
| mRNA | messenger ribonucleic acid |
| MTD | maximum-tolerated dose |
| MUGA | multiple-gated acquisition scan |
| NCI | National Cancer Institute |
| ONT-380 | Investigational small molecule inhibitor of HER2 |
| (tucatinib) | |
| ORR | objective response rate |
| OS | overall survival |
| PD | progressive disease |
| PET | positron emission tomography |
| PFS | progression-free survival |
| P-gp | P-glycoprotein |
| PIC | powder in capsule |
| PK | pharmacokinetics |
| PO | oral administration |
| PPE | palmar-plantar erythrodysaesthesia |
| PR | partial response |
| PT | prothrombin time |
| PVP-VA | polyvinylpyrrolidine-vinyl acetate copolymer |
| QTc | corrected QT |
| RANO-BM | Response Assessment in Neuro-Oncology - Brain Metastases |
| RD | recommended dose |
| RECIST | Response Evaluation Criteria In Solid Tumors |
| RNA | ribonucleic acid |
| RP2D | recommended Phase 2 dose |
| SAE | serious adverse event |
| SAP | statistical analysis plan |
| SD | stable disease |
| SOC | system organ class |
| SRS | stereotactic radiosurgery |
| SUSAR | suspected unexpected serious adverse reaction |
| T-DM1 | ado-trastuzumab emtansine or trastuzumab emtansine |
| TEAE | treatment-emergent adverse event |
| TKI | tyrosine kinase inhibitor |
| Tucatinib | Investigational small molecule inhibitor of HER2 |
| (ONT-380) | |
| UGT1A1 | UDP-glucuronosyltransferase 1A1 |
| ULN | upper limit of normal |
| WBRT | whole brain radiation therapy |

### REFERENCES

1. Cancer Incidence and Mortality Worldwide: IARC CancerBase No. 10 [Internet]. International Agency for Research on Cancer. 2010 [cited Jun. 6, 2013]. Available from: globocan.iarc.fr.
2. Group USCSW. United States Cancer Statistics: 1999-2009 Incidence and Mortality Web-based Report. In: Health and Human Services CfDCaP, and National Cancer Institute, editor. Atlanta, GA2013.
3. Owens M A, Horten B C, Da Silva M M. HER2 amplification ratios by fluorescence in situ hybridization and correlation with immunohistochemistry in a cohort of 6556 breast cancer tissues. Clinical breast cancer. 2004; 5(1):63-9.
4. Giordano S H, Temin S, Kirshner J J, Chandarlapaty S, Crews J R, Davidson N E, et al. Systemic therapy for patients with advanced human epidermal growth factor receptor 2-positive breast cancer: American Society of Clinical Oncology clinical practice guideline. Journal of clinical oncology: official journal of the American Society of Clinical Oncology. 2014; 32(19):2078-99.
5. Slamon D J, Clark G M, Wong S G, Levin W J, Ullrich A, McGuire W L. Human breast cancer: correlation of relapse and survival with amplification of the HER-2/neu oncogene. Science. 1987; 235(4785):177-82.
6. Slamon D J, Leyland-Jones B, Shak S, Fuchs H, Paton V, Bajamonde A, et al. Use of chemotherapy plus a monoclonal antibody against HER2 for metastatic breast cancer that overexpresses HER2. The New England journal of medicine. 2001; 344(11):783-92.
7. Verma S, Miles D, Gianni L, Krop I E, Welslau M, Baselga J, et al. Trastuzumab emtansine for HER2-positive advanced breast cancer. The New England journal of medicine. 2012; 367(19):1783-91.
8. Baselga J, Cortes J, Kim S B, Im S A, Hegg R, Im Y H, et al. Pertuzumab plus trastuzumab plus docetaxel for metastatic breast cancer. The New England journal of medicine. 2012; 366(2):109-19.
9. Geyer C E, Forster J, Lindquist D, Chan S, Romieu C G, Pienkowski T, et al. Lapatinib plus capecitabine for HER2-positive advanced breast cancer. The New England journal of medicine. 2006; 355(26):2733-43.
10. (lapatinib) T. [package insert]: Novartis; 2015 [cited 2015]. Available from: www.gsksource.com/pharma/content/dam/GlaxoSmithKline/US/en/Prescribing_Information/Tykerb/pdf/TYKERB-PI-PIL.PDF.
11. Vogel C L, Cobleigh M A, Tripathy D, Gutheil J C, Harris L N, Fehrenbacher L, et al. Efficacy and safety of trastuzumab as a single agent in first-line treatment of HER2-overexpressing metastatic breast cancer. Journal of clinical oncology: official journal of the American Society of Clinical Oncology. 2002; 20(3):719-26.
12. Lu Y, Zi X, Zhao Y, Mascarenhas D, Pollak M. Insulin-like growth factor-I receptor signaling and resistance to trastuzumab (Herceptin). Journal of the National Cancer Institute. 2001; 93(24):1852-7.
13. Scaltriti M, Rojo F, Ocana A, Anido J, Guzman M, Cortes J, et al. Expression of p95HER2, a truncated form of the HER2 receptor, and response to anti-HER2 therapies in breast cancer. Journal of the National Cancer Institute. 2007; 99(8):628-38.
14. Pohlmann P R, Mayer I A, Mernaugh R. Resistance to Trastuzumab in Breast Cancer. Clinical cancer research: an official journal of the American Association for Cancer Research. 2009; 15(24):7479-91.
15. Nahta R, Esteva F J. HER2 therapy: molecular mechanisms of trastuzumab resistance. Breast cancer research: BCR. 2006; 8(6):215.
16. Baselga J, Bradbury I, Eidtmann H, Di Cosimo S, de Azambuja E, Aura C, et al. Lapatinib with trastuzumab for HER2-positive early breast cancer (NeoALTTO): a randomised, open-label, multicentre, phase 3 trial. Lancet. 2012; 379(9816):633-40.
17. Blackwell K L, Burstein H J, Storniolo A M, Rugo H, Sledge G, Koehler M, et al. Randomized study of Lapatinib alone or in combination with trastuzumab in women with ErbB2-positive, trastuzumab-refractory metastatic breast cancer. Journal of clinical oncology: official journal of the American Society of Clinical Oncology. 2010; 28(7):1124-30.
18. Blackwell K L, Burstein H J, Storniolo A M, Rugo H S, Sledge G, Aktan G, et al. Overall survival benefit with lapatinib in combination with trastuzumab for patients with human epidermal growth factor receptor 2-positive metastatic breast cancer: final results from the EGF104900 Study. Journal of clinical oncology: official journal of the American Society of Clinical Oncology. 2012; 30(21):2585-92.
19. Network NCC. NCCN Guidelines®& Clinical Resources 2013 [cited 2013 Aug. 15]. Available from: www.nccn.org.
20. Clayton A J, Danson S, Jolly S, Ryder W D, Burt P A, Stewart A L, et al. Incidence of cerebral metastases in patients treated with trastuzumab for metastatic breast cancer. British journal of cancer. 2004; 91(4):639-43.
21. Goldhirsch A, Gelber R D, Piccart-Gebhart M J, de Azambuja E, Procter M, Suter T M, et al. 2 years versus 1 year of adjuvant trastuzumab for HER2-positive breast cancer (HERA): an open-label, randomised controlled trial. Lancet. 2013; 382(9897):1021-8.
22. Pestalozzi B C, Holmes E, de Azambuja E, Metzger-Filho 0, Hogge L, Scullion M, et al. CNS relapses in patients with HER2-positive early breast cancer who have and have not received adjuvant trastuzumab: a retrospective substudy of the HERA trial (BIG 1-01). The Lancet Oncology. 2013; 14(3):244-8.
23. Lin N U, Bellon J R, Winer E P. CNS metastases in breast cancer. Journal of clinical oncology: official journal of the American Society of Clinical Oncology. 2004; 22(17):3608-17.
24. Ekenel M, Hormigo A M, Peak S, Deangelis L M, Abrey L E. Capecitabine therapy of central nervous system metastases from breast cancer. Journal of neuro-oncology. 2007; 85(2):223-7.
25. Ramakrishna N, Temin S, Chandarlapaty S, Crews J R, Davidson N E, Esteva F J, et al. Recommendations on disease management for patients with advanced human epidermal growth factor receptor 2-positive breast cancer and brain metastases: American Society of Clinical Oncology clinical practice guideline. Journal of clinical oncology: official journal of the American Society of Clinical Oncology. 2014; 32(19):2100-8.

1. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy, the subject exhibits progression-free survival of at least 7.5 months following administration of the combination therapy.

2. The method of claim 1, wherein the subject exhibits progression-free survival of at least eight months following administration of the combination therapy.

3. The method of any one of claims 1 or 2, wherein the subject exhibits progression-free survival of at least nine months following administration of the combination therapy.

4. The method of any one of claims 1-3, wherein the subject exhibits progression-free survival of at least ten months following administration of the combination therapy.

5. The method of any one of claims 1-4, wherein the subject has a brain metastasis.

6. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy, the subject exhibits an overall survival of at least eighteen months following administration of the combination therapy.

7. The method of claim 6, wherein the subject exhibits an overall survival of at least nineteen months following administration of the combination therapy.

8. The method of any one of claims 6 or 7, wherein the subject exhibits an overall survival of at least twenty-two months following administration of the combination therapy.

9. The method of any one of claims 6-8, wherein the subject exhibits an overall survival of at least twenty-six months following administration of the combination therapy.

10. The method of any one of claims 6-9, wherein the subject exhibits an overall survival of at least thirty months following administration of the combination therapy.

11. The method of any one of claims 6-10, wherein the subject has a brain metastasis.

12. A method of treating or ameliorating brain metastasis in a subject having HER2 positive breast cancer, the method comprising administering to the subject an effective amount of a combination therapy comprising tucatinib, capecitabine, and trastuzumab.

13. The method of claim 12, wherein the time to additional intervention for treatment of the brain metastasis in the subject has been increased.

14. The method of any one of claims 12 or 13, wherein the need for additional intervention for treatment of the brain metastasis in the subject has been prevented.

15. The method of claim 14, wherein the additional intervention is selected from the group consisting of radiation, surgery, and a combination thereof.

16. The method of any one of claims 12-15, wherein regression of an existing brain metastasis in the subject has been promoted.

17. The method of any one of claims 12-16, wherein the size of an existing brain metastasis in the subject has been reduced.

18. A method for treating or ameliorating a HER2 positive breast cancer in a subject in need thereof, wherein the subject has brain metastasis, the method comprising administering to the subject an effective amount of a combination therapy comprising tucatinib, capecitabine, and trastuzumab, wherein following administration of the combination therapy, the subject exhibits progression-free survival of at least 6 months following administration of the combination therapy.

19. The method of claim 18, wherein the subject exhibits progression-free survival of at least seven months following administration of the combination therapy.

20. The method of any one of claims 18 or 19, wherein the subject exhibits progression-free survival of at least nine months following administration of the combination therapy.

21. A method for treating or ameliorating a HER2 positive breast cancer in a subject in need thereof, the method comprising administering to the subject an effective amount of a combination therapy comprising tucatinib, capecitabine, and trastuzumab, wherein the subject exhibits a greater than 40% reduction in the risk of disease progression or death as compared to a subject administered trastuzumab and capecitabine alone.

22. The method of claim 21, wherein the subject administered the combination therapy comprising tucatinib, capecitabine, and trastuzumab exhibits a greater than 45% reduction in the risk of disease progression or death as compared to a subject administered trastuzumab and capecitabine alone.

23. A method for treating or ameliorating a HER2 positive breast cancer in a subject in need thereof, the method comprising administering to the subject an effective amount of a combination therapy comprising tucatinib, capecitabine, and trastuzumab, wherein the subject exhibits a greater than 30% reduction in the risk of death as compared to a subject administered trastuzumab and capecitabine alone.

24. A method for treating or ameliorating a HER2 positive breast cancer in a subject in need thereof, wherein the subject has a brain metastasis, the method comprising administering to the subject an effective amount of a combination therapy comprising tucatinib, capecitabine, and trastuzumab, wherein the subject exhibits a greater than 50% reduction in the risk of disease progression or death as compared to a subject administered trastuzumab and capecitabine alone.

25. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy for nine months, the subject has an estimated progression-free survival rate of greater than 40%.

26. The method of claim 25, wherein the subject has an estimated progression-free survival rate of greater than 45%.

27. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy for twelve months, the subject has an estimated progression-free survival rate of greater than 25%.

28. The method of claim 27, wherein the subject has an estimated progression-free survival rate of greater than 30%.

29. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy for fifteen months, the subject has an estimated progression-free survival rate of greater than 20%.

30. The method of claim 29, wherein the subject has an estimated progression-free survival rate of greater than 25%.

31. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy for twenty-four months, the subject has an estimated overall survival rate of greater than 35%.

32. The method of claim 31, wherein the subject has an estimated overall survival rate of greater than 40%.

33. A method for treating or ameliorating a HER2 positive breast cancer in a subject in need thereof, the method comprising administering to the subject an effective amount of a combination therapy comprising tucatinib, capecitabine, and trastuzumab, wherein following administration of the combination therapy for thirty months, the subject has an estimated overall survival rate of greater than 30%.

34. The method of claim 33, wherein the subject has an estimated overall survival rate of greater than 40%.

35. A method for treating or ameliorating a HER2 positive breast cancer in a subject in need thereof, wherein the subject has a brain metastasis, the method comprising administering to the subject an effective amount of a combination therapy comprising tucatinib, capecitabine, and trastuzumab, wherein following administration of the combination therapy for nine months, the subject has an estimated progression-free survival rate of greater than 30%.

36. The method of claim 35, wherein the subject has an estimated progression-free survival rate of greater than 40%.

37. A method for treating or ameliorating a HER2 positive breast cancer in a subject in need thereof, wherein the subject has a brain metastasis, the method comprising administering to the subject an effective amount of a combination therapy comprising tucatinib, capecitabine, and trastuzumab, wherein following administration of the combination therapy for twelve months, the subject has an estimated progression-free survival rate of greater than 15%.

38. The method of claim 37, wherein the subject has an estimated progression-free survival rate of greater than 20%.

39. A method for treating or ameliorating a HER2 positive breast cancer in a subject in need thereof, the method comprising administering to the subject an effective amount of a combination therapy comprising tucatinib, capecitabine, and trastuzumab and an effective amount of an anti-diarrheal agent.

40. The method of claim 39, wherein the combination therapy and the anti-diarrheal agent are administered concurrently.

41. The method of claim 39, wherein the anti-diarrheal agent is administered prior to administration of the combination therapy.

42. The method of claim 39-41, wherein the subject is exhibiting symptoms of diarrhea.

43. The method of claim 39-41, wherein the subject is not exhibiting symptoms of diarrhea.

44. A method of reducing the severity or incidents of diarrhea, or preventing diarrhea in a subject having a HER2 positive breast cancer and being treated with an effective amount of a combination therapy comprising tucatinib, capecitabine, and trastuzumab, the method comprising administering an effective amount of an anti-diarrheal agent prophylactically.

45. The method of claim 44, wherein the combination therapy and the anti-diarrheal agent are administered concurrently.

46. The method of claim 44, wherein the anti-diarrheal agent is administered prior to administration of the combination therapy.

47. A method of reducing the likelihood of a subject developing diarrhea, wherein the subject has a HER2 positive breast cancer and is being treated with an effective amount of a combination therapy comprising tucatinib, capecitabine, and trastuzumab, the method comprising administering an effective amount of an anti-diarrheal agent prophylactically.

48. The method of claim 47, wherein the combination therapy and the anti-diarrheal agent are administered concurrently.

49. The method of claim 47, wherein the anti-diarrheal agent is administered prior to administration of the combination therapy.

50. The method of any one of claims 1-49, wherein the tucatinib is administered to the subject at a dose of about 150 mg to about 650 mg.

51. The method of claim 50, wherein the tucatinib is administered to the subject at a dose of about 300 mg.

52. The method of claim 50 or 51, wherein the tucatinib is administered once or twice per day.

53. The method of claim 52, wherein the tucatinib is administered to the subject at a dose of about 300 mg twice per day.

54. The method of any one of claims 1-53, wherein the tucatinib is administered to the subject orally.

55. The method of any one of claims 1-53, wherein the capecitabine is administered to the subject at a dose of about 500 mg/m² to about 1500 mg/m².

56. The method of claim 55, wherein the capecitabine is administered to the subject at a dose of about 1000 mg/m².

57. The method of claim 55 or 56, wherein the capecitabine is administered to the subject orally.

58. The method of any one of claims 55-57, wherein the capecitabine is administered to the subject twice per day.

59. The method of any one of claims 1-58, wherein the trastuzumab is administered to the subject at a dose of about 400 mg to about 800 mg.

60. The method of claim 59, wherein the trastuzumab is administered to the subject at a dose of about 600 mg.

61. The method of claim 59 or 60, wherein the trastuzumab is administered to the subject subcutaneously.

62. The method of any one of claims 1-58, wherein the trastuzumab is administered to the subject at a dose of about 4 mg/kg to about 10 mg/kg.

63. The method of claim 62, wherein the trastuzumab is administered to the subject at a dose of about 6 mg/kg.

64. The method of claim 62, wherein the trastuzumab is administered to the subject at a dose of about 8 mg/kg.

65. The method of claim 62, wherein the trastuzumab is administered to the subject at an initial dose of about 8 mg/kg followed by subsequent doses of about 6 mg/kg.

66. The method of any one of claims 62-65, wherein the trastuzumab is administered intravenously.

67. The method of any one of claims 59-66, wherein the trastuzumab is administered once about every 1 week, once about every 2 weeks, once about every 3 weeks, or once about every 4 weeks.

68. The method of claim 67, wherein the trastuzumab is administered once about every 3 weeks.

69. The method of any one of claims 1-68, wherein the tucatinib, capecitabine and trastuzumab are administered to the subject on a 21 day treatment cycle.

70. The method of claim 69, wherein the tucatinib is administered to the subject twice per day on each day of the 21 day treatment cycle.

71. The method of claim 69 or 70, wherein the capecitabine is administered to the subject twice per day on each of days 1-14 of the 21 day treatment cycle.

72. The method of any one of claims 69-71, wherein the trastuzumab is administered to the subject once per 21 day treatment cycle.

73. The method of claim 72, wherein the dose of trastuzumab during the first 21 day treatment cycle is 8 mg/kg and the dose of trastuzumab during the subsequent 21 day treatment cycles is 6 mg/kg.

74. The method of any one of claims 1-73, wherein the subject was previously treated with at least one anticancer therapy for the breast cancer.

75. The method of claim 74, wherein the least one anticancer therapy is an anti-HER2 antibody or anti-HER2 antibody-drug conjugate.

76. The method of claim 75, wherein the at least one previous anticancer therapy is selected from the group consisting of trastuzumab, pertuzumab, ado-trastuzumab (T-DM1), and combinations thereof.

77. The method of any one of claims 74-76, wherein the subject is refractory to the previous anticancer therapy.

78. The method of claim 74-77, wherein the subject developed a brain metastasis during the previous anticancer therapy.

79. The method of any one of claims 1-78, wherein the subject has not been treated with another therapeutic agent for the breast cancer within the past 12 months.

80. The method of any one of claims 1-73, wherein the subject has not previously been treated with another therapeutic agent for the breast cancer.

81. The method of any one of claims 1-73, wherein the subject has not previously been treated with lapatinib, neratinib, afatinib, or capecitabine.
